(19) United States
(12) Patent Application Publication
(10) Pub. No.: US 2026/0337645 A1
(43) Pub. Date: Oct. 29, 2026

(54) Genetically Modified Host Cells Producing L-Serine

(71) Applicant: CysBio ApS, Hørsholm (DK)

(72) Inventors: Hemanshu Mundhada, Hørsholm (DK); Belinda Escher, Hørsholm (DK); Priyadharshini Chandrasekaran, Hørsholm (DK); Alex Toftgaard Nielsen, Hørsholm (DK); Adina-Roxana Popinceanu, Hørsholm (DK); Esther Prosper Ortega, Hørsholm (DK)

(21) Appl. No.: 19/121,625

(22) PCT Filed: Oct. 20, 2023
(86) PCT No.: PCT/EP2023/079297

§ 371 Date: Apr. 16, 2025

(30) Foreign Application Priority Data
Oct. 20, 2022 (DK) PA202270506

(51) Int. Cl. C12P 13/06; C12N 9/02; C12N 9/04; C12N 9/06; C12N 15/52

(52) U.S. Cl. CPC C12P 13/06; C12N 9/0006; C12N 9/0008; C12N 9/0016; C12N 9/0036; C12N 15/52; C12Y 101/01095; C12Y 102/01012; C12Y 104/01003

(57) ABSTRACT

The present invention relates to a genetically engineered host cell producing a metabolite from 3-phosphoglycerate through a metabolic pathway, comprising one or more genetic modifications preventing or reducing or alleviating a negative impact on production of the metabolite from intracellular accumulation of NADH and/or hydroxy glutarate (HGA), produced by one or more pathway enzymes.

### TECHNICAL FIELD

The present invention relates to the microbiological industry, and specifically to the bio-industrial production of metabolites such as L-serine and derivatives using genetically modified bacteria. More specifically the present disclosure describes recombinant host cells producing the metabolites from glyceraldehyde-3-phosphate through a metabolic pathway engineered to reduce intracellular accumulation of NADH and/or hydroxy glutarate (HGA) negative impacting on the metabolite production. Further disclosed are recombinant polynucleotides encoding recombinant polypeptides of the pathway, and cell cultures of the host cell which when cultured in fermentation methods produce the metabolites. Further disclosed are fermentation compositions the host cells and/or the metabolites thereof resulting from such methods also known as biobased compositions of metabolites.

### BACKGROUND

Genetically modified host cells producing metabolites such as L-serine are known eg. from WO2016120326 describing production of L-serine using genetically engineered microorganisms deficient in serine degradation pathways. WO2004/108894 discloses methods for producing amino acids using a genetically modified bacteria, including disclosure of a polypeptide similar to SEQ ID NO: 10 of this disclosure. WO2021/081185 describes microbial organisms having increased availability of co-factors, such as NADPH, for increasing production of various products. WO2020/0107626 describes methods of producing L-amino acids comprising culturing altered bacterial cells having increased amounts of NADPH as compared to unaltered bacterial cells whereby L-amino acids yields from said altered bacterial cells are greater than yields from unaltered bacterial cells. WO2021/195705 describes recombinant microorganisms for producing biological hydrogen and nucleic acid constructs and processes for modifying microorganisms for enabling the production of hydrogen. In this process it is suggested to replace gapA gene in E. coli with the gapC gene from Clostridium acetobutylicum. CN103436504A describing a construction method and application of Corynebacterium glutamicum strain resistant to the feedback inhibition by L-serine, by mutating a 3-phosophoglycerate dehydrogenase resistant to feedback inhibition by L-serine. The 3-phosophoglycerate dehydrogenase has a minor similarity to SEQ ID NO: 10 of this disclosure.

Further, hydroxyglutarate (HGA) is known to be produced as byproduct in many different organisms including humans. In humans, it is produced by isocitrate dehydrogenase (IDH) or phosphoglycerate dehydrogenase (SerA). These enzymes oxidize NADH to NAD+ and reduce the α-ketoglutarate (KGA) to HGA as shown in FIG. 1a.¹. The accumulation of HGA has been found in many types of cancers and has been categorized as an abnormal metabolite². In prokaryotes, the main source of HGA production is SerA. The reason for such promiscuous reactions of SerA was not known and thought to be an accidental reaction³. It was very recently shown that production of HGA is coupled to the native SerA reaction to reduce the Gibbs free energy. SerA catalyzes the first step of L-serine biosynthesis reaction wherein it oxidizes 3-phosphoglycerate (3-PG) to 3-hydroxyphosphopyruvate (3-PY). This is a highly unfavorable reaction with Gibbs free energy more than +30 KJ/mol⁴. On the other hand, the other two reactions for L-serine biosynthesis catalyzed by SerC and SerB have negative Gibbs free energy (FIG. 1b), thereby making the SerA reaction a rate determining step. To reduce the energy barrier, it was shown that oxidation of 3-PG is coupled with reduction of KGA to HGA. Using HGA oxidases such as LghO (previously YgaF) in E. coli³ and D-hydroxyglutarate dehydrogenase (D2DHH) in many Pseudomonas species⁴, HGA is oxidized back to KGA. The generated reducing equivalents are donated to cytochrome c which in turn is channelized to Electron transport chain for ATP generation or to oxygen to produce hydrogen peroxide³.

SerA has been classified into three types namely Type 1, Type 2 and Type 3 based on the presence of different protein domains¹. It is known that some SerA homologues do not have KGA reduction activity. However, such SerA proteins do not necessarily belong to one specific type or class of enzyme. For example, SerA from rat, M. tuberculosis, C. glutamicum and B. subtilis does not seem to have HGA accumulation⁴ whereas human SerA belonging to the same class has a tendency of HGA accumulation⁵. It is still not known how SerA without the promiscuous KGA reduction activity overcome the free energy barrier to drive the reaction forward efficiently.

As mentioned earlier, SerA is the first key step of L-serine biosynthesis, therefore in order to overproduce L-serine, SerA from E. coli is overexpressed in most of the published works⁶. Since the oxidation of 3-PG has been coupled with reduction of KGA (FIG. 1 B), increased L-serine production leads to higher accumulation of HGA⁷. The obvious strategy to avoid HGA accumulation is to overexpress heterologous SerA which does not have activity towards KGA as it has been shown in C. glutamicum^{4,6} or to co-express HGA oxidase to recycle HGA back to KGA as previously demonstrated.

We tested both the approaches, the expression of SerA lacking KGA reduction activity led to decline in growth and production of L-serine (example 5 and 6) while co-expression of LghO did not lead to any decline in HGA accumulation levels (data not shown). We took a unique approach wherein we hypothesized that HGA accumulation could be proportional to available NADH pool in the cell and reducing this NADH pool could in turn reduce HGA accumulation. Furthermore, this approach would lead to higher L-serine production when coupled with overexpression of SerA without or diminished KGA activity.

In literature, two strategies are applied to reduce the NADH pool: Oxidizing NADH to NAD+ using NADH oxidases (Nox)⁸ or replacing NADH generating reactions by the enzymes which would generate NADPH such as replacing NADPH dependent glutamate dehydrogenase to NADH dependent⁹ or 3-phosphoglycerate dehydrogenase to heterologous NADPH variants^{10,11}. Though all the above approaches have been tried in literature, they have never been studied with respect to their effects on HGA accumulation. We further show that reduction of the NADH pool is crucial for production of L-serine from SerA which do not have KGA reduction activity. Without the above approach, the cell growth and L-serine production are hampered. Moreover, L-serine being precursor of many amino acids such as cysteine, methionine and tryptophan and over expression of serA has been a standard strategy to enhance production of these compounds^{12,13} the above strategies would also enhance their production.

### SUMMARY

One objective of the present invention is to provide means allowing a more efficient production of metabolites from glyceraldehyde-3-phosphate, such as L-serine and derivatives thereof, microbial cells. More particularly, it is an object of the present invention to provide means allowing the production of metabolites from glyceraldehyde-3-phosphate, such as L-serine or derivatives thereof at higher nominal yield and improved mass yield. This is achieved by the finding that the production of metabolites from glyceraldehyde-3-phosphate, such as L-serine can be enhanced by engineering a microorganism to provide for prevention or reduction or alleviation of the negative impact on production of the metabolite from intracellular accumulation of NADH and/or hydroxy glutarate (HGA), produced by one or more enzymes in the pathway making the metabolite, including engineering a bacterium to have increased expression of SerA and decreased production of hydroxyglutarate.

The present disclosure describes in a first aspect genetically engineered host cell producing a metabolite from 3-phosphoglycerate through a metabolic pathway, comprising one or more genetic modifications preventing or reducing or alleviating a negative impact on production of the metabolite from intracellular accumulation of NADH and/or hydroxy glutarate (HGA), produced by one or more pathway enzymes.

In a second aspect the disclosure describes a cell culture, comprising the host cell described herein and a growth medium.

In a third aspect the disclosure describes a method for producing metabolite from 3-phosphoglycerate comprising:
a) culturing the cell culture described herein at conditions allowing the host cell to produce the metabolite from 3-phosphoglycerate; and
b) optionally recovering and/or isolating the metabolite from 3-phosphoglycerate.

In a fourth aspect the disclosure describes a fermentation composition comprising biobased metabolites of the cell culture described herein and the metabolite from 3-phosphoglycerate, wherein at least 20% by weight of the carbon is biobased.

The present invention provides in a fifth aspect a genetically engineered bacterium which has been modified to have an increased expression of a polypeptide having D-3-phosphoglycerate dehydrogenase activity (SerA) and a decreased production of hydroxyglutarate (HGA) compared to an otherwise identical bacterium that does not carry said modification.

The present invention provides in a sixth aspect a method for producing L-serine comprising: cultivating the bacterium as described in the fifths aspect in a culture medium.

### BRIEF DESCRIPTION OF THE FIGURES

FIG. 1. A. The reaction catalyzed by SerA. The native reaction includes oxidation of PGA while promiscuous reaction involves reduction of KGA. B. The potential reason for HGA production by SerA is that the overall Gibbs free energy of the reaction is reduced from 33 to 4.5 KJ/mol⁴.

FIG. 2. HGA concentrations detected in batch fermentation supernatants after 24 h incubation. The strain expressing NADH oxidase (Nox) accumulates 8 times less HGA than the strain without NADH oxidase expression.

FIG. 3. A. Schematic of NADH recycling for L-serine production via RocG.

FIG. 4A-B. L-serine concentrations detected in 48 h and 70 h fed batch fermentation supernatants of E. coli strains expressing native gapA or heterologous gapC. Both strains express either the L-serine pathway containing serACB from E. coli (A) or serCB from E. coli and serA from C. glutamicum (B).

FIG. 5. Growth curves of genetically engineered bacterium with gapC expression (serHM_608) and without gapC expression (serHM_708) obtained in 24 h batch fermentation in baffled shake flasks. The error bars are standard deviations of 2/3 biological replicates.

FIGS. 6a to 6c: Plasmid maps of all the plasmids used in the examples 1 to 5.

FIG. 7: Plasmid maps of plasmids used in Example 6.

FIG. 8: L-serine concentrations detected in batch fermentation supernatants after 24-hour incubation. The strain expressing truncated serA from rat presents increased L-serine production.

FIG. 9: L-serine concentrations detected in batch fermentation supernatants after 24-hour incubation of strains expressing different NADPH glyceraldehyde 3 phosphate dehydrogenases.

FIG. 10: Plasmid map of plasmid used in Example 8.

FIG. 11: L-serine concentrations detected in batch fermentation supernatants after 24-hour incubation. Strain expressing Nox from L. parakefiri shows increased L-serine production.

FIG. 12: Plasmids maps of all plasmids used in Example 9.

FIGS. 13a to 13d: L-serine and HGA concentrations in batch fermentation supernatants after 24-hour incubation of strains over expressing pgl and zwf in combination with serA from C. glutamicum in gapC and gapA backgrounds, and serA from E. coli in gapC and gapA backgrounds.

FIG. 14 shows effects of replacing NADH-dependent glyceraldehyde-3-phosphate dehydrogenase (gapA) with NADPH-dependent glyceraldehyde-3-phosphate dehydrogenase (gapC and gdp1) when expressing serA with no KGA reducing activity.

FIG. 15 shows the pathway for L-serine, including the steps from 3-phosohoglycerate.

FIG. 16 shows the HMP shunt pathway employing glucose-6-phosphate dehydrogenase (zwf) and 6-phosphogluconolactonase (pgl).

### DETAILED DESCRIPTION

The term “heterologous” or “recombinant” or “genetically modified” and their grammatical equivalents as used herein interchangeably about nucleotides, polypeptides and cells refers to entities “derived from a different species or cell”. For example, a heterologous or recombinant polynucleotide gene is a gene in a host cell not naturally containing that gene, i.e. the gene is from a different species or cell type than the host cell. A heterologous or recombinant polypeptide is a polypeptide produced in a host cell not naturally containing the polypeptide, i.e. the polypeptide is from a different species or cell type than the host cell. Where the terms as used herein about host cells, they refer to host cells comprising and expressing heterologous or recombinant polynucleotides. In some embodiments “recombinant” or “non-naturally occurring” when used with reference to, e.g., a host cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant host cells expressing genes that are not found within the native (non-recombinant) form of the cell or express native genes that are otherwise expressed at a different level. “Heterologous” as used herein means that a polypeptide is normally not found in or made (i.e. expressed) by the host organism, but derived from a different species.

The term “% identity” is used herein about the relatedness between two amino acid sequences or between two nucleotide sequences usings standard alignment software known in the art, and applying settings as instructed for the software, including gaps, to achieve the maximum percent identity/similarity/homology and, if necessary, considering any conservative substitutions according to the NCIUB rules (hftp://www.chem.qmul.ac.uk/iubmb/misc/naseq.html; NC-IUB, Eur. J. Biochem. (1985)) as part of the sequence identity. 5′ or 3′ extensions nor insertions (for nucleic acids) or N′ or C′ extensions nor insertions (for polypeptides) usually result in a reduction of identity, similarity or homology using such standard software. Accordingly, “Percentage of sequence identity,” “% sequence identity” and “percent identity” can be used herein to refer to comparisons between an amino acid sequence and a reference amino acid sequence. For example the “% sequence identify”, as used herein, is calculated from the two amino acid sequences as follows: The sequences are aligned using Version 9 of the Genetic Computing Group's GAP (global alignment program), using the default BLOSUM62 matrix (see below) with a gap open penalty of −12 (for the first null of a gap) and a gap extension penalty of −4 (for each additional null in the gap). After alignment, percentage identity is calculated by expressing the number of matches as a percentage of the number of amino acids in the reference amino acid sequence. The following BLOSUM62 matrix is used:

| | | | | | | | | | | | | | | | | | | | | |
|---|---|---|---|---|---|---|---|---|---|---|---|---|---|---|---|---|---|---|---|---|
| Ala | 4 | | | | | | | | | | | | | | | | | | | |
| Arg | −1 | 5 | | | | | | | | | | | | | | | | | | |
| Asn | −2 | 0 | 6 | | | | | | | | | | | | | | | | | |
| Asp | −2 | −2 | 1 | 6 | | | | | | | | | | | | | | | | |
| Cys | 0 | −3 | −3 | −3 | 9 | | | | | | | | | | | | | | | |
| Gln | −1 | 1 | 0 | 0 | −3 | 5 | | | | | | | | | | | | | | |
| Glu | −1 | 0 | 0 | 2 | −4 | 2 | 5 | | | | | | | | | | | | | |
| Gly | 0 | −2 | 0 | −1 | −3 | −2 | −2 | 6 | | | | | | | | | | | | |
| His | −2 | 0 | 1 | −1 | −3 | 0 | 0 | −2 | 8 | | | | | | | | | | | |
| Ile | −1 | −3 | −3 | −3 | −1 | −3 | −3 | −4 | −3 | 4 | | | | | | | | | | |
| Leu | −1 | −2 | −3 | −4 | −1 | −2 | −3 | −4 | −3 | 2 | 4 | | | | | | | | | |
| Lys | −1 | 2 | 0 | −1 | −3 | 1 | 1 | −2 | −1 | −3 | −2 | 5 | | | | | | | | |
| Met | −1 | −1 | −2 | −3 | −1 | 0 | −2 | −3 | −2 | 1 | 2 | −1 | 5 | | | | | | | |
| Phe | −2 | −3 | −3 | −3 | −2 | −3 | −3 | −3 | −1 | 0 | 0 | −3 | 0 | 6 | | | | | | |
| Pro | −1 | −2 | −2 | −1 | −3 | −1 | −1 | −2 | −2 | −3 | −3 | −1 | −2 | −4 | 7 | | | | | |
| Ser | 1 | −1 | 1 | 0 | −1 | 0 | 0 | 0 | −1 | −2 | −2 | 0 | −1 | −2 | −1 | 4 | | | | |
| Thr | 0 | −1 | 0 | −1 | −1 | −1 | −1 | −2 | −2 | −1 | −1 | −1 | −1 | −2 | −1 | 1 | 5 | | | |
| Trp | −3 | −3 | −4 | −4 | −2 | −2 | −3 | −2 | −2 | −3 | −2 | −3 | −1 | 1 | −4 | −3 | −2 | 11 | | |
| Tyr | −2 | −2 | −2 | −3 | −2 | −1 | −2 | −3 | 2 | −1 | −1 | −2 | −1 | 3 | −3 | −2 | −2 | 2 | 7 | |
| Val | 0 | −3 | −3 | −3 | −1 | −2 | −2 | −3 | −3 | 3 | 1 | −2 | 1 | −1 | −2 | −2 | 0 | −3 | −1 | 4 |
| | Ala | Arg | Asn | Asp | Cys | Gln | Glu | Gly | His | Ile | Leu | Lys | Met | Phe | Pro | Ser | Thr | Trp | Tyr | Val |

“Reference sequence” or “reference amino acid sequence” refers to a defined sequence to which another sequence is compared. In the context of the present invention a reference amino acid sequence may, for example, be an amino acid sequence set forth in SEQ ID NO: 5 or 6.

“Substitution” or “substituted” refers to modification of the polypeptide by replacing one amino acid residue with another, for instance the replacement of a Serine residue with a Glycine or Alanine residue in a polypeptide sequence is an amino acid substitution. When used with reference to a polynucleotide, “substitution” or “substituted” refers to modification of the polynucleotide by replacing one nucleotide with another, for instance the replacement of a cytosine with a thymine in a polynucleotide sequence is a nucleotide substitution.

“Conservative substitution”, when used with reference to a polypeptide, refers to a substitution of an amino acid residue with a different residue having a similar side chain, and thus typically involves substitution of the amino acid in the polypeptide with amino acids within the same or similar class of amino acids. By way of example and not limitation, an amino acid with an aliphatic side chain may be substituted with another aliphatic amino acid, e.g., alanine, valine, leucine, and isoleucine; an amino acid with hydroxyl side chain is substituted with another amino acid with a hydroxyl side chain, e.g., serine and threonine; an amino acid having an aromatic side chain is substituted with another amino acid having an aromatic side chain, e.g., phenylalanine, tyrosine, tryptophan, and histidine; an amino acid with a basic side chain is substituted with another amino acid with a basic side chain, e.g., lysine and arginine; an amino acid with an acidic side chain is substituted with another amino acid with an acidic side chain, e.g., aspartic acid or glutamic acid; and a hydrophobic or hydrophilic amino acid is replaced with another hydrophobic or hydrophilic amino acid, respectively.

“Non-conservative substitution”, when used with reference to a polypeptide, refers to a substitution of an amino acid in a polypeptide with an amino acid with significantly differing side chain properties. Non-conservative substitutions may use amino acids between, rather than within, the defined groups and affects (a) the structure of the peptide backbone in the area of the substitution (e.g., serine for glycine), (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

The term “pathway” or “biosynthetic pathway” or “metabolic pathway” as used herein interchangeably refers to one or more enzymes acting in concert in a live cell to convert one or more substrate precursors into a chemical product. A pathway may include one enzyme or multiple enzymes acting in sequence or in combination. A pathway including only one enzyme may also herein be referred to as “bioconversion” in particular relevant for embodiments where a host cell is fed with a precursor or substrate exogenously to be converted by the enzyme into a desired end product. Enzymes are characterized by having catalytic activity, which can change the chemical structure of the substrate(s). An enzyme may have more than one substrate and produce more than one product. The enzyme may also depend on cofactors, which can be inorganic chemical compounds or organic compounds (co-factor and/or co-enzymes) which may or may not be considered part of the pathway.

The term “in vivo”, as used herein refers to within a living cell or organism, including, for example animal, a plant, or a microorganism.

The term “in vitro” as used herein refers to outside a living cell or organism, including, without limitation, for example, in a microwell plate, a tube, a flask, a beaker, a tank, a reactor and the like.

The term “substrate” or “precursor” as used herein refers to any compound that can be converted into a different compound. For clarity, substrates and/or precursors include both compounds generated in situ by an enzymatic reaction in a cell or exogenously provided compounds, such as exogenously provided organic molecules which the host cell can metabolize into a desired compound.

The term “expression” includes any step involved in the production of a polypeptide (e.g., encoded enzyme) including, but not limited to, transcription, post-transcriptional modification, translation, post-translational modification, and secretion.

The term “expression vector” refers to a DNA molecule, either single- or double stranded, either linear or circular, which comprises a polynucleotide encoding a polypeptide and is operably linked to control sequences that provide for its expression. Expression vectors include expression cassettes for the integration of genes into a host cell as well as plasmids and/or chromosomes comprising such genes. Vectors capable of directing the expression of genes to which they are operatively linked are referred to herein as expression vectors. “Vectors” can also refer to a nucleic acid molecule capable of transporting another nucleic acid molecule to which it has been linked. One type of vector is a “plasmid”, which refers to a circular double stranded nucleic acid loop into which additional nucleic acid segments can be ligated. Certain other vectors are capable of facilitating the insertion of an exogenous nucleic acid molecule into a genome of a bacterium. Such vectors are referred to herein as “transformation vectors”. In general, vectors of utility in recombinant nucleic acid techniques are often in the form of plasmids. In the present specification, “plasmid” and “vector” can be used interchangeably as the plasmid is one of the most commonly used form of a vector. Large numbers of suitable vectors are known to those of skill in the art and commercially available.

The term “host cell” refers to any cell type that is susceptible to transformation, transfection, transduction, or the like with a nucleic acid construct or expression vector comprising a polynucleotide to be expressed in the host cell. Host cell encompasses any progeny of a parent cell including those that are not identical to the parent cell due to mutations that occur during replication.

The term “Nucleic acid” or “polynucleotide” are used interchangeably herein to denote a polymer of at least two nucleic acid monomer units or bases (e.g., adenine, cytosine, guanine, thymine) covalently linked by a phosphodiester bond, regardless of length or base modification.

The term “polynucleotide construct” refers to a polynucleotide, either single- or double stranded, which is isolated from a naturally occurring gene or is modified to contain segments of nucleic acids in a manner that would not otherwise exist in nature or which is synthetic, and which comprises a polynucleotide encoding a polypeptide and one or more control sequences.

The term “operably linked” refers to a configuration in which a control sequence is placed at an appropriate position relative to a coding polynucleotide such that the control sequence directs expression of the coding polynucleotide. More generally, “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequence. A promoter sequence is “operably-linked” to a gene when it is in sufficient proximity to the transcription start site of a gene to regulate transcription of the gene.

As used herein, “promoter” refers to a sequence of DNA, usually upstream (5′) of the coding region of a structural gene, which controls the expression of the coding region by providing recognition and binding sites for RNA polymerase and other factors which may be required for initiation of transcription. The selection of the promoter will depend upon the nucleic acid sequence of interest. A suitable “promoter” is generally one which is capable of supporting the initiation of transcription in a bacterium of the invention, causing the production of an mRNA molecule.

“Polypeptide” and “protein” are used interchangeably herein to denote a polymer of at least two amino acids covalently linked by an amide bond, regardless of length or post-translational modification (e.g., glycosylation, phosphorylation, lipidation, myristylation, ubiquitination, etc.).

Included within this definition are D- and L-amino acids, and mixtures of D- and L-amino acids.

The term “biobased” as used herein is used to characterize biobased products wherein:
a) the total carbon content of the product is at least 30%, and
b) the carbon content of a renewable raw material (biobased) is at least 20%.

As recognized by the Circular Bio-based Europe Joint Undertaking (CBE Joint Undertaking) established in 2021, developing biobased materials is essential if the EU is to reach its climate targets as set out in the European Green Deal. The present disclosure provides a methodology for efficiently providing fatty alcohols and fatty aldehydes having a high content of biobased carbon (%).

Both fossil and renewable raw materials consist mainly of carbon (C). Carbon occurs in several isotopes. Isotope ¹⁴C is radioactive and occurs naturally in all living organisms (plants, animals, etc.) in a fixed relative concentration which is nearly identical to the relative ¹⁴C concentration in the atmosphere. At this concentration, the radioactivity level of ¹⁴C is 100%. Once an organism is no longer living, this concentration, and thus the radioactivity rate, decays with a half-life of approximately 5700 years. The radioactive ¹⁴C level of an unknown substance can therefore help determine how old the carbon contained in the substance is.

“Young” carbon (0 to 10 years) derived from renewable raw materials, such as plants or animals, has a relative isotope ¹⁴C concentration which is nearly identical to the relative ¹⁴C concentration in the atmosphere and the radioactive ¹⁴C level of such young carbon is thus about 100%.

“Old” carbon (millions of years) derived from synthetic, or fossil (petrochemical) sources is greatly depleted from isotope ¹⁴C as the age of such synthetic and fossil sources far exceeds the half-life of isotope ¹⁴C which is approximately 5700 years. Hence, carbon derived from synthetic, or fossil sources has a relative isotope ¹⁴C concentration around 0% and the radioactive ¹⁴C level of such old carbon is thus about 0%.

In one embodiment the term “radioactive ¹⁴C level” refer to the total radioactive ¹⁴C level of a given substance, product, or composition, as defined above.

The isotope ¹⁴C method may be used to determine the concentration of young (renewable) materials in comparison with the concentration of old (fossil) resources. The carbon content of a renewable raw material is referred to as the “biobased carbon content”. The carbon content of a renewable raw material or the “biobased carbon content” may be determined as described below.

When measuring the biobased carbon content, the result may be reported as “% biobased carbon”. This indicates the percentage carbon from “natural” (plant or animal by-product) sources versus “synthetic” or “fossil” (petrochemical) sources. For reference, 100% biobased carbon indicates that a material is entirely sourced from plants or animal by-products and 0% biobased carbon indicates that a material did not contain any carbon from plants or animal by-products. A value in between represents a mixture of natural and fossil sources. For example: If a product has a radioactive ¹⁴C level of 80%, it means that the product consists of 80% renewable and 20% fossil carbon (C). In other words, the product is 80% biobased. The analytical measurement may be cited as “percent modern carbon (pMC)”. This is the percentage of ¹⁴C measured in the sample relative to a modern reference standard (NIST 4990C). The % Biobased Carbon content is calculated from pMC by applying a small adjustment factor for ¹⁴C in carbon dioxide in air today. It is important to note that all internationally recognized standards using ¹⁴C assume that the plant or biomass feedstocks were obtained from natural environments, pMC may be analyzed by a standard test method, such as “ASTM D6866”.

The terms “nucleotide sequence” and “polynucleotide” are used herein interchangeably.

The term “comprise” and “include” as used throughout the specification and the accompanying items as well as variations such as “comprises”, “comprising”, “includes” and “including” are to be interpreted inclusively. These words are intended to convey the possible inclusion of other elements or integers not specifically recited, where the context allows.

The articles “a” and “an” are used herein refers to one or to more than one (i.e. to one or at least one) of the grammatical object of the article. By way of example, “an element” may mean one element or more than one element.

Terms like “preferably”, “commonly”, “particularly”, and “typically” are not utilized herein to limit the scope of the itemed invention or to imply that certain features are critical, essential, or even important to the structure or function of the itemed invention. Rather, these terms are merely intended to highlight alternative or additional features that can or cannot be utilized in a particular embodiment of the present invention.

The term “cell culture” as used herein refers to a culture medium comprising a plurality of the host cells described herein. A cell culture may comprise a single strain of host cells or may comprise two or more distinct host cell strains. The culture medium may be any medium that may comprise a recombinant host, e.g., a liquid medium (i.e., a culture broth) or a semi-solid medium, and may comprise additional components, e.g., a carbon source; a nitrogen source; a phosphate source; vitamins; trace elements; salts; amino acids; nucleobases; and the like.

Term “endogenous” or “native” as used herein refers to a gene or a polypeptide in a host cell which originates from the same host cell.

The terms “substantially” or “approximately” or “about”, as used herein refers to a reasonable deviation around a value or parameter such that the value or parameter is not significantly changed. These terms of deviation from a value should be construed as including a deviation of the value where the deviation would not negate the meaning of the value deviated from. For example, in relation to a reference numerical value the terms of degree can include a range of values plus or minus 10% from that value. For example, deviation from a value can include a specified value plus or minus a certain percentage from that value, such as plus or minus 9%, 8%, 7%, 6%, 5%, 4%, 3%, 2%, or 1% from the specified value.

Where a numerical limit or range is stated herein, the endpoints are included. Also, all values and sub ranges within a numerical limit or range are specifically included as if explicitly written out.

The term “and/or” as used herein is intended to represent an inclusive “or”. The wording X and/or Y is meant to mean both X or Y and X and Y. Further the wording X, Y and/or Z is intended to mean X, Y and Z alone or any combination of X, Y, and Z.

The term “isolated” as used herein about a compound, refers to any compound, which by means of human intervention, has been put in a form or environment that differs from the form or environment in which it is found in nature. Isolated compounds include but are not limited to compounds of the disclosure for which the ratio of the compounds relative to other constituents with which they are associated in nature is increased or decreased. In an important embodiment the amount of compound is increased relative to other constituents with which the compound is associated in nature. In an embodiment the compound of the disclosure may be isolated into a pure or substantially pure form. In this context a substantially pure compound means that the compound is separated from other extraneous or unwanted material present from the onset of producing the compound or generated in the manufacturing process. Such a substantially pure compound preparation contains less than 10%, such as less than 8%, such as less than 6%, such as less than 5%, such as less than 4%, such as less than 3%, such as less than 2%, such as less than 1%, such as less than 0.5% by weight of other extraneous or unwanted material usually associated with the compound when expressed natively or recombinantly. In an embodiment the isolated compound is at least 90% pure, such as at least 91% pure, such as at least 92% pure, such as at least 93% pure, such as at least 94% pure, such as at least 95% pure, such as at least 96% pure, such as at least 97% pure, such as at least 98% pure, such as at least 99% pure, such as at least 99.5% pure, such as 100% pure by weight.

The term “cDNA” refers to a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

The term “GAPDH” as used herein refers to a glyceraldehyde-3-phosphate dehydrogenase enzyme converting glyceraldehyde 3-phosphate into 1,3-bisphosphoglycerate; under the co-conversion of NAD⁺ or NADP⁺ into NADH or NADPH respectively. GapA is an example of a GAPDH producing 1,3-bisphosphoglycerate under co-conversion of NAD⁺ into NADH, while GapC is an example of a GAPDH producing 1,3-bisphosphoglycerate under co-conversion of NADP⁺ into NADPH. The term “Nox” as used herein refers to an NADH oxidase enzyme converting NADH to NAD⁺.

The term “PGDH” or “3-PGDH” or “PHGDH” as used herein refers to a 3-phosphoglycerate dehydrogenase enzyme that catalyzes the conversion of 3-phosphoglycerate into 3-phosphohydroxypyruvate, under the simultaneous reduction of NAD+ to NADH. An example of PGDH is SerA in the serine pathway.

The term “GDH” as used herein refers to a glutamate dehydrogenase enzyme that catalyzes the conversion of α-ketoglutarate into glutamate.

The term “deletion” as used herein in the context of polynucleotides and genes refers to the manipulation of a gene so that it is no longer expressed in a host cell. “Deletion” or “deleted”, when used with reference to a polypeptide, refers to modification of the polypeptide by removal of one or more amino acids in the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the polypeptide while retaining enzymatic activity and/or retaining the improved properties of an engineered enzyme. Deletions can be directed to the internal portions and/or terminal portions of the polypeptide, in various embodiments, the deletion can comprise a continuous segment or can be discontinuous.

The term “disruption” as used herein refers to manipulation of a gene or any of the machinery participating in the expression the gene, so that it is no longer expressed in a host cell.

The term “attenuation” as used herein refers to manipulation of a gene or any of the machinery participating in the expression the gene, so that it the expression of the gene is reduced as compared to expression without the manipulation.

“Insertion” or “inserted”, when used with reference to a polypeptide, refers to modification of the polypeptide by addition of one or more amino acids to the reference polypeptide. Insertions can comprise addition of 1 or more amino acids, 2 or more amino acids, 5 or more amino acids, 10 or more amino acids, 15 or more amino acids, or 20 or more amino acids. Insertions can be in the internal portions of the polypeptide, or to the carboxy or amino terminus. The insertion can be a contiguous segment of amino acids or separated by one or more of the amino acids in the reference polypeptide.

The phrase “bacterium which has been modified to have an increased expression of a polypeptide having D-3-phosphoglycerate dehydrogenase activity (serA) and a decreased production of HGA” as used herein means that the bacterium has been modified in such a way that a) the modified bacterium has a higher level of expression of a D-3-phosphoglycerate dehydrogenase (serA) as compared to an otherwise identical bacterium that does not carry said modification, and b) the modified bacterium has a decreased production of HGA as compared to an otherwise identical bacterium that does not carry said modification. It is to be understood that “the modification” may encompass one or more separate genetic modifications to achieve the stated effects.

The presence or absence of a gene on the chromosome of a bacterium can be detected by well-known methods, including PCR, Southern blotting, and the like. In addition, the level of gene expression can be estimated by measuring the amount of mRNA transcribed from the gene using various well-known methods, including Northern blotting, quantitative RT-PCR, and the like. The amount of the protein encoded by the gene can be measured by well-known methods, including SDS-PAGE followed by an immunoblotting assay (Western blotting analysis), and the like.

“Genetically engineered bacterium” as used herein means a bacterium in which a genetic modification has been introduced, such as introduction of a new gene, additional copies of a gene, a modified gene, changes in the sequences regulating the expression of the gene, and the like. As used herein, “L-serine derivative” refers to a compound, such as an amino acid, resulting from reaction of L-serine at the amino group or the carboxy group or hydroxyl group, or from the replacement of any hydrogen of L-serine by a heteroatom. Non-limiting examples of a “L-serine derivative” include L-cysteine, L-methionine, L-glycine, O-acetylserine, L-tryptophan, thiamine, ethanolamine and ethylene glycol. Further examples of a “L-serine derivative” are described by Chemical Entities of Biological Interest (ChEBI) [https://www.ebi.ac.uk/chebi/init.do], for example, under ChEBI ID CHEBI: 84135.

All methods described herein can be performed in any suitable order of steps unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

All percentages, ratios and proportions herein are by weight, unless otherwise specified. A weight percent (weight %, also as wt. %) of a component, unless specifically stated to the contrary, is based on the total weight of the composition in which the component is included (e.g., on the total amount of the reaction mixture).

Unless specifically defined herein, all technical and scientific terms used have the same meaning as commonly understood by a skilled artisan in the fields of biochemistry, genetics, and microbiology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, and recombinant DNA, which are available to the person skilled in the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

### Genetically Engineered Host Cell

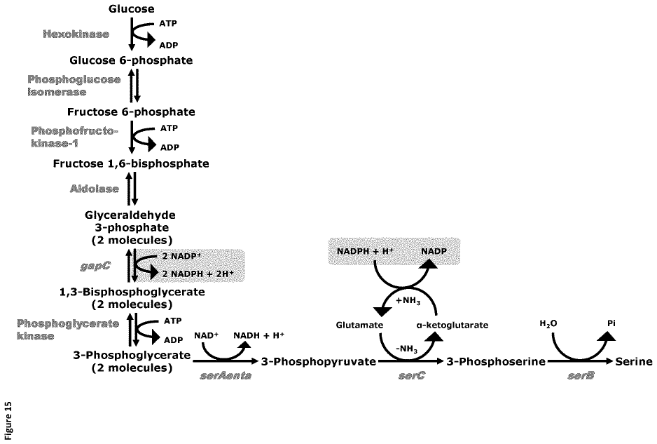

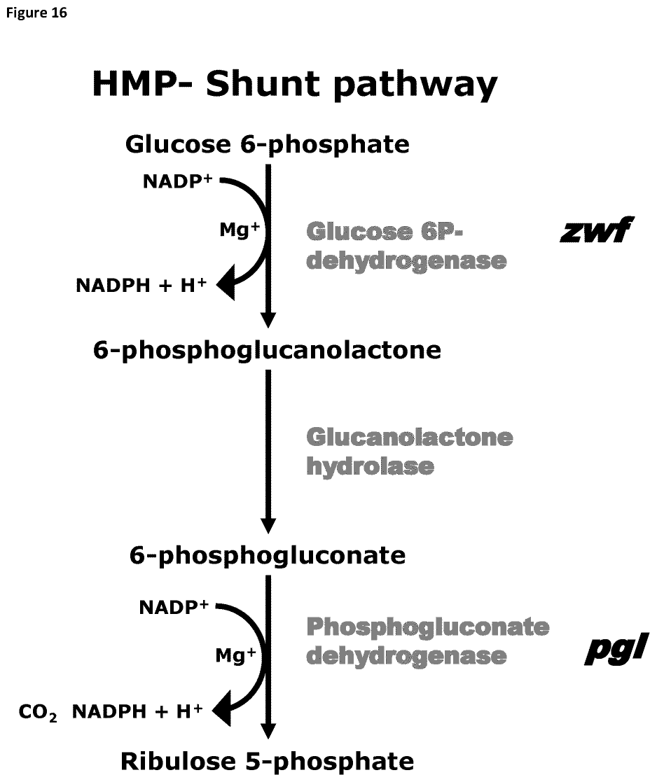

As described in the first aspect, supra, the genetically engineered host cell described herein produces a metabolite from 3-phosphoglycerate through a metabolic pathway, comprising one or more genetic modifications preventing or reducing or alleviating a negative impact on production of the metabolite from intracellular accumulation of NADH and/or hydroxy glutarate (HGA), produced by one or more pathway enzymes. The metabolite is in a preferred embodiment L-serine or a derivative thereof. The pathway for L-serine is shown in FIG. 15.

In some embodiments the host cell is genetically engineered to comprise one or more, optionally two or more, optionally three or more, optionally four or more, optionally five or more, optionally seven genetic modifications selected from:
I. expression of a first heterologous NADPH generating enzyme converting glyceraldehyde 3-phosphate into 1,3-bisphosphoglycerate or a downstream precursor in the metabolite pathway;
II. expression of a heterologous enzyme converting NADH to NAD+;
III. expression of a heterologous enzyme in the metabolite pathway having a reduced or eliminated NADH consuming side activity compared to a corresponding pathway enzyme native to the host cell;
IV. expression of a heterologous enzyme converting a side product of an enzyme in the metabolite pathway into a substrate of a metabolite pathway enzyme consuming NADH or NADPH;
V. overexpression of a native enzyme converting a side product of an enzyme in the metabolite pathway into a substrate of a metabolite pathway enzyme consuming NADH or NADPH;
VI. expression of a second heterologous NADPH producing enzyme, which is not comprised in the metabolite pathway; and/or
VII. overexpression of a native NADPH producing enzyme, which is not comprised in the metabolite pathway.

The present inventors have found that inhibiting accumulation of NADH in the cell improves the downstream efficiently of pathways producing metabolites from 3-phosphoglycerate, in particular L-serine and/or derivatives thereof, so in a further embodiment the genetic modification of the host cell comprises expression of the first heterologous NADPH generating enzyme converting glyceraldehyde 3-phosphate into 1,3-bisphosphoglycerate or a downstream precursor in the metabolite pathway.

The present inventors have also found that it is particularly advantageous and synergistic for the host cell production of metabolites from 3-phosphoglycerate, to combine two or more of the modifications I) to VII), supra, and in a further embodiment the genetic modification of the host cell comprises:
a) expression of the first heterologous NADPH generating enzyme converting glyceraldehyde 3-phosphate into 1,3-bisphosphoglycerate or a downstream precursor in the metabolite pathway; and
b) expression of the heterologous metabolite pathway enzyme having a reduced or eliminated NADH consuming side activity compared to a corresponding metabolite pathway enzyme native to the host cell.

The present inventors have also found that it is particularly advantageous and synergistic for the host cell production of metabolites from 3-phosphoglycerate, to combine three or more of the modifications I) to VII), supra, and in a further embodiment the genetic modification of the host cell comprises:
a) expression of the first heterologous NADPH generating enzyme converting glyceraldehyde 3-phosphate into 1,3-bisphosphoglycerate or a downstream precursor in the metabolite pathway;
b) expression of the heterologous metabolite pathway enzyme having a reduced or eliminated NADH consuming side activity compared to a corresponding metabolite pathway enzyme native to the host cell; and
c) expression of the heterologous enzyme converting NADH to NAD+.

The present inventors have also found that it is particularly advantageous and synergistic for the host cell production of metabolites from 3-phosphoglycerate, to combine four or more of the modifications I) to VII), supra, and in a further embodiment the genetic modification of the host cell comprises:
a) expression of the first heterologous NADPH generating enzyme converting glyceraldehyde 3-phosphate into 1,3-bisphosphoglycerate or a downstream precursor in the metabolite pathway;
b) expression of the heterologous metabolite pathway enzyme having a reduced or eliminated NADH consuming side activity compared to a corresponding metabolite pathway enzyme native to the host cell;
c) expression of the heterologous enzyme converting NADH to NAD+; and
d) expression of the heterologous enzyme and/or overexpression of the native enzyme converting the side product of the enzyme in the metabolite pathway into the substrate of the metabolite pathway enzyme consuming NADH or NADPH.

The present inventors have also found that it is particularly advantageous and synergistic for the host cell production of metabolites from 3-phosphoglycerate, to combine five more of the modifications I) to VII), supra, and in a further embodiment the genetic modification of the host cell comprises:
a) expression of the first heterologous NADPH generating enzyme converting glyceraldehyde 3-phosphate into 1,3-bisphosphoglycerate or a downstream precursor in the metabolite pathway;
b) expression of the heterologous metabolite pathway enzyme having a reduced or eliminated NADH consuming side activity compared to a corresponding metabolite pathway enzyme native to the host cell;
c) expression of the heterologous enzyme converting NADH to NAD+;
d) expression of the heterologous enzyme and/or overexpression of the native enzyme converting the side product of the enzyme in the metabolite pathway into a substrate of the metabolite pathway enzyme consuming NADH or NADPH; and
e) expression of the second heterologous NADPH producing enzyme and/or overexpression of the native NADPH producing enzyme, any of which are not comprised in the metabolite pathway.

In a further embodiment the first heterologous NADPH generating enzyme, the heterologous enzyme having a reduced or eliminated NADH consuming side activity, the heterologous enzyme converting the side product into a pathway substrate and/or the second heterologous enzyme producing NADPH partially or completely replaces enzymes native to the host cell. Such native enzymes can be part of metabolite pathway or not.

In some embodiments, the NADPH pool of the genetically engineered bacterium is increased by the recombinant expression of a NADPH dependent polypeptide having Glyceraldehyde-3-phosphate dehydrogenase activity with or without additional ATP generation.

In a further embodiment the first heterologous NADPH generating enzyme converting glyceraldehyde 3-phosphate into 1,3-bisphosphoglycerate or a downstream precursor in the metabolite pathway can be a bisphoshoglycerate synthase or a glyceraldehyde-3-phosphate dehydrogenase (GAPDH), both converting glyceraldehyde 3-phosphate into 1,3-bisphosphoglycerate; under the co-conversion of NADP+ into NADPH. SEQ ID NOs: 23 to 46 disclose some exemplary glyceraldehyde-3-phosphate dehydrogenases, and in some embodiments the GAPDH enzyme comprises a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the GAP comprised in any one of SEQ ID NO: 23 to 46. Particularly the GAPDH of SEQ ID NO: 38 to 46 are useful. In particular the GAPDH is GapC or a NADP dependent variant thereof that comprises a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the GAPDH comprised in SEQ ID NO: 43.

In some embodiments, the NADH pool of the genetically engineered bacterium is reduced by the recombinant expression of a heterologous NADH oxidase, so in a further embodiment the heterologous enzyme converting NADH to NAD+ is a NADH oxidase (Nox). SEQ ID NOs: 49 to 56 disclose some exemplary NADH oxidases and in further embodiments the Nox comprises a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the Nox comprised in any one of SEQ ID NO: 49 to 56.

In further embodiments the metabolite pathway enzyme having a reduced or eliminated NADH consuming side activity is a 3-phosphoglycerate dehydrogenase (PGDH), also known as a D-3-phosphoglycerate dehydrogenase and the side activity is conversion of α-ketoglutarate (α-KGA) into α-hydroxyglutarate (α-HGA). D-3-Phosphoglycerate dehydrogenase (PGDH) converts D-3-phosphoglycerate (PGA) to phosphohydroxypyruvate (PHP) in the first step of L-serine biosynthesis. This reaction is reversible, and some PGDHs can use α-ketoglutarate (αKG) instead of PHP in the reverse direction to produce α-hydroxyglutarate.

Preferably the PGDH is heterologous and more preferably the heterologous PGDH is overexpressed compared to a native PGDH having a side activity of converting α-ketoglutarate (α-KGA) into α-hydroxyglutarate. The overexpression of the heterologous PGDH may be 10% to 10.000% compared to the native PGDH such as 50% to 5000%, such as 100% to 1000%. PGDH or D-3-phosphoglycerate dehydrogenase (SerA) has been classified into three types, namely Type 1, Type 2 and Type 3, based on the presence of different protein domains 1. Some D-3-phosphoglycerate dehydrogenases do not have the KGA reduction activity. However, such D-3-phosphoglycerate dehydrogenase do not necessarily belong to one type or class of enzyme. For example, SerA from Rattus norvegicus, M. tuberculosis, C. glutamicum and B. subtilis does not seem to have HGA accumulation 4, whereas human SerA has tendency of HGA accumulation although it belongs to the same class 14. Three enzymes have been found to be able to utilize αKG as a substrate: PDGH from E. coli, Pseudomonas stutzeri, and Saccharomyces cerevisiae, which are all type II PGDH's. It is hypothesized that PGDH's are not able to use αKG as a substrate are type I and type III PGDH's. The present inventors have found that for each of the three types of PGDH several regions facing the active site contains conserved motifs in both type I and II PHDG's. One such motif is the G/ARAGV and GCFCI motif in type I and II PGDHs respectively. One distinct, and highly conserved, difference between these two motives is the residue type at the second position. In type I PGDHs this is an arginine residue whereas it is most often a cysteine residue in Type II enzymes. For M. tuberculosis and E. coli PGDH structures with PHP and αKG bound, respectively, it has been found that both the arginine and cysteine residues are facing the active site, indicating that these residues are involved in controlling substrate specificity, and hence the ability to use αKG as a substrate. Further it has been found that using site-directed mutagenesis to substitute the arginyl side chain in M. tuberculosis PGDH with other selected amino acid side chains such as alanine and leucine the removal of the cationic group of Arg 72 in M. tuberculosis PGDH change the specificity from not accepting αKG as a substrate to accepting αKG as a substrate. However, replacement of the arginyl side chain with another cationic moiety (lysyl side chain) do not result in a changed enzyme specificity. These results clearly show that it is the presence of a cationic side chain at the second position in the conserved motif which renders the enzyme unable to use αKG. Accordingly, in a preferred embodiment, the heterologous PGDH comprises a conserved region facing the active site which comprises the motif G/AXAGV, wherein the underlined residue X is a cationic residue, preferably at host cell intracellular pH, positioned corresponding to position 129 of the PGDH from Mycobacterium tuberculosis (SEQ ID NO: 10). Useful PDGH's are those particularly where the underlined X is selected from Arginine, Leucine or Histidine, more particularly Arginine. Additionally or alternatively useful heterologous PGDH's are those which comprise a conserved region facing the active site, which does not comprises the motif GCFCI, wherein the underlined cysteine is positioned corresponding to position 129 of the PGDH from Mycobacterium tuberculosis (SEQ ID NO: 10).

In a further embodiment the heterologous PGDH is mutant PGDH, optionally native to the host cell, modified to reduce or eliminate the NADH consuming side activity compared to the unmodified PGDH, for example by replacing a cysteine positioned corresponding to position 129 of the PGDH from Mycobacterium tuberculosis (SEQ ID NO: 10), optionally in a motif GCFCI, with a cationic residue, such as Arg, Leu or His, particularly Arg.

In some embodiments, the heterologous PGDH can be a type I or type III PGDH, optionally a microbial type I or a type III PGDH and moreover it may be a SerA enzyme.

SEQ ID NOs: 1-22 disclose some exemplary D-3-phosphoglycerate dehydrogenases, and particularly useful PGDH enzymes are those which comprise a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the PGDH comprised in any one of SEQ ID NO: 1 to 22. More particularly the PGDH is not producing HGA or is insensitive to L-serine feedback and comprises a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the PGDH comprised in any one of SEQ ID NO: 6 to 22. In further embodiments the PGDH is not producing HGA and comprises a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the PGDH comprised in any one of SEQ ID NO: 6 to 15. In still further embodiments the PGDH is a non-HGA producing C. glutamicum PGDH or a derivative thereof comprising a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the PGDH comprised in SEQ ID NO: 14, or 19 to 21 in particular SEQ ID NO: 14.

In a further embodiment comprises both a heterologous PGDH as described, supra, in particular serA from C. glutamicum (SEQ ID NO: 14, or 19 to 21 and a heterologous GAPDH as described supra, in particular GapC (SEQ ID NO: 43. As indicated above, the present invention is inter alia based on the finding that the production of L-serine can be enhanced by e.g. increasing expression of SerA and decreasing the production of hydroxyglutarate (HGA).

The decreased production of HGA may be realized by a modification increasing the cytosolic NADPH pool and/or reducing the cytosolic NADH pool compared to an otherwise identical bacterium that does not carry said modification.

The decreased production of HGA may also be realized by a modification causing expression of a polypeptide having D-3-phosphoglycerate dehydrogenase activity with reduced or no HGA production.

Accordingly, the present invention provides a genetically engineered bacterium, especially a bacterium having an ability to produce L-serine, wherein said bacterium has been modified to have increased expression of a polypeptide having D-3-phosphoglycerate dehydrogenase activity (SerA) and a decreased production of HGA compared to an otherwise identical bacterium that does not carry said modification.

In the embodiment where the enzyme converting a side product of an enzyme in the metabolite pathway into the substrate of a metabolite pathway enzyme, the said metabolite pathway enzyme is preferably a glutamate dehydrogenase (GDH), the side product is preferably α-ketoglutarate, and the substrate is glutamate. In a more specific embodiment the GDH comprises a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the GDH comprised in any one of SEQ ID NO: 57 to 66.

In the embodiment where the host cell expresses a second heterologous NADPH producing enzyme and/or overexpresses a native NADPH producing enzyme, any of which are not comprised in the metabolite pathway, the second heterologous or the native NADPH producing enzyme is preferably a Glucose-6-phosphate dehydrogenase and/or a 6-phosphogluconolactonase, respectively. In a more specific embodiment the glucose-6-phosphate dehydrogenase comprises a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the Glucose-6-phosphate dehydrogenase comprised in SEQ ID NO: 47. This glucose-6-phosphate dehydrogenase is also known as “zwf”. In a more specific embodiment the 6-phosphogluconolactonase comprises a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the 6-phosphogluconolactonase comprised in SEQ ID NO: 48. This 6-phosphogluconolactonase is also known as “pgl”.

In a further embodiment the host cell expresses one or more heterologous or native metabolite pathway enzymes selected from:
a) phosphoserine aminotransferase (PSAT) converting 3-phosphohydroxypyruvate into phosphoserine; and
b) Phosphoserine phosphatase (PSPH) converting phosphoserine into L-serine.

The PSAT can be a SerC enzyme and in some embodiments comprise a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the SerC comprised in SEQ ID NO: 117.

The PSPH can be a SerB and in some embodiments comprise a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the SerB comprised in SEQ ID NO: 118.

The heterologous enzymes expressed by the host cell as described herein can be (i) enzymes from a species different from the host cell, (ii) a mutated enzymes from a species different from the host cell, and/or (iii) a mutated enzyme native to the host cell. In further embodiments the host cell further comprises at least one transporter molecule facilitating transport of the metabolite or any of its precursors. In further embodiments, one or more native or endogenous genes of the hos cell is attenuated, disrupted and/or deleted, such as a native gene encoding a NADH dependent GAPDH or a α-HGA producing PHDH. In further embodiments, the host cell further comprises at least 2 copies of one or more polynucleotides encoding one or more metabolite pathway enzymes. In further embodiments, the host cell is further genetically modified to provide an increased amount of a substrate for one or more metabolite pathway enzymes. In further embodiments the host cell is further genetically modified to exhibit increased tolerance towards one or more precursors, substrates, intermediates, or product molecules from the metabolite pathway.

In further embodiments the host cell of any preceding claim wherein the host cell is a prokaryotic cells, optionally a bacterium. The prokaryotic cell can be a Pseudomonadota, optionally of the class gammaproteobacteria, optionally of the family Enterobacteriaceae, optionally of the genus Escherichia, optionally of the species Escherichia coli. In some embodiments the genetically engineered bacterium belongs to the Enterobacteriaceae family. In some embodiments the genetically engineered bacterium belongs to the genus Escherichia. In some embodiments the genetically engineered bacterium is Escherichia coli. Additionally or alternatively the prokaryotic cell is an Actinomycetota, optionally of the class Actinobacteria, optionally of the family Corynebacteriaceae, optionally of the genus Corynebacterium, optionally of the species Corynebacterium glutamicum. In some embodiments the genetically engineered bacterium is Corynebacterium glutamicum.

In an embodiment, the genetically engineered bacterium has been modified for reduced production of hydroxyglutarate by increasing the cytosolic NADPH pool and/or reducing the cytosolic NADH pool compared to an otherwise identical bacterium that does not carry said modification. In another embodiment, the genetically engineered bacterium has been modified for reduced production of hydroxyglutarate by expression of a polypeptide having D-3-phosphoglycerate dehydrogenase activity with reduced or no activity of hydroxyglutarate production.

The present disclosure describes in separate aspects a cell culture, comprising the host cell described herein and a growth medium and methods for producing the metabolite from 3-phosphoglycerate by culturing the cell culture at conditions allowing the host cell to produce the metabolite; and optionally recovering and/or isolating the metabolite. Suitable growth mediums for prokaryotic cell are well known in the art. The cell culture can be cultivated in a nutrient medium using methods known in the art at conditions suitable for production of the metabolite and/or its precursors and/or for propagating cell count. For example, the culture may be cultivated by shake flask cultivation, or small-scale or large-scale fermentation (including continuous, batch, fed-batch, or solid-state fermentations) in laboratory or industrial fermenters in a suitable medium and under conditions allowing the host cells to grow and/or propagate, optionally to be recovered and/or isolated.

The cultivation can take place in a suitable nutrient medium comprising carbon and nitrogen sources and inorganic salts, using procedures known in the art. Suitable media are available from commercial suppliers or may be prepared according to published recipes (e.g. from catalogues of the American Type Culture Collection). The selection of the appropriate medium may be based on the choice of host cell and/or based on the regulatory requirements for the host cell. Such media are available in the art. The medium may, if desired, contain additional components favouring the host cells over other potentially contaminating microorganisms. Accordingly, in an embodiment a suitable nutrient medium comprises a carbon source (e.g. glucose, maltose, molasses, starch, cellulose, xylan, pectin, lignocellolytic biomass hydrolysate, etc.), a nitrogen source (e. g. ammonium sulphate, ammonium nitrate, ammonium chloride, etc.), an organic nitrogen source (e.g. yeast extract, malt extract, peptone, etc.) and inorganic nutrient sources (e.g. phosphate, magnesium, potassium, zinc, iron, etc.). Culturing of the host cell may be performed over a period of about 0.5 to about 30 days. The cultivation process may be a batch process, continuous or fed-batch process, suitably performed at a temperature in the range of 0-100 °C or 10-80 °C, for example, from about 20 °C to about 50 °C and/or at a pH, for example, from about 2 to about 10. Preferred fermentation conditions for prokaryotic host cells are a temperature in the range of from about 25 °C to about 55 °C and at a pH of from about 3 to about 9. The appropriate conditions are usually selected based on the choice of host cell. Accordingly, in an embodiment the method of the disclosure further comprises one or more elements selected from:
a) culturing the cell culture in a nutrient medium;
b) culturing the cell culture under aerobic or anaerobic conditions
c) culturing the cell culture under agitation;
d) culturing the cell culture at a temperature of between 25 to 50 °C;
e) culturing the cell culture at a pH of between 3-9; and
f) culturing the cell culture for between 10 hours to 30 days.

The cell culture of the disclosure may be recovered and or isolated using methods known in the art. For example, the metabolite may be recovered from the nutrient medium by conventional procedures including, but not limited to, centrifugation, filtration, spray-drying, or lyophilization. In a particular embodiment the method includes a recovery and/or isolation step comprising separating a liquid phase of the cell or cell culture from a solid phase of the cell or cell culture to obtain a supernatant comprising the metabolite and/or subjecting the supernatant to one or more steps selected from:
a) disrupting the cells of the cell culture to release intracellular metabolite into the supernatant;
b) separating the supernatant from the solid phase of the cell culture, such as by filtration or gravity separation;
c) contacting the supernatant with one or more adsorbent resins to obtain at least a portion of the produced metabolite;
d) contacting the supernatant with one or more ion exchange or reversed-phase chromatography columns in order to obtain at least a portion of the metabolite;
e) extracting the metabolite; and/or
f) precipitating the metabolite by crystallization or evaporating the solvent of the liquid phase; and optionally isolating the metabolite by filtration or gravity separation;
   thereby recovering and/or isolating the metabolite.

In one embodiment the method further comprises feeding the cell culture with one or more precursor or substrates in the pathway of the metabolite.

In another embodiment the method comprises one or more in vitro steps in the process of producing the metabolite. In particular where the metabolite is not the desired end products further steps may be added to the method described herein either chemically or biologically/enzymatically modifying the metabolite

The method can further comprise recovering the metabolite and mixing it with one or more carriers, agents, additives, adjuvants and/or excipients to produce a biopesticide composition.

### Method of the Invention

In a special embodiment the present invention also provides methods for production of L-serine or a L-serine derivatives using a genetically engineered bacterium according to the present invention. Particularly, the present invention provides a method for production of L-serine or a L-serine derivatives which comprises cultivating a genetically engineered bacterium as detailed herein in a culture medium. According to certain embodiments, the present invention provides a method for producing L-serine. Particularly, the present invention provides a method for producing L-serine, said method comprises cultivating a genetically engineered bacterium as detailed herein in a culture medium. The method may further comprise isolating L-serine from the culture medium. According to certain embodiments, the present invention provides a method for producing a L-serine derivative. Particularly, the present invention provides a method for producing a L-serine derivative, said method comprises cultivating a genetically engineered bacterium as detailed herein in a culture medium. The L-serine derivative may be selected from the group consisting of L-cysteine, L-methionine, L-glycine, O-acetylserine, L-tryptophan, thiamine, ethanolamine and ethylene glycol. The method may further comprise isolating the L-serine derivative from the culture medium. According to certain embodiments, the present invention provides a method for producing L-cysteine. Particularly, the present invention provides a method for producing L-cysteine, said method comprises cultivating a genetically engineered bacterium as detailed herein in a culture medium. The method may further comprise isolating L-cysteine from the culture medium. According to certain embodiments, the present invention provides a method for producing L-methionine. Particularly, the present invention provides a method for producing L-methionine; said method comprises cultivating a genetically engineered bacterium as detailed herein in a culture medium. The method may further comprise isolating L-methionine from the culture medium. According to certain embodiments, present invention provides a method for producing L-glycine. Particularly, the present invention provides a method for producing L-glycine; said method comprises cultivating a genetically engineered bacterium as detailed herein in a culture medium. The method may further comprise isolating L-glycine from the culture medium. According to certain embodiments, present invention provides a method for producing O-acetylserine. Particularly, the present invention provides a method for producing O-acetylserine, said method comprises cultivating a genetically engineered bacterium as detailed herein in a culture medium. The method may further comprise isolating O-acetylserine from the culture medium. According to certain embodiments, present invention provides a method for producing L-tryptophan. Particularly, the present invention provides a method for producing L-tryptophan; said method comprises cultivating a genetically engineered bacterium as detailed herein in a culture medium. The method may further comprise isolating L-tryptophan from the culture medium.

According to certain embodiments, present invention provides a method for producing L-thiamine. Particularly, the present invention provides a method for producing L-thiamine, said method comprises cultivating a genetically engineered bacterium as detailed herein in a culture medium. The method may further comprise isolating thiamine from the culture medium. According to certain embodiments, present invention provides a method for producing ethanolamine. Particularly, the present invention provides a method for producing ethanolamine; said method comprises cultivating a genetically engineered bacterium as detailed herein in a culture medium. The method may further comprise isolating ethanolamine from the culture medium. According to certain embodiments, present invention provides a method for producing ethylene glycol. Particularly, the present invention provides a method for producing ethylene glycol; said method comprises cultivating a genetically engineered bacterium as detailed herein in a culture medium. The method may further comprise isolating ethylene glycol from the culture medium. The culture medium employed may be any conventional medium suitable for culturing a bacterium cell in question, and may be composed according to the principles established in the art. The medium will usually contain all nutrients necessary for the growth and survival of the respective bacterium, such as carbon and nitrogen sources and other inorganic salts. Suitable media, e.g. minimal or complex media, are available from commercial suppliers, or may be prepared according to published receipts, e.g. the American Type Culture Collection (ATCC) Catalogue of strains. Non-limiting standard medium well known to the skilled person include Luria Bertani (LB) broth, Sabouraud Dextrose (SD) broth, MS broth, Yeast Peptone Dextrose, BMMY, GMMY, or Yeast Malt Extract (YM) broth, which are all commercially available. A non-limiting example of suitable media for culturing bacterial cells, such as E. coli cells, including minimal media and rich media such as Luria Broth (LB), M9 media, M17 media, SA media, MOPS media, Terrific Broth, YT and others. The carbon source may be any suitable carbon substrate known in the art, and in particularly any carbon substrate commonly used in the cultivation of bacteria and/or fermentation. Non-limiting examples of suitable fermentable carbon substrates are C5 sugars (such as arabinose or xylose), C6 sugars (such as glucose), acetate, glycerol, plant oils, sucrose, yeast extract, peptone, casamino acids or mixtures thereof. A carbon source of particular interest is a C6 sugar such as glucose. As the nitrogen source, various ammonium salts such as ammonia and ammonium sulfate, other nitrogen compounds such as amines, a natural nitrogen source such as peptone, soybean-hydrolysate, and digested fermentative microorganism can be used. As minerals, potassium monophosphate, magnesium sulfate, sodium chloride, ferrous sulfate, manganese sulfate, calcium chloride, and the like can be used. The cultivation can be preferably performed under aerobic conditions, such as by a shaking culture, and by a stirring culture with aeration, at a temperature of about 20 to about 40 °C, such as about 30 to 38 °C, preferably about 37 °C. The pH of the culture is usually from about 5 and about 9, such as from about 6.5 and 7.5. The pH of the culture can be adjusted with ammonia, calcium carbonate, various acids, various bases, and buffers. Usually, 1 to 5-day cultivation leads to accumulation of L-serine in the culture medium. After cultivation, solids such as cells can be removed from the culture medium by centrifugation or membrane filtration. L-serine or the L-serine derivative can be collected by conventional method for isolation and purification chemical compounds from a medium. Well-known purification procedures include, but are not limited to, centrifugation or filtration, precipitation, ion exchange, chromatographic methods such as e.g. ion exchange chromatography or gel filtration chromatography, and crystallization methods. The present invention thus provides L-serine or a L-serine derivative obtainable by a method as detailed herein.

In a further aspect a fermentation composition is provided for, which comprises the metabolites of the cell culture described herein and the metabolite from 3-phosphoglycerate, wherein at least 20% by weight of the carbon is biobased. In some embodiments, the composition comprises at least 50% biobased carbon, such as at least 55%, such as at least 60%, such as at least 65%, such as at least 70%, such as at least 75%, such as at least 80%, such as at least 85%, such as at least 90%, such as at least 95%, such as at least 99%, such as at least 100%. In other embodiments, the composition comprises at least 20% biobased carbon, such as at least 30% biobased carbon, such as at least 40% biobased carbon, such as at least 50% biobased carbon, such as at least 60% biobased carbon, such as at least 70% biobased carbon, such as at least 75% biobased carbon, such as at least 80% biobased carbon, such as at least 85% biobased carbon, such as at least 90% biobased carbon, such as at least 95% biobased carbon, such as 100% biobased carbon. In still other embodiments the composition comprises from 20% to 100% biobased carbon, such as from 30% to 100% biobased carbon, such as from 40% to 100% biobased carbon, such as from 50% to 100% biobased carbon, such as from 60% to 100% biobased carbon, such as from 70% to 100% biobased carbon, such as from 75% to 100% biobased carbon, such as from 80% to 100% biobased carbon, such as from 85% to 100% biobased carbon, such as from 90% to 100% biobased carbon, such as from 95% to 100% biobased carbon, such as 100% biobased carbon. In still other embodiments the composition comprises no more than 50% fossil-based carbon, such as no more than 45%, such as no more than 40%, such as no more than 35%, such as no more than 30%, such as no more than 25%, such as no more than 20%, such as no more than 15%, such as no more than 10%, such as no more than 5%, such as no more than 1% fossil-based carbon. In still further embodiments the composition comprises 90% biobased carbon, 91% biobased carbon, 92% biobased carbon, 93% biobased carbon, 94% biobased carbon, 95% biobased carbon, 96% biobased carbon, 97% biobased carbon, 98% biobased carbon, 99% biobased carbon, or 100% biobased carbon, for example 94% biobased carbon. The fermentation composition may further comprise one or more further compounds or metabolites from the cell culture. Such compounds and/or metabolites of the cell culture includes precursors for the metabolite as well as compounds selected from trace metals, vitamins, salts, yeast nitrogen base, carbon source, YNB, and/or amino acids of the fermentation. In particular the composition comprises a concentration of the metabolite of at least 1 mg/kg composition, such as at least 5 mg/kg, such as at least 10 mg/kg, such as at least 20 mg/kg, such as at least 50 mg/kg, such as at least 100 mg/kg, such as at least 500 mg/kg, such as at least 1.000 mg/kg, such as at least 5.000 mg/kg, such as at least 10.000 mg/kg, such as at least 50.000 mg/kg. In other embodiments the composition is substantially free of α-HGA. The composition may also further comprise one or more carriers, agents, additives and/or excipients.

Having generally described this invention, a further understanding can be obtained by reference to certain specific examples, which are provided herein for purposes of illustration only, and are not intended to be limiting unless otherwise specified.

### EXAMPLES

### Materials and Methods

### DE3 Integration

To use pET vectors as expression system, a DE3 cassette containing T7 polymerase was integrated into the genome of DE3 lysogenization kit (Millipore, Damstadt Germany).

### Shake Flask Media and Cultivations

The serine production was checked in M9 minimal media. Glucose M9 minimal media consisted of 2 to 5 g/L glucose, 2 mM Glycine, 0.1 mM CaCl₂), 2.0 mM MgSO₄, 1× trace element solution, and 1×M9 salts. The 1,000× trace element stock solution consisted of 27 g/L FeCl₃*6H₂O, 2 g/L ZnCl₂*4H₂O, 2 g/L CoCl₂*6H₂O, 2 g/L NaMoO₄*2H₂O, 1 g/L CaCl₂)*H₂O, 1.3 g/L CuCl₂*6H₂O, 0.5 g/L H₃BO₃, and concentrated HCl dissolved in ddH₂O and sterile filtered. The 10× M9 salts stock solution consisted of 68 g/L Na₂HPO₄ anhydrous, 30 g/L KH₂PO₄, 5 g/L NaCl, and 10 g/L NH₄Cl dissolved in ddH₂O and autoclaved.

Media was filter sterilized and 50 ml was added to 250 ml sterile baffled shake flask. Overnight cultures were used as inoculum, starting O.D. was 0.1 and until and unless mentioned otherwise, the incubation was at 37 °C and 250 rpm.

### Batch and Fed-Batch Fermentation

Batch and fed batch fermentations were performed in M9-glycine and TPM2 media, respectively, as published previously¹⁵. Except for differences in antibiotics, the media composition and growth and induction conditions were the same. For fed-batch fermentations, a constant or linear feeding rate was followed but was increased stepwise based on the glucose levels in the fermenter, which were monitored by glucose strips.

### Analytical Methods

Glucose, HGA and other organic acids were quantified using previously published HPLC method¹⁶.

The concentration of serine was measured using a Dionex Ultimate 3000 HPLC (High-Performance Liquid Chromatography) equipped with a CHIROBIOTIC® T Chiral (250×2.1 mm×5 μm) column (Sigma-Aldrich, St. Louis, MO, USA) and Diode Array Detector (DAD-UV) detector. The mobile phase comprised of 60% acetonitrile (v/v) and 0.02% (v/v) formic acid in milliQ water. The mobile phase was delivered at a rate of 1.0 mL/min, and the injection volume was kept at 3 μL for standard and all samples. The detection of L-serine was monitored at 205 nm.

### Example 1-Effect of Replacement of NADH Dependent Glyceraldehyde-3-Phosphate Dehydrogenase by NADPH Dependent Glyceraldehyde-3-Phosphate Dehydrogenase

Many NADPH dependent glyceraldehyde-3-phosphate dehydrogenases are reported in literature such as from C. acetobutylicum (gapC)¹⁰ or from Bacillus subtilis (gapB)¹⁷. We have replaced the native gapA with gapC from C. acetobutylicum and monitored hydroxyglutarate production in shake flask and in fed batch fermentation.

Construction of Plasmid Vectors pCDF-serAmut-serC and pACYC-serB

L-serine is produced in E. coli from three enzymes encoded by serA, serB and serC. All genes were isolated from E. coli MG1655 using primers with respective gene names (Table 2). The 100 μl PCR mixture contained 250 nM of each forward and reverse primer, 250 M of dNTPs, 2 U of Phusion polymerase, 1×HF buffer, 1 μl of overnight culture. The following two-step PCR protocol was used for the PCR amplification: An initial denaturation step at 98 °C for 40, followed by 5 cycles of denaturation at 98 °C for 10 seconds, annealing at 55 °C for 30 seconds, extension at 72 °C for 90 seconds, followed by 20 cycles, where the annealing temperature was increased from 55 °C to 65 °C. After column purification, the gene products and plasmids were digested using Fast digest enzymes (Thermoscientific, Waltham, MA, USA). About 500 ng of PCR product or 1 μg of plasmids were subjected to digestion by 1 μl each of the restriction enzymes in 1× fast digest buffer. The reaction was incubated for 3 h and then column purified again. The serA PCR product was subjected to double digestion with NcoI and NotI, while the serC PCR product was digested with NdeI and PacI. pCDF-Duet was first digested with NcoI and NotI for cut and paste cloning of serA leading to plasmid pCDF-Duet-serA. This plasmid was later used for cut and paste cloning of serC thus generating pCDF-Duet-serA-serC. The serB PCR product was cloned in pACYC-Duet vector at NcoI and PacI site leading to pACYC-serB. A typical ligation reaction included 1×T4 ligase buffer 50 ng of plasmid DNA and 100 ng of insert and 0.3 μl/10 μl of T4DNA ligase (Thermoscientific, Waltham, MA, USA).

Feedback inhibition of serA was removed by mutating the three residues H344, N346 and N364 to alanine¹⁸ by site directed mutagenesis (Table 3). The master mix was used as mentioned above with the only modification that the master mix was divided in two equal aliquots, after which forward and reverse primers were added to each aliquot. A total of 100 ng of pCDF-Duet-serA-serC plasmid was used as a template. The two step PCR program: initial denaturation at 98 °C for 40 sec, denaturation at 98 °C for 10 sec, annealing 60 °C for 30 sec, extension 72 °C for 4 min and 30 sec. The cycle was repeated 5 times and then the two aliquots were mixed and redistributed for additional 15 cycles. To enable swapping of vector backbones, the NcoI site inside serC was removed by the same approach using the primers listed in Table 3.

TABLE 2
Primers used for amplification and cloning of serine production pathway

| SEQ ID | Primer name | Sequence |
|---|---|---|
| SEQ ID NO: 67 | serA-NcoI | GGCCCATGGCAAAGGTATCGCTGGAG |
| SEQ ID NO: 68 | serA NotI_R | ATTGCGGCCGCTTAGTACAGCAGACGGGCGCGA |
| SEQ ID NO: 69 | serB_NcoI_F | GGCCCATGGCTAACATTACCTGGTGCG |
| SEQ ID NO: 70 | serB_R Hind | GCCAAGCTTTTATTACTTCTGATTCAGGCTGCC |
| | III | |
| SEQ ID NO: 71 | serB_PacI_R | GCCTTAATTAATTATTACTTCTGATTCAGGCTGCC |
| SEQ ID NO: 72 | serC_F_NdeI | GGCCATATGATGGCTCAAATCTTCAATTTTAGTTCTGG |
| SEQ ID NO: 73 | serC_R_PacI | GCCTTAATTAATCAT TAACCGTGACGGCGTTCGAAC |
| SEQ ID NO: 74 | ydeD_F_Nde | CCGCATATGTCGCGAAAAGATGGGGTG |
| | I | |
| SEQ ID NO: 75 | ydeD_cHis_R | GCCTTAATTAATGATGATGATGATGATGACTTCCC |
| | -PacI | ACCTTTACCGCTTTACGCC |
| SEQ ID NO: 76 | ydeD_NcoI_F | CCGCCATGGCGCGAAAAGATGGGGTG |
| | 1 | |

TABLE 3
Primers used for site directed mutagenesis of serine production pathway

| SEQ ID | Primer name | Sequence |
|---|---|---|
| SEQ ID NO: 77 | serA N364A_F | CGAGCAGGGCGTCGCTATCGCCGCGCAATA |
| SEQ ID NO: 78 | serA N364A_R | TATTGCGCGGCGATAGCGACGCCCTGCTCG |
| SEQ ID NO: 79 | serA H344AN346A F | CTGAT CACATCGCTGAAGCT CGTCCGGGCGTGC |
| SEQ ID NO: 80 | serA H344AN346A_R | GCACGCCCGGACGAGCTTCAGCGATGTGCATCAG |
| SEQ ID NO: 81 | serC_NcoIc_F | CAAGGTATTATTCTGTCATGGCGGTGGTCGCG |
| SEQ ID NO: 82 | serC_NcoIc_R | CGCGACCACCGCCATGACAGAATAATACCTTG |

Replacement of gapA by gapC

gapA was replaced by gapC gene from C. acetobutylicum strain using a cat-sacB based selection system. Cat-sacB was inserted using pKD46 harboring exo, beta and gamma genes for recombination. Positive selection for cassette insertion was done by selecting clones for chloramphenicol resistance. The loss of cassette was selected by replica plating of clones on LB-chloramphenicol and LB-sucrose plate containing 15% sucrose (no NaCl). The cat-sacB cassette was amplified using the primers gapC_camsacB_F and R (Table S3). Apart from template and extension time, the reaction mixture and PCR program was the same as described in example 1. The extension time was 2 min and 30 sec, while the template was 1 μl of overnight culture from E. coli carrying the cat-sacB cassette on its genome. Competent cells were then transformed with 200 ng of gapC-cat-sacB cassette, and were plated on LB-chloramphenicol-ampicillin plates after two hours of regeneration and incubated overnight at 30 °C. A single colony was picked and made electrocompetent after 1 h of induction (Example 1) and transformed with gapC gene which was amplified from the C. acetobutylicim genome using the above mentioned PCR program and primers gapC_aF and aR, as mentioned in Table 4. After two hours of recovery, cells were plated on LB-sucrose plates and incubated at 42 °C to cure pKD46 plasmid. 24 clones of each experiment were replica plated on LB-chloramphenicol and LB plates. Clones that did not grow on LB-chloramphenicol plates were checked for the loss of the cassette by colony PCR and were subsequently sequenced by Sanger sequencing.

TABLE 4
Primers for replacement of gapA by gapC

| SEQ ID | Primer Name | Sequence |
|---|---|---|
| SEQ ID NO: 83 | gapC_camsacB_F | GTAATTTTACAGGCAACCTTTTATTCACTAACAAATAGCTGGTG |
| | | GAATATAAAATGAGACGTTGATCGGCACG |
| SEQ ID NO: 84 | gapC_camsacB_R | AAAAAGAGCGACCGAAGTCGCTCTTTTTAGATCACAGTGTCATC |
| | | TCAACACCAAAGGGAAAACTGTCCATATGCAC |
| SEQ ID NO: 85 | gapC_aF | GTAATTTTACAGGCAACCTTTTATTCACTAACAAATAGCTGGTG |
| | | GAATATATGGCAAAGATAGCTATTAATGG |
| SEQ ID NO: 86 | gapC_aR | AAAAAGAGCGACCGAAGTCGCTCTTTTTAGATCACAGTGTCA |
| | | TCTCAACCTATTTTGCTATTTTTGCAAAGTAAGC |

Subsequently, strains were transformed with pCDF-Duet1-serAmut-serC and pACYC-serB. The resulting glycerol stocks were grown overnight in 2×YT medium containing 0.1% glucose and supplemented with spectinomycin and chloramphenicol. Overnight cultures were inoculated in fed batch fermentation. The media composition and cultivation conditions were as described previously.

### Results

The strain HM_274 does not carry the gapA::gapC replacement. It accumulated 0.91 g/L HGA in 23 h batch fermentation in baffled flask. In a 48 h fed-batch fermentation, the same strain accumulated 9.35 g/L HGA. The strain HM_476 does carry the gapA::gapC replacement. It accumulated 0.86 g/L HGA in 23 h batch fermentation in baffled flask. In a 48 h fed-batch fermentation, the same strain accumulated 2.36 g/L HGA. The data for the 23 h timepoint of the batch fermentation and the 48 h fed-batch fermentation is shown in Table 5. The data clearly demonstrate a reduction in HGA production in a strain where gapA is replaced by gapC.

TABLE 5
Lower HGA accumulation in strain HM_476 wherein
gapA is replaced by gapC compared to HM_274.

| | | HGA [g/L] | HGA [g/L] |
|---|---|---|---|
| Strain | | Batch phase | fermentation |
| Number | Strain genotype | 23 h | 48 h |
| HM_274 | E. coli (DE3) | 0.90965 | 9.35 |
| | pCDF-serAmut-serC | | |
| | pACYC-serB | | |
| HM_476 | E. coli (DE3) gapA::gapC | 0.86125 | 2.36 |
| | pCDF-serAmut-serC | | |
| | pACYC-serB | | |

### Example 2—Effect of Reducing NADH Pool by Oxidation of NADH Using NADH Oxidase (Nox)

### Materials and Methods (Nox)

Construction of Plasmids pSEVA27-serAmut-CB and pSEVA27-serAmut-CB-Nox

All plasmid manipulations were performed using Uracil-Specific Excision Reagent (USER) cloning. PCRs were carried out with Phusion U Hot Start polymerase master mix (Thermo Fisher Scientific, Waltham, MA, USA). Oligonucleotides were ordered from Integrated DNA technologies (IDT, Coralville, IA, USA) and are listed in Table 6. The genes serB and serC were amplified from the E. coli MG1655 genome and cloned into the pSEVA27-sl backbone. serAmut was amplified from pCDFDuet-1-serAmut-serC and cloned into pSEVA27-sl backbone alongside serB and serC. This plasmid was used for cloning nox which was amplified from the Lactobacillus brevis genome. Application of each primer in generating different USER fragments is also mentioned. PCR program: initial denaturation at 98 °C for 40 sec, denaturation at 98 °C for 10 sec, annealing 60 °C for 30 sec, extension 72 °C for 3 min and 30 sec the cycle was repeated 25 times. USER fragments 1 to 4 were used for assembling pSEVA27-serACB while fragments 5 and 6 were used for assembling pSEVA27-serACB-Nox.

The 10 μL USER reaction contained 1 μL of USER enzyme and 1 μL 10× cut smart buffer (New England Biolabs) and 200 ng of each USER fragment. The reaction was incubated at 37 °C for 30 min followed by 15 °C for 30 min. The reaction mixture was transformed in chemically competent NEB5 Alpha cells, transformants were grown in SOC media at 37 °C before plating on LB-kan plates and incubated at 37 °C for overnight.

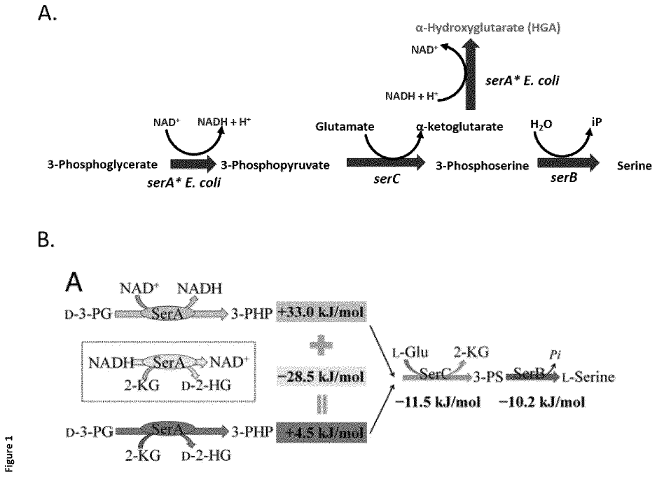

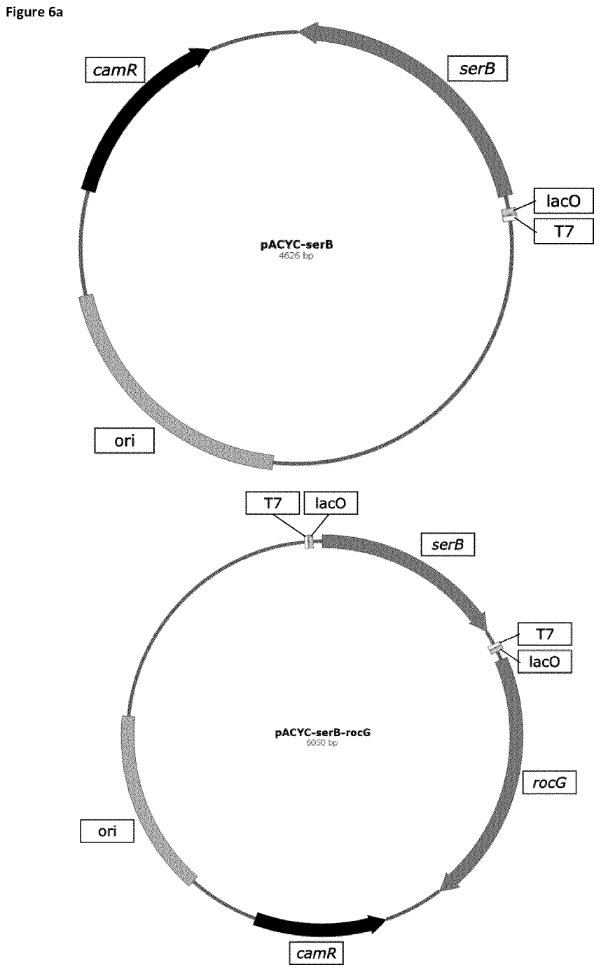

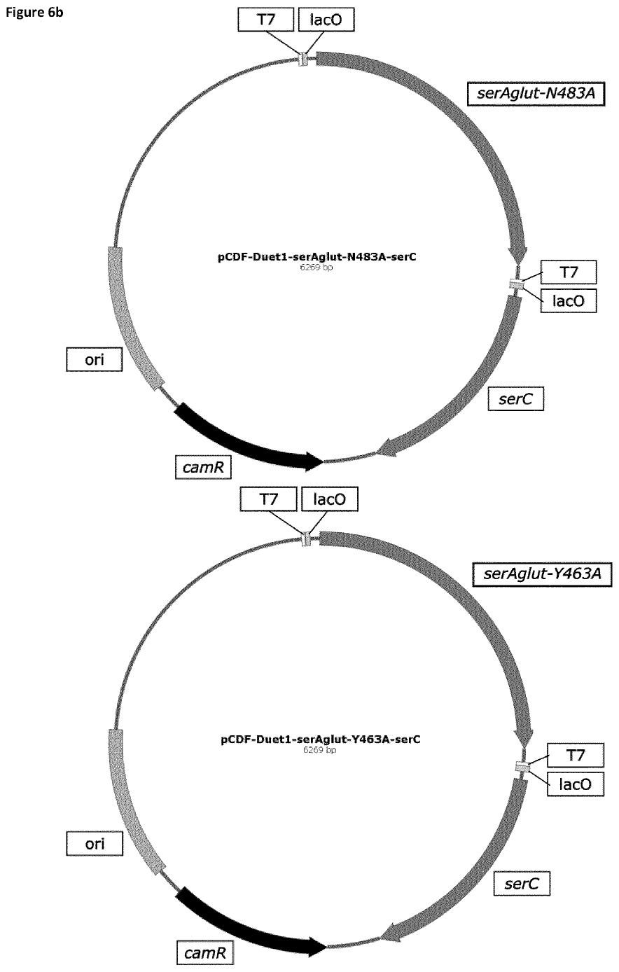

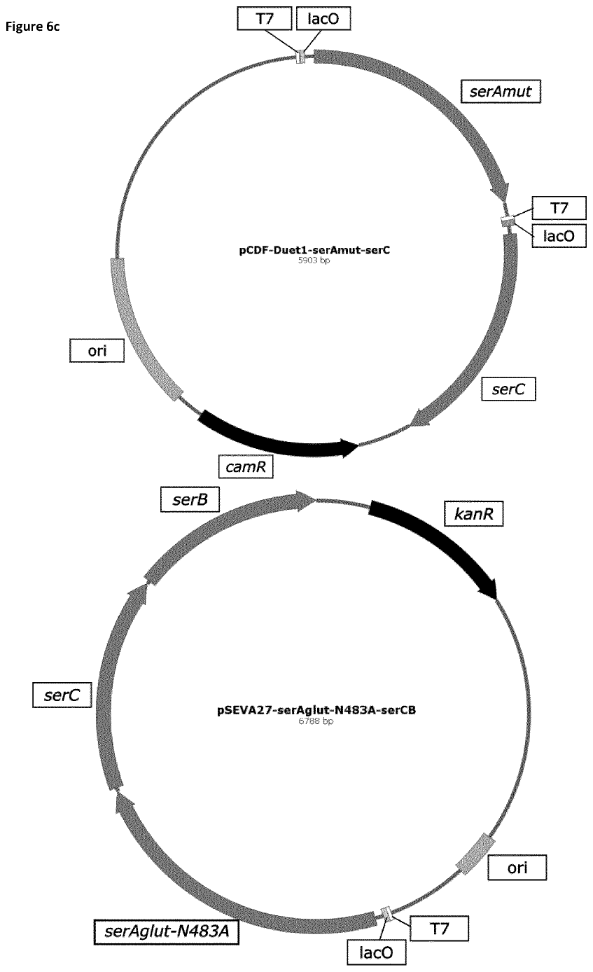

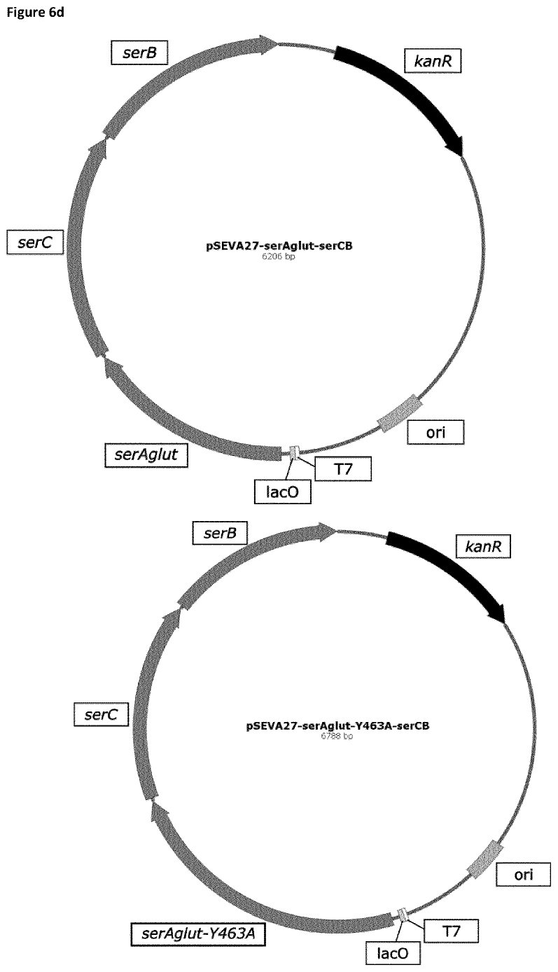

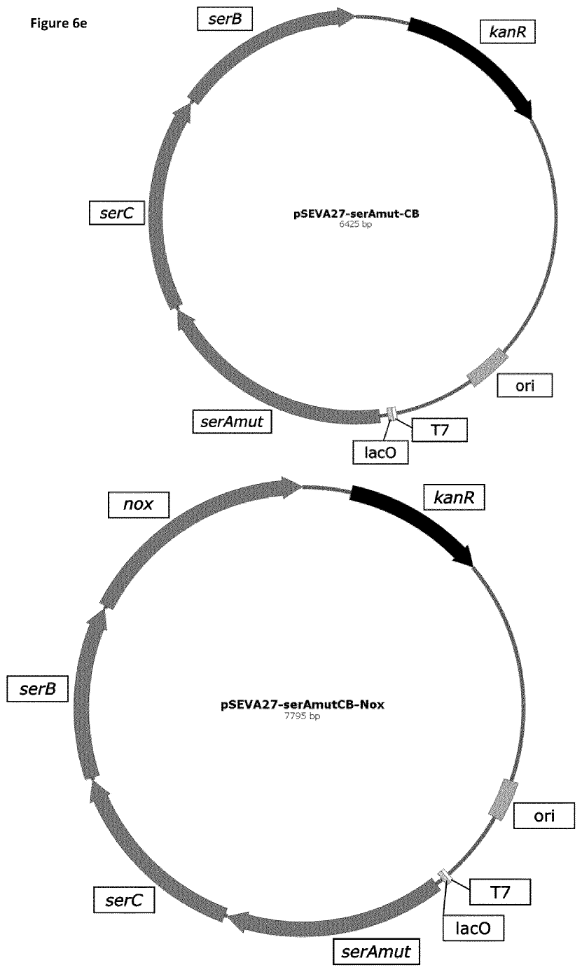

Next day, single colonies were picked and incubated in 2×YT-kan medium and incubated at 37 °C for 16 h for plasmid prep and sanger sequencing. The plasmids maps are given in FIGS. 1 and 6. Confirmed plasmids were transformed in the E. coli strain for L-serine production. The shake flask protocol as described above was used for serine production.

TABLE 6
Primers for used for pSEVA27-serAmut-CB and pSEVA27-serAmut-CB-Nox construction

| | Primer | | |
|---|---|---|---|
| SEQ ID | name | Sequence | Description |
| SEQ ID | pSEVA2 | ATAAGCUGAGGTCGCCTCAGCG | Primer for |
| NO: 87 | 7_fwd | | pSEVA27 serACB- |
| | | | Nox USER |
| | | | fragment 1 |
| SEQ ID | pSEVA2 | AAACTGGTCUCCTTCTTAAAGTTAAACAAAATTATTTCTAGA | Primer for |
| NO: 88 | 7_T7_ | G | pSEVA27 serACB- |
| | rev | | Nox USER |
| | | | fragment 1 |
| SEQ ID | SerA- | AGACCAGTTUATGGCAAAGGTATCGC | Primer for |
| NO: 89 | C_rev | | pSEVA27 serACB- |
| | | | Nox USER |
| | | | fragment 2 |
| | SerA_ | ACCCGGAAUAGCTTTCATTGCCTGC | Primer for |
| | fwd | | pSEVA27 serACB- |
| | | | Nox USER |
| | | | fragment 2 |
| SEQ ID | SerA- | ATTCCGGGUACCATTCGCGCCCGTCTGCTGTACTGATAAAA | Primer for pSEVA |
| NO: 90 | C_fwd | GAAGGAGACCAGTTTATGGCTCAAATCTTCAATTTTAGTTC | 27serACB-Nox |
| | | | USER fragment 3 |
| SEQ ID | SerC- | ATCAACCGUGACGGCGTTCGAACTCAACCATGAAGTCTGTC | Primer for |
| NO: 91 | B_rev | AGCG | pSEVA27 serACB- |
| | | | Nox USER |
| | | | fragment 3 |
| SEQ ID | SerC- | ACGGTTGAUAAAAGAAGGAGACCAGTTTATGCCTAACATTA | Primer for |
| NO: 92 | B_fwd | CCTG | pSEVA27 serACB- |
| | | | Nox USER |
| | | | fragment 4 |
| SEQ ID | serB_UR | AGCTTAUTACTTCTGATTCAGGCTG | Primer for |
| NO: 93 | | | pSEVA27 serACB- |
| | | | Nox USER |
| | | | fragment 4 |
| SEQ ID | serB_No | ATC ACT UCT GAT TCA GGC TGC CTG | Primer for |
| NO: 94 | x_UR | | pSEVA27 serACB- |
| | | | Nox USER |
| | | | fragment 5 |
| SEQ ID | pSEVA_ | ACTGAGGUCGCCTCAGCG | Primer for |
| NO: 95 | Nox_UF | | pSEVA27 serACB- |
| | | | Nox USER |
| | | | fragment 5 |
| SEQ ID | nox_Ib1 | AAGTGAUAA AAGAAGGAGACCAGTTT ATGGAAGTCA | Primer for |
| NO: 96 | 00_UF | CAGTTGTTGG TTG | pSEVA27 serACB- |
| | | | Nox USER |
| | | | fragment 6 |
| SEQ ID | Nox_Ib_ | ACC TCA GUTTATTA AGC GTT AAC TGA TTG GGC | Primer for |
| NO: 97 | UR | | pSEVA27 serACB- |
| | | | Nox USER |
| | | | fragment 6 |

### Results.

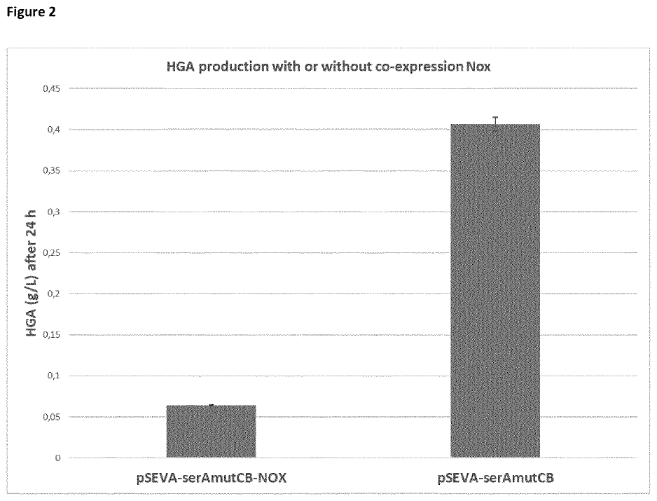

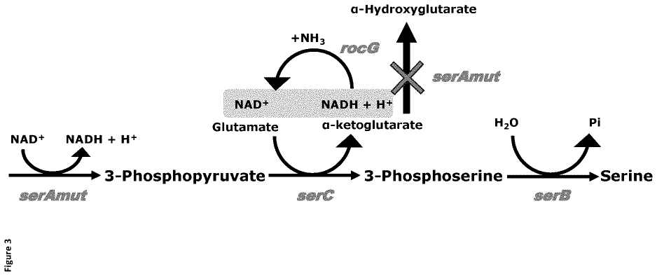

In batch fermentation carried out in baffled flasks, the strain co-expressing Nox and SerACB from pSEVA27-serACB-Nox accumulated 0.06-0.07 g/L HGA after 24 h incubation, as shown in FIG. 2. The strain expressing SerACB only accumulated 0.4 g/L HGA. This data demonstrates that NADH depletion brought by the expression of NADH oxidase leads to reduced HGA production.

### Example 3—Effect of Over Expression of NADH Dependent Glutamate Dehydrogenase

E. coli and C. glutamicum employ NADPH dependent glutamate dehydrogenase reduction of KGA to glutamate to recycle NADPH to NADP+. To switch from NADPH to NADH consumption and thus recycling NADH to NAD+, NADH dependent glutamate dehydrogenases can be expressed. There are many NADH dependent glutamate dehydrogenase known in literature, and we have tested NADH dependent glutamate dehydrogenase RocG from Bacillus subtilis.

### Materials and Methods

rocG was cloned in pACYC-serB plasmid using USER cloning as described above. The primers are given in Table 7a. The PCR program and USER reaction, transformation was performed as described above, the only difference being that the cells were plated on LB-chloramphenicol plate after the regeneration step and 2×YT-chloramphenicol medium was used for cell growth. The medium for production carried out in shake flasks remains as described above.

TABLE 7a
Primers for cloning rocG in pACYC-
serB to constructs pACYC-serB-rocG

| SEQ ID | Primer name | Sequence |
|---|---|---|
| SEQ ID NO: 98 | rocG_UR | AGGTTAUTTAGACC |
| | | CATCCGCGGAAAC |
| SEQ ID NO: 99 | rocG_UF | AGGAGATATACAUATGT |
| | | CAGCAAAGCAAGTCTCG |
| SEQ ID NO: 100 | pACYC_UF | ATAACCUAGGCTGCTGC |
| | | CACCGC |
| SEQ ID NO: 101 | pACYC_UR | ATGTATATCTCCUTCTT |
| | | ATACTTAACTAATATAC |
| | | TAAG |

### Results.

In a 20 h shake flask fermentation, ALE-8 and ALE-8 expressing RocG were monitored regarding the HGA and L-serine concentrations.

TABLE 7b
HGA and serine concentrations detected in supernatants
of a 20 h fed-batch fermentation.

| Strain modification | + rocG | − rocG |
|---|---|---|
| Serine at 20 h (g/L) | 0.37 | 0.74 |
| HGA at 20 h (g/L) | 0.06 | 0.24 |
| Serine/HGA at 20 h | 6.16 | 3.08 |

The strain expressing RocG showed 6 fold reduced HGA concentrations compared to the strain without RocG expression. Though the serine concentrations are also lower, serine/HGA ratio is almost 2-fold better in +rocG strain.

Similarly, in another experiment, ALE-8 accumulated 0.27 g/L HGA 5 h after fermentation start, while ALE-8 expressing RocG accumulated 0.06 g/L HGA. After 48 h, ALE-8 accumulated 0.1 g/L HGA and ALE-8 expressing RocG accumulated 0.06 g/L HGA. After 48 h, ALE-8 had produced 0.75 g/L L-serine and ALE-8 expressing RocG produced 0.3 g/L L-serine.

This experiment shows that the additional expression of rocG helps in reduction of HGA accumulation and its expression also reduces L-serine production. However, if L-serine to HGA ratio is compared for both strain then it is better with rocG expression.

### Example 4—Effect of gapC on L-Serine Production

In this example, we prove that the introduction of gapC increases L-serine production in a strain that expresses serA (E. coli) with KGA reduction activity or in strain which expresses serA with no KGA reduction activity. To demonstrate the first case, the L-serine operon containing feedback insensitive serA from E. coli was transformed in an E. coli strain expressing either gapA or gapC from the genome. To demonstrate the latter case, serA from E. coli was replaced with feedback insensitive serA from C. glutamicum which does not have KGA reduction activity in both pCDF and pSEVA27 vectors.

### Plasmid Construction:

Construction of pCDF-Duet1-serAmut-serC, pACYC-serB and pSEVA27-serAmut-serC has been explained in previous examples. To replace serAmut (E. coli) by serA from Corynebacterium glutamicum in these vectors, the USER primers give in Table 8 were used. The PCR program, USER reaction and transformation were performed as mentioned in above examples. The feedback inhibition was removed by replacing Y463 or N483 to alanine (A) using the site directed mutagenesis protocol explained above and results in vector pCDF-Duet1-serAglut-Y463A-serC and pCDF-Duet1-serAglut-N483A-serC, as well as vector pSEVA27-serAglut-Y463A-serC and pSEVA-serAglut-N483A-serC. Primers are listed in Table 8.

TABLE 8
Primers used for introduction of C. glutamincum serA into vectors pCDF-Duet1-
serAmut-serC, pACYC-serB and pSEVA27-serAmut-serC

| SEQ ID | Primer name | Sequence |
|---|---|---|
| SEQ ID NO: | pCDF_VB_UF | AGCCGCAUAATGCTTAAGTCGAAC |
| 102 | | |
| SEQ ID NO: | pCDF_UR | ATG GTA TAU CTC CTT ATT AAA GTT AAA |
| 103 | | CAA AAT TAT TTC TAC AGG G |
| SEQ ID NO: | serAglut_pCDF_UF | ATATACCAUATGAGCCAGAATGGCCG |
| 104 | | |
| SEQ ID NO: | serAglut_pCDF_UR | ATGCGGCUTTATCAACCAAGCTTGCGAGCC |
| 105 | | |
| SEQ ID NO: | pSEVA_UR | AAACTGGTCUCCTTCTTAAAGTTAAACAAA |
| 106 | | |
| SEQ ID NO: | pSEVA_UF | AGCTTGGTUGATAAAAGAAGGAGACCAGTTTATGGCTCAAATCTTC |
| 107 | | |
| SEQ ID NO: | Cglut_pSEVA_UF | AGA CCA GTT UATG AGC CAG AAT GGC CGT CC |
| 108 | | |
| SEQ ID NO: | Cglut_pSEVA_UR | AAC CAA GCUTTA TTA GTC AAG ATC AAC CTG GAA GG |
| 109 | | |
| SEQ ID NO: | CglutserA | CTG CTG GCA TCG CCA TCG AGG CTG |
| 110 | N483A_F | |
| SEQ ID NO: | CglutserA | CAG CCT CGA TGG CGA TGC CAG CAG |
| 111 | N483A_R | |
| SEQ ID NO: | CglutserA Y463A_F | CTC TTC CTG CAG GCC ACT GAC GCT C |
| 112 | | |
| SEQ ID NO: | CglutserA | GAG CGT CAG TGG CCT GCA GGA AGA G |
| 113 | Y463A_R | |

### Results

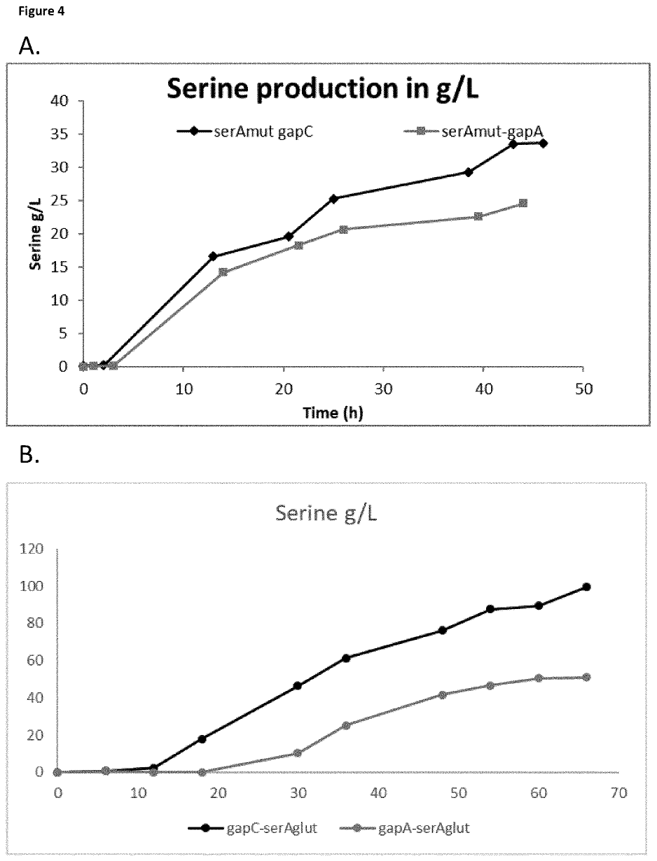

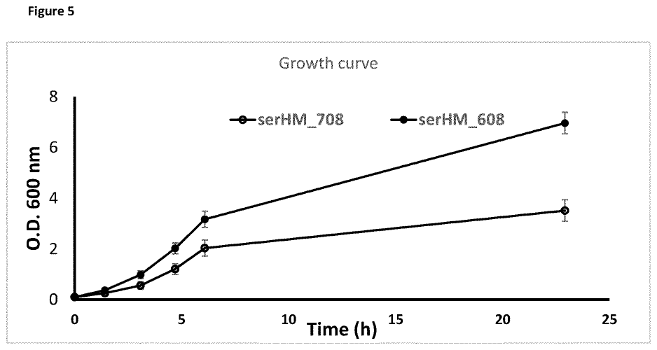

L-serine production form strains with either gapA or gapC on the genome expressing feedback inhibition insensitive serA from E. coli were compared in fed-batch fermentation over the course of 48 h. The L-serine concentrations measured during the fermentation are shown in FIG. 4A. The strain expressing gapA produced 25 g/L, while the strain expressing gapC produced 35 g/L. Furthermore, L-serine production form strains with either gapA or gapC on the genome expressing feedback inhibition insensitive serA from C. glutamicum were compared in fed-batch fermentation over the course of 68 h. The L-serine concentrations measured during the fermentation are shown in FIG. 4B. The strain expressing gapA produced 50 g/L, while the strain expressing gapC produced 100 g/L.

Firstly, this data demonstrates that a 40% increase in L-serine production in fermentation can be achieved from a strain wherein gapA is replaced by gapC and which expressed the L-serine pathway including E. coli serA with promiscuous activity for KGA. Secondly, the gapA::gapC modification boosts L-serine production by 200% for strains expressing serine pathway including C. glutamicum serA with no promiscuous activity for KGA.

### Example 5—The Effect of gapC on the Strain Expressing serA with No HGA Activity

In this example, we demonstrate that enhancing the NADPH pool expressing for instance gapC not only enhances L-serine production (Example 4), it also reduces the redox imbalance caused by expression of serA from C. glutamicum which cannot oxidize NADH while reducing KGA to HGA. Additional NADPH supply (from gapC in this example) in turn likely supplies the NADPH required for recycling of KGA to Glutamate. To demonstrate this, we expressed the L-serine pathway from the vectors pCDF and pACYC containing serA with no HGA activity such as from C. glutamicum in both gapA (serHM_708) and gapC (serHM_608) strains. The two strains serHM_708 and serHM_608 were compared in a batch fermentation experiment.

As shown in FIG. 5, the gapC strain (serHM_608) grows to a final OD of 7, while the gapA strain (serHM_708) grows to a final OD of 3.

### Example 6—The Effect of Rat serA in the Strain Expressing gapC

The serA gene from rat is a Type I serA and is found to have negligible activity for production of hydroxyglutarate. The gene was ordered as gene fragment from Twist Biosicence (USA). This serA is disclosed herein as SEQ ID NO: 8. To clone rat serA, the pSEVA serACB vector with constitutive promoter strength was chosen. These vectors are well known in the art. Vector backbone primers oSER_1424 and oSER_1426 bind the sequence upstream and downstream of the serA being replaced. The gene fragment with complementary USER overhangs were amplified using primers oSER_1423 and oSER_1425.

PCR program: initial denaturation at 98 °C for 40 sec, denaturation at 98 °C for 10 sec, annealing 60 °C for 30 sec, extension 72 °C for 3 min and 30 sec (30 sec/kb) the cycle was repeated 25 times. The primer sequences are given in table below.

TABLE 9
Primers used for introduction of rat serA (SEQ ID NO: 8
and its truncated version (encoded by SEQ ID NO: 144) in the
pSEVA plasmid with constative expression of serine pathway.

| SEQ ID | Primers | oSER name | Sequence | Description |
|---|---|---|---|---|
| SEQ ID NO: | serA_rat_UF | OSER_1423 | agtcctaggUataatgctagctc | Amplification of serA rat |
| 120 | | | ttaatcatgctaaggagg | |
| SEQ ID NO: | serA_rat_U | OSER 1425 | attgaagatUtgggccatGTG | Amplification of serA rat |
| 121 | R | | GAAgtctc | |
| SEQ ID NO: | serA_rat_UF | OSER_1424 | acctaggacUgagctagctgtca | Amplification of plasmid |
| 122 | pBB | | aAggcctatg | for cloning serA rat |
| SEQ ID NO: | serA_rat_U | OSER 1426 | aatcttcaaUtttagttctggtcc | Amplification of plasmid |
| 123 | R_pBB | | ggcaatgc | for cloning serA rat |
| SEQ ID NO: | trunc- | OSER 1423 | agtcctaggUataatgctagctc | Amplification of |
| 124 | serA_rat_Uf | | ttaatcatgctaaggagg | truncated serA rat |
| SEQ ID NO: | trunc- | OSER 1535 | aaccaagcUttaTTCAGCTA | Amplification of |
| 125 | serA_rat_U | | ATCCAATCCAAGGCTTGG | truncated serA rat |
| | R | | | |
| SEQ ID NO: | trunc- | OSER_1424 | acctaggacUgagctagctgtca | Amplification of plasmid |
| 126 | serA_rat_UF | | aAggcctatg | for cloning truncated |
| | _pBB | | | serA rat |
| SEQ ID NO: | trunc- | OSER_1533 | agcttggtUgataaaagaagga | Amplification of plasmid |
| 127 | serA_rat_U | | gacTTCC | for cloning truncated |
| | R_pBB | | | serA rat |

The 10 μL USER reaction contained 1 μL of USER enzyme and 1 μl 10× cut smart buffer (New England Biolabs) and 200 ng of each USER fragment. The reaction was incubated at 37 °C for 30 min followed by 15 °C for 30 min. The reaction mixture was transformed in chemically competent NEB5 Alpha cells, transformants were grown in SOC media at 37 °C before plating on LB-kan plates and incubated at 37 °C for overnight.

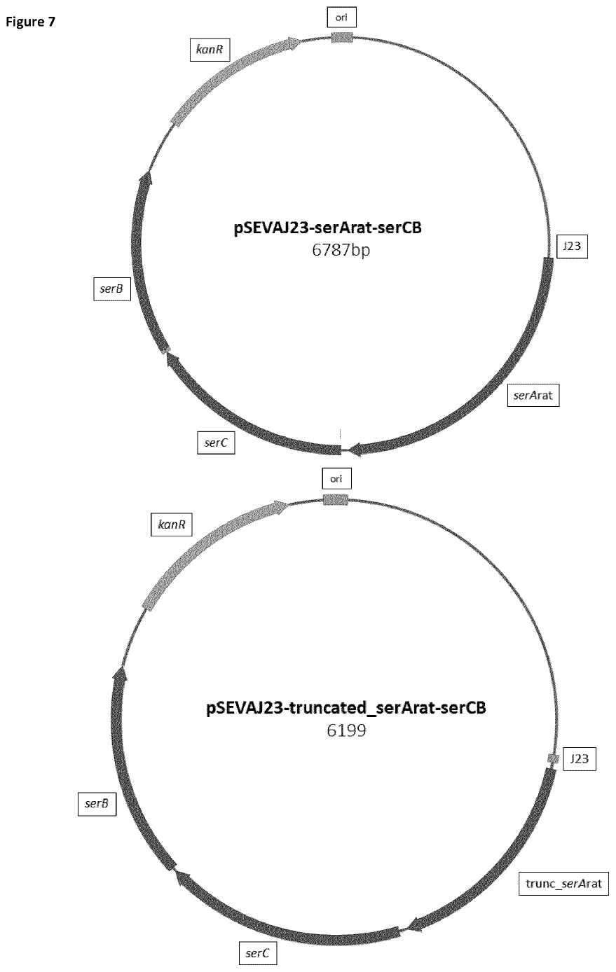

Next day, single colonies were picked and incubated in 2×YT-kan medium and incubated at 37 °C for 16 h for plasmid prep and sanger sequencing. The plasmids maps are given in FIG. 7. Confirmed plasmids were transformed in the E. coli strain for L-serine production. The truncated version of rat serA was obtained by using primers oSER_1424 and oSER_1533. The above obtained plasmid was used as a template. The PCR, USER reaction and transformation protocol as same as above. The shake flask media and protocol are described below.

The growth and L-serine titers in production strains transformed with different serA variants was tested on a 24-hour shake flask experiment. To this end, MOPS-base medium (pH 7.6) was prepared by adding 10 g/L of ammonium sulphate (CAS No. 7783-20-2), 2 g/L of monopotassium phosphate (CAS No. 7778-77), 2 g/L of yeast extract, 40 g/L of MOPS (CAS No. 1132-61-2), and 0.6 g/L of glycine to the desired total medium volume. The medium was adjusted to pH 7.6 using a 15% NH₃ solution and autoclaved. Subsequently, 20 mL of the sterile MOPS-base medium were added to a 250-mL sterilized shake flask and supplemented with 600 μL of glucose monohydrate (CAS No. 14431-43-7), 80 μL of trace elements, and 200 UL of magnesium sulfate heptahydrate (CAS No. 56-40-6). Then, the OD₆₀₀ of overnight cultures was measured to use them as inoculum. From the strain with the lowest optical density, 1 mL was inoculated into the shake flask, whereas the remaining strains were inoculated with correspondingly less volume, leading to the same starting biomass in all shake flasks. These were incubated at 37 °C and 250 rpm for 24 hours. The optical density was monitored after 1.5, 3, 4.5, 6, 7.5 and 24 hours of growth. At the same time points, 300 μl of the culture were sampled into a 96-deep well plate, span down at 3,500×g and 4 °C for 5 minutes, and resulting supernatants were filtered through a 22-μm membrane into a HPLC plate for L-serine later quantification.

TABLE 10
The strain numbers and the brief description
of the variables in each construct.

| Strain | Description |
|---|---|
| SER_1739 | gapC-serA_rat |
| SER_1827 | gapC-trunc_serA_rat |
| SER_351 | gapC-serA_Cglutamicum |

### Results

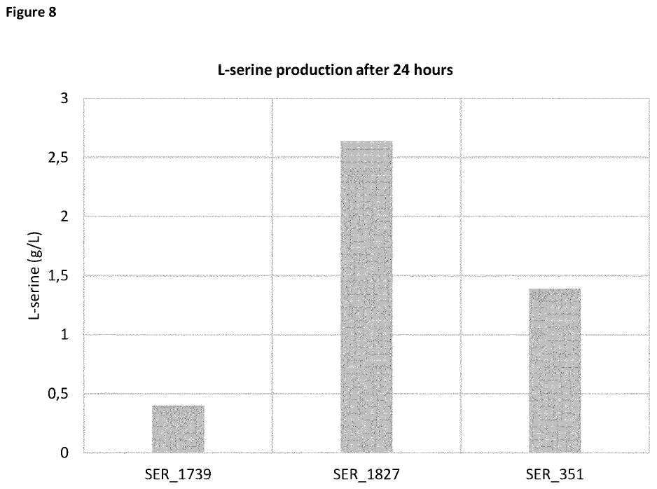

As shown in FIG. 8, the strain expressing serA from rat yielded surprisingly low titers as it is found not to be inhibited by L-serine. However, upon removing the regulatory domain by using primers oSER_1423 and oSER_1535, the truncated variant of the serA rat gene (SEQ ID NO: 144/SER_1827) produced more L-serine, reaching 2.6 g/L after 24 hours. The amount of L-serine produced by the truncated serA rat variant (SER_1827) was surprisingly higher compared to the untruncated (SER_1739) and control (SER_351) strains, which after 24 hours produced 0.4 and 1.4 g/L of L-serine, respectively. This shows that strains employing truncated serA from Type I classification can produce significant amounts of L-serine.

### Example 7—Effect of Replacement of NADH Dependent Glyceraldehyde-3-Phosphate Dehydrogenase by Variants of NADPH Dependent Glyceraldehyde-3-Phosphate Dehydrogenase

Many different variants of NADPH glyceraldehyde 3 phosphate dehydrogenase are known. Some of them are shown in SEQ ID NO: 23 to 46. In order to prove that NADPH dependent glyceraldehyde-3-phosphate dehydrogenase can also be used for L-serine production, the synthetic gene fragments of SEQ ID NO: 25 and 36 with additional 500 bp overhangs complementary to upstream and downstream region of gapA were ordered from twist bioscience (USA). The cat-sacB selection system mentioned in Example 1 was used to replace gapA. The primers used to amplify the gene fragment replacing cat-sacB cassette at gapA site is given in table below.

TABLE 11
Primers used for amplification of gap variants replacing
cat-sacB cassette integrated at gapA site

| SEQ ID | Primers | oSER name | Sequence | Description |
|---|---|---|---|---|
| SEQ ID | gap_Cglit_F | OSER 1109 | acagccggaatca | Amplification of gapB from |
| NO: 128 | | | tacttggtttgg | C. glutamicum |
| SEQ ID | gap_Cglut_R | OSER_1118 | aaattcgccatgt | Amplification of gapB from |
| NO: 129 | | | gattcaagatcg | C. glutamicum |
| SEQ ID | gap_Klactis_F | OSER 1109 | acagccggaatca | amplification of gpd1 from |
| NO: 130 | | | tacttggtttgg | K. lactis |
| SEQ ID | gap_Klactis_R | OSER 1118 | aaattcgccatgt | amplification of gpd1 from |
| NO: 131 | | | gattcaagatcg | K. lactis |

The strains were transformed with pSER_43 plasmid. The plasmid contains strong constitutive promoter expressing serine operon with feedback insensitive serA from C. glutamicum (SEQ ID NO: 14). The transformants were selected on kanamycin plate. The inoculation and shake flask protocol for L-serine production is as mentioned in Example 6.

### Results

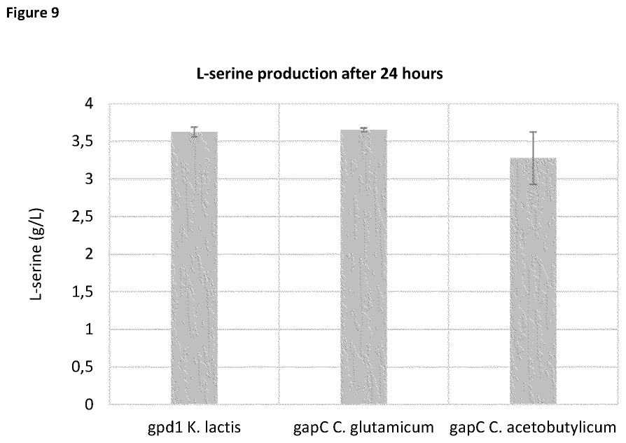

As shown in FIG. 9, the NADPH glyceraldehyde 3 phosphate dehydrogenase from C. glutamicum and K. lactis, with average titres of 3.62 and 3.65 g/L were on par with L-serine production from gapC from C. acetobutylicum, which produced on average 3.3 g/L of L-serine after 24 hours. Hence, these variants can also be used for enhancing NADPH pool.

### Example 8—Effect of Reducing NADH Pool on Production of L-Serine by Oxidation of NADH Using Various Variants of NADH Oxidase

In Example 2, the effect of NADH oxidase from Lactobacillus brevis (seq ID 49) on reducing hydroxyglutarate was demonstrated. In this example, various Nox variants (SEQ ID NO: 50, 52 and 55) downstream of serACB operon of serA from C. glutamicum (non-HGA producing) were cloned. In this example the aim was to analyze if expression of other variants of NADH oxidase would enhance the L-serine production.

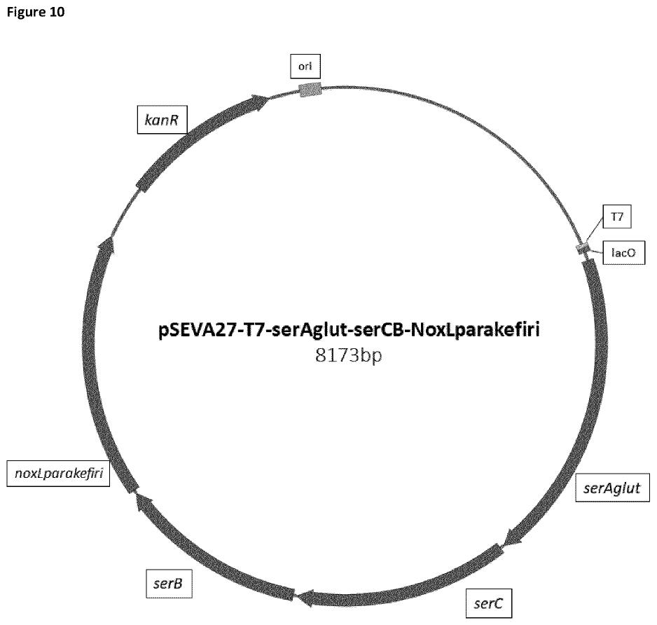

The gene fragments were ordered from Twist Bioscience (SEQ ID NO: 50, 52, 55). The strategy for cloning was the same as mentioned in Example 2. The primers used for amplification of different Nox variants are shown in table below. The constructs are shown in FIG. 10. The inoculation and shake flask experiments were performed as in example 6 and 7. The only difference being that the culture was induced with addition of 40 μM (final concentration) IPTG at OD₆₀₀ values between 0.55 and 0.65.

TABLE 12
Primers used for cloning Nox gene downstream
of serACB operon under T7 promoter.

| SEQ ID | Primers | oSER name | Sequence | Description |
|---|---|---|---|---|
| SEQ ID | nox_UF | OSER 1419 | agtcaccaUccgt | amplification |
| NO: 132 | | | cacgctgacctg | of nox |
| SEQ ID | nox_UR | OSER_1421 | atggtgacUtccg | amplification |
| NO: 133 | | | ccttttcattcac | of nox |
| | | | ttttgg | |
| SEQ ID | nox_UF_ | OSER 1420 | atggtgacUtccg | Plasmid backbone |
| NO: 134 | pBB | | ccttttcattcac | amplification for |
| | | | ttttgg | cloning of nox |
| | | | | genes |
| SEQ ID | nox_UR_ | OSER_1422 | agatccagUaatg | plasmid backbone |
| NO: 135 | pBB | | acctcagaactcc | amplification for |
| | | | atctgg | cloning of nox |
| | | | | genes |

TABLE 13
Strain numbers and the brief description
of the variables in each construct

| Strain | Description |
|---|---|
| SER_1888 | gapC-serA_C. glutamicum-nox_L. parakefiri |
| SER_1911 | gapC-serA_C. glutamicum |

### Results:

As shown in FIG. 11, expression of Nox from L. parakefiri (SER_1888) led to a L-serine titer of 1.9 g/L after 24 hours, therefore higher than the amount produced by the control strain (SER_1911), which reached 1.6 g/L after 24 hours. This indicates that expression of NADH oxidase from L. parakefiri helps enhancing L-serine production.

### Example 9—Effect of Increasing NADPH Pool by Over Expression of Genes from Pentose Phosphate Pathway

The pentose phosphate pathway is one of the main sources of providing NADPH pool to bacteria. In this example it was tested if increasing NADPH pool by over-expressing zwf which encodes for Glucose 6 phosphate dehydrogenase or pgl which encodes for 6-phosphogluconolactonase in the pentose phosphate pathway, could enhance the NADPH pool inside the cells. In this example the effect of these enzymes in presence and absence NADPH-based glyceraldehyde phosphate dehydrogenase (gapC) was studied. Zwf and pgl were amplified from MG1655 genome using the primers mentioned in table below.

PCR program: initial denaturation at 98 °C for 40 sec, denaturation at 98 °C for 10 sec, annealing 60 °C for 30 sec, extension 72 °C for 1 min the cycle was repeated 25 times.

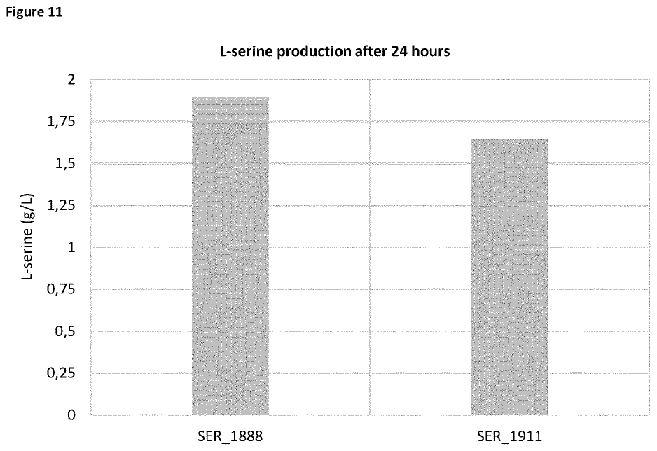

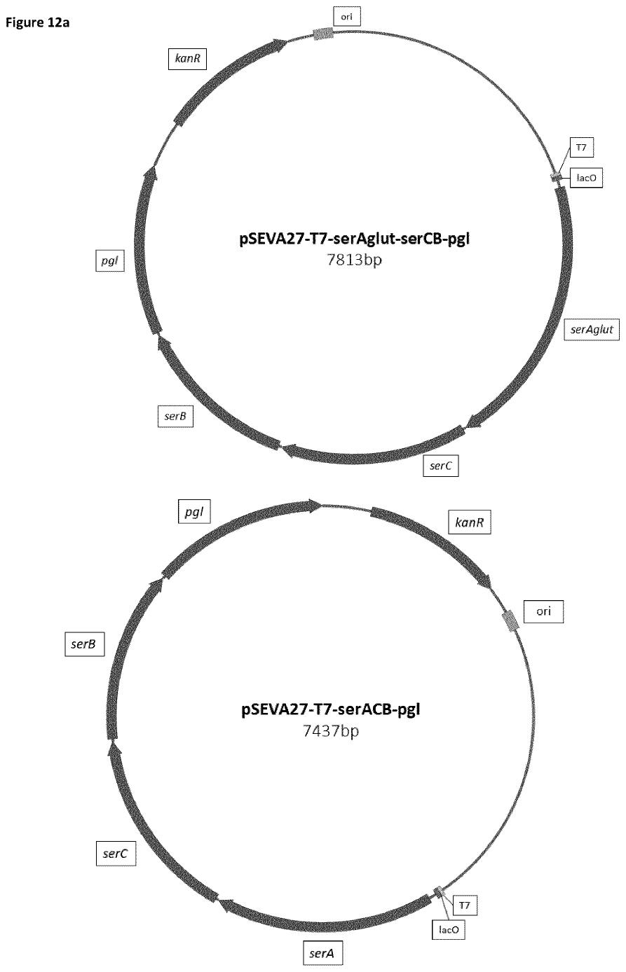

Like NADH oxidase, the zwf and pgl genes were cloned below serB in serACB operon. Each gene was cloned in an operon containing either feedback insensitive serA from E. Coli (to check for reduction in production of hydroxyglutarate) or serA from C. glutamicum (to check if the production leads to increase in L-serine production). The constructs are shown in FIG. 12.

TABLE 14
Primers used for cloning pgl and zwf genes downstream of the serACB
operon under T7 promoter.

| SEQ ID | Primers | oSER name | Sequence | Description |
|---|---|---|---|---|
| SEQ ID | zwf_UF | OSER_1606 | agaccagttUatggcggtaacgcaaacagc | amplification of |
| NO: | | | | zwf |
| 136 | | | | |
| SEQ ID | zwf_UR | OSER_1607 | acctcagcUtactcaaactcattccaggaacgacc | amplification of |
| NO: | | | | zwf |
| 137 | | | | |
| SEQ ID | zwf_UF_pBB | OSER 379 | agctgaggUcgcctcagc | plasmid |
| NO: | | | | backbone |
| 138 | | | | amplification |
| | | | | for cloning of |
| | | | | zwf genes |
| SEQ ID | zwf_UR_pBB | OSER_1608 | aaactggtcUccttcttttatcacttctgattc | plasmid |
| NO: | | | | backbone |
| 139 | | | | amplification |
| | | | | for cloning of |
| | | | | zwf genes |
| SEQ ID | pgl_UF | OSER 1604 | agaccagttUatgaagcaaacagtttatatcgccagc | amplification of |
| NO: | | | | pgl |
| 140 | | | | |
| SEQ ID | pgl_UR | OSER 1605 | acctcagcUtagtgtgcgttaaccaccaccc | amplification of |
| NO: | | | | pgl |
| 141 | | | | |
| SEQ ID | pgl_UF_pBB | oSER_379 | agctgaggUcgcctcagc | plasmid |
| NO: | | | | backbone |
| 142 | | | | amplification |
| | | | | for cloning of |
| | | | | pgl genes |
| SEQ ID | pgl_UR_pBB | OSER_1608 | aaactggtcUccttcttttatcacttctgattc | plasmid |
| NO: | | | | backbone |
| 143 | | | | amplification |
| | | | | for cloning of |
| | | | | pgl genes |

The cloning strategy and shake flask study protocol is same as in Example 8.

TABLE 15
Strain numbers and the brief description
of the variables in each construct

| Strain | Description |
|---|---|
| SER_1880 | gapC-serA_C. glutamicum-pgl |
| SER_1881 | gapC-serA_E. coli-pgl |
| SER_1882 | gapC-serA_C. glutamicum-zwf |
| SER_1884 | gapC-serA_E. coli-zwf |
| SER_1890 | gapA-serA_C. glutamicum-pgl |
| SER_1891 | gapA-serA_E. coli-pgl |
| SER_1892 | gapA-serA_C. glutamicum-zwf |
| SER_1893 | gapA-serA_E. coli-zwf |
| SER_1901 | gapA-serA_C. glutamicum |
| SER_1902 | gapA-serA_E. coli |
| SER_1911 | gapC-serA_C. glutamicum |
| SER_1912 | gapC-serA_E. coli |

### Results

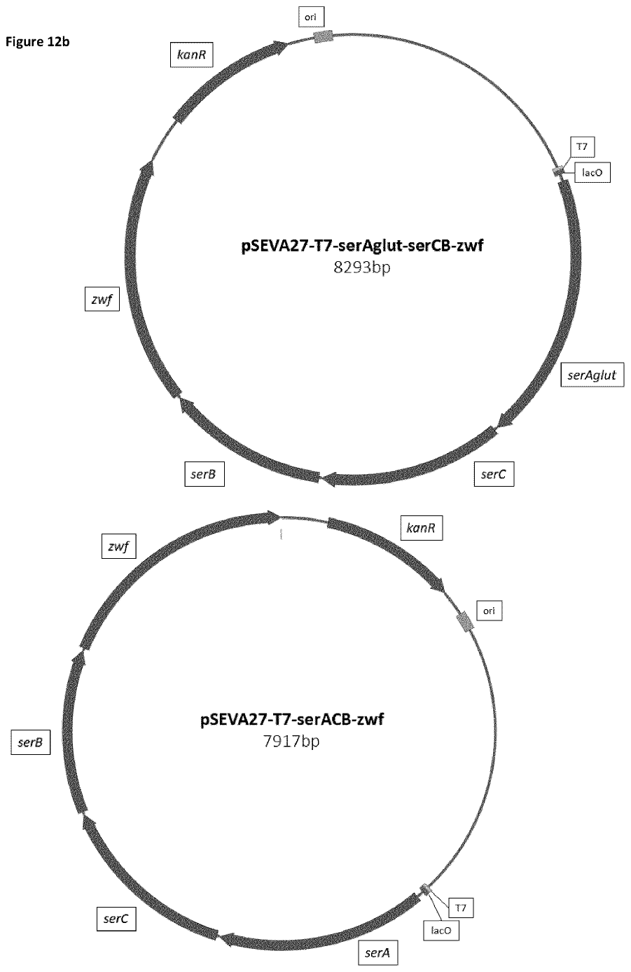

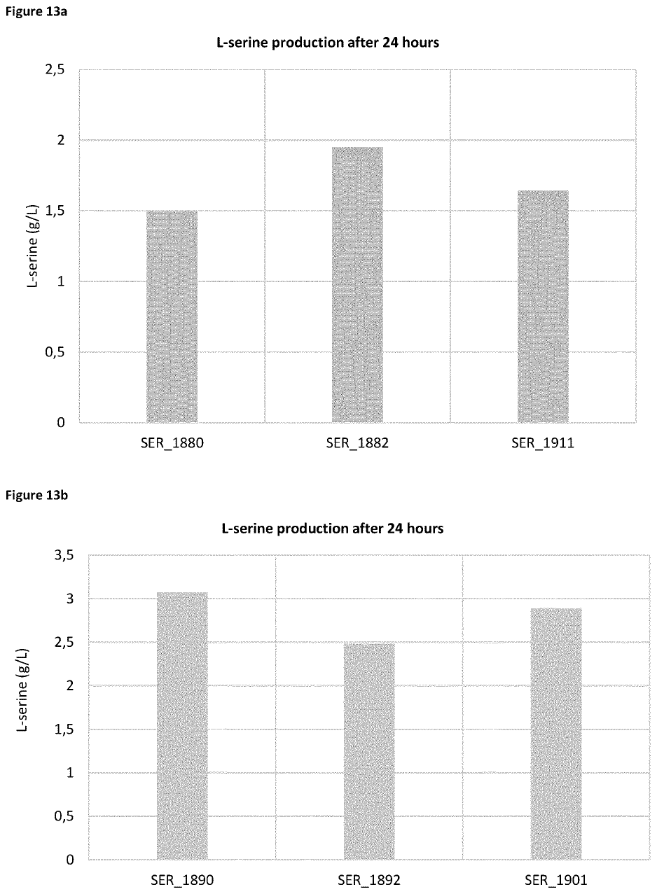

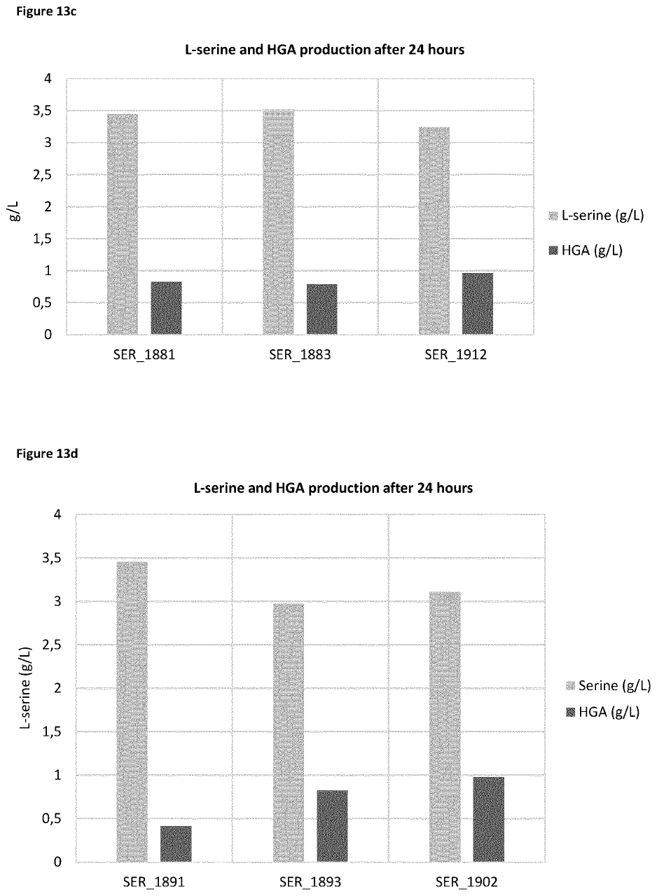

The over expression of gpl and zwf genes influenced L-serine production when combined with serA from C. glutamicum. As can be seen in FIG. 13a, over-expression of zwf in a gapC background (SER_1882) led to a L-serine production of 1.95 g/L, therefore higher than 1.6 g/L of L-serine produced by the control strain (SER_1911). Furthermore, when pgl (SER_1890) and zwf (SER_1892) were over expressed with serA from C. glutamicum in a gapA background, pgl led to enhanced L-serine production, yielding titers of 3.1 g/L. As observed in FIG. 13b, this L-serine amount was higher than the produced by the control strain (SER_1901), which achieved a production of 2.9 g/L of L-serine after 24 hours.

Likewise, over-expression of pgl and zwf had an effect on L-serine and hydroxyglutarate production when combined with serA from E. coli. As displayed in FIG. 13c, strains with a gapC background expressing pgl (SER_1881) and zwf (SER_1883) increased L-serine production, with titres of 3.4 and 3.5 g/L, respectively, whereas the control strain (SER_1912) produced 3.2 g/L of L-serine after 24 hours. Moreover, HGA production was diminished from 0.96 g/L in the control strain, to 0.83 and 0.79 g/L when over-expressing pgl and zwf, respectively. Similarly, over-expression of pgl (SER_1891) and zwf (SER_1893) in combination with serA from E. coli in gapA strains led to reduced HGA production. As shown in FIG. 13d, HGA was reduced from 0.98 g/L in the control strain (SER_1902) to 0.41 and 0.82 g/L when over-expressing pgl and zwf, respectively. In addition, over-expression of pgl in such gapA background led to improved L-serine production, reaching 3.45 g/L after 24 hours. This strain produced more L-serine amount than the control strain, which yielded 3.1 g/L at the same timepoint.

Hence, over-expression of pgl and zwf enhances L-serine production and reduces HGA production. Such effects are more pronounced in gapA strains.

### Example 10—Motifs of PGDH Having Reduced HGA Activity

Some PGDHs can use α-ketoglutarate (KG) instead of PHP in the reverse direction to produce α-hydroxyglutarate. Three enzymes have been found to be able to utilize αKG as a substrate: PDGH from E. coli (REF), Pseudomonas stutzeri (REF), and Saccharomyces cerevisiae (REF). These three enzymes are all type II PGDH's, and it is hypothesized that type I and type III PGDH's may not be able to use αKG as a substrate and that the ability to use αKG as a substrate is a trait specific to type II PGDH's.

To examine conserved residues in type I and II PGDH's, a representative from each class was selected. For type I, PGDH from M. tuberculosis was selected whereas PGDH from E. coli was selected for type II. These enzymes were selected because they are extensively studied, and crystal structures in complex with a relevant substrate exist for both. Residue conservation was examined using the ConSurf-DB server. The relevant crystal structures were used as input; type I (M. tuberculosis, PDB ID: 3DDN) and type II (E. coli, PDB ID: 1YBA). The ConSurf workflow was as previously described. Briefly, amino acid sequences similar to each sequence in the PDB ID provided were collected and multiply aligned using HMMER and MAFFT, respectively. The evolutionary conservation of each amino acid position in the alignment was calculated using the Rate4Site algorithm, implemented in the ConSurf web-server. The algorithm took explicitly into account the phylogenetic relations between the aligned proteins and the stochastic nature of the evolutionary process. Rate4Site assigned a conservation level for each residue using an empirical Bayesian inference. The continuous conservation scores were divided into a discrete scale of nine grades for visualization (not shown), from the most variable positions (grade 1) coloured turquoise, through intermediately conserved positions (grade 5) coloured white, to the most conserved positions (grade 9) coloured maroon. The conservation scores were projected onto the protein/nucleotide sequence and on the crystal structure corresponding to the PDB ID used as input.

### Results

Inspection of the conserved amino acid sequences for type I and type II of PGDH showed that several regions facing the active site are conserved in both type I and II PHDG's. One such motif is the G/ARAGV and GCFCI motif in type I and II PGDHs respectively. One distinct, and highly conserved, difference between these two motives was the residue type at the second position. In type I PGDHs this is an arginine residue whereas it is most often a cysteine residue in Type II enzymes. Inspection of a representative type I (M. tuberculosis) and II (E. coli) PGDH structure with PHP and αKG bound, respectively, shows that both the arginine and cysteine residues are facing the active site, suggesting these residues could be involved in controlling substrate specificity, and hence the ability to use αKG as a substrate. This was further investigated using site-directed mutagenesis to substitute the arginyl side chain in M. tuberculosis PGDH with other selected amino acid side chains such as alanine and leucine. Removal of the cationic group of Arg 72 in M. tuberculosis PGDH converts the enzyme from one that does not accept αKG as a substrate to one that does. However, replacement of the arginyl side chain with a lysyl side chain failed to accomplish this. These results clearly show that it is the presence of a cationic side chain at the second position in the conserved motif which renders the enzyme unable to use αKG.

### Example 11—Effect of NADH-Dependent Glyceraldehyde-3-Phosphate Dehydrogenase Replacement by NADPH-Dependent Glyceraldehyde-3-Phosphate Dehydrogenase on Intracellular NAD(P)H Pool

In this example, it is demonstrated that the introduction of gapC (SEQ ID NO: 43) or gdp1 (SEQ ID NO: 36) increases the overall NADP(H) pool in a strain that expresses serA (C. glutamicum/SEQ ID NO: 14) with no KGA reduction activity. To demonstrate this, E. coli strains expressing either gapA, gapC or gdp1 from the genome were transformed with pSER_43 plasmid. This plasmid contains strong constitutive promoter expressing the serine operon with serA from C. glutamicum (SEQ ID NO: 14). The transformants were selected on kanamycin plate. The inoculation and shake flask proceeded as described in Example 6. After 24 hours of incubation, for each strain, 1 ml of biomass was sampled and quenched with 1 mL of 40% ethanol-0.8% sodium chloride solution, followed by submersion of the samples for 20 seconds in a dry ice-ethanol bath. Then, samples were incubated on ice for 15 minutes, and centrifuged at 11,000×g for 5 minutes at 4 °C. Cell pellets were resuspended in 1 mL of MQ water. Quantification of the intracellular NADP(H) pool was achieved using the [NADP⁺]/[NADPH] Quantification Kit (Sigma-Aldrich) according to the manufacturer's instructions.

### Results

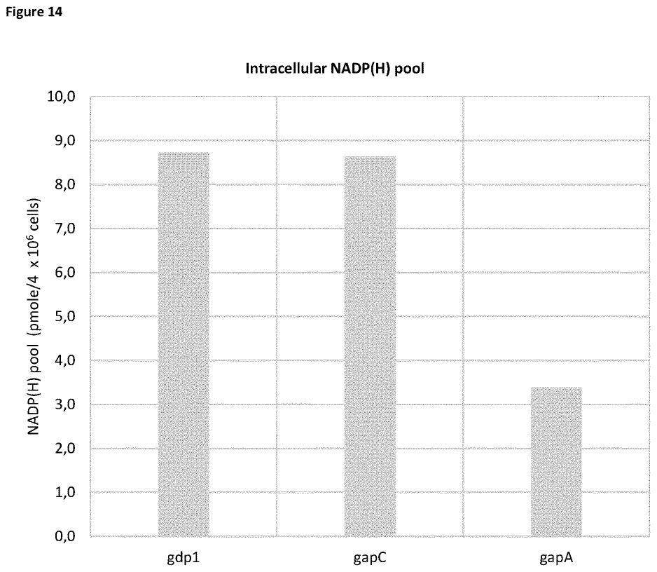

As shown in FIG. 14, the replacement of NADH-dependent glyceraldehyde-3-phosphate dehydrogenase (gapA) by a NADPH-dependent glyceraldehyde-3-phosphate dehydrogenase (gapC and gdp1) when expressing serA with no KGA reducing activity leads to an increased overall intracellular cofactor pool. The effect of gdp1 and gapC on the NADP(H) pool are comparable. Results are expressed as the intracellular concentration of NADP⁺ and NADPH (pmole) in 4×10⁶ cells.

Overall this example also shows that combining (over) expression of a NADPH producing GAPDH, in particular GapC, and a non-HGA producing PGDH, in particular serA from C. glutamicum balances the NAPDH and the NADPH pools in the cell and allows for superior production of L-serine.

### LIST OF REFERENCES CITED IN THE DESCRIPTION

1. Grant, G. A. D-3-phosphoglycerate dehydrogenase. Front. Mol. Biosci. 5, 1-18 (2018).
2. Wei Xu1, 2, 12, Hui Yang1, 2, 12, Ying Liu3, 12, Ying Yang1, Ping Wang1, Se-Hee Kim8, S., Ito8, 10, Chen Yang6, Pu Wang1, 2, Meng-Tao Xiao1, 2, Li-xia Liu5, Wen-qing Jiang1, 2, J., Liu6, Jin-ye Zhang2, Bin Wang4, Stephen Frye9, Yi Zhang8, 10, 11, Yan-hui Xu1, Q. & Lei2, 5, Kun-Liang Guan1, 2, 5, 7,*, Shi-min Zhao1, 2,*, and Yue Xiong1, 2, 8, 11. Control of Embryonic Stem Cell State Richard. Cancer Cell 29, 997-1003 (2012).
3. Kalliri, E., Mulrooney, S. B. & Hausinger, R. P. Identification of Escherichia coli YgaF as an L-2-hydroxyglutarate oxidase. J. Bacteriol. 190, 3793-3798 (2008).
4. Zhang, W. et al. Coupling between D-3-phosphoglycerate dehydrogenase and D-2-hydroxyglutarate dehydrogenase drives bacterial L-serine synthesis. Proc. Natl. Acad. Sci. U.S.A. 114, E7574-E7582 (2017).
5. Zhao, G. & Winkler, M. E. A novel alpha-ketoglutarate reductase activity of the serA-encoded 3-phosphoglycerate dehydrogenase of Escherichia coli K-12 and its possible implications for human 2-hydroxyglutaric aciduria. J. Bacteriol. 178, 232-9 (1996).
6. Zhang, X., Xu, G., Shi, J., Koffas, M. A. G. & Xu, Z. Microbial Production of L-Serine from Renewable Feedstocks. Trends Biotechnol. 36, 700-712 (2018).
7. Rennig, M. et al. Industrializing a Bacterial Strain for I-Serine Production through Translation Initiation Optimization. ACS Synth. Biol. 8, 2347-2358 (2019).
8. Geueke, B., Riebel, B. & Hummel, W. NADH oxidase from Lactobacillus brevis: A new catalyst for the regeneration of NAD. Enzyme Microb. Technol. 32, 205-211 (2003).
9. Marx, A., Eikmanns, B. J., Sahm, H., De Graaf, A. A. & Eggeling, L. Response of the Central Metabolism inCorynebacterium glutamicumto the use of an NADH-Dependent Glutamate Dehydrogenase. Metab. Eng. 1, 35-48 (1999).
10. Martínez, I., Zhu, J., Lin, H., Bennett, G. N. & San, K. Y. Replacing Escherichia coli NAD-dependent glyceraldehyde 3-phosphate dehydrogenase (GAPDH) with a NADP-dependent enzyme from Clostridium acetobutylicum facilitates NADPH dependent pathways. Metab. Eng. 10, 352-359 (2008).
11. King, Z. A. & Feist, A. M. Optimal cofactor swapping can increase the theoretical yield for chemical production in Escherichia coli and Saccharomyces cerevisiae. Metab. Eng. 24, 117-128 (2014).
12. Niu, H. et al. Metabolic engineering for improving l-tryptophan production in Escherichia coli. J. Ind. Microbiol. Biotechnol. 46, 55-65 (2019).
13. Hashim, Y., Ismail, N., Jamal, P., Othman, R. & Salleh, H. Production of Cysteine: Approaches, Challenges and Potential Solution. Int. J. Biotechnol. Wellness Ind. 3, 95-101 (2014).
14. Zhao, G. & Winkler, M. E. A novel α-ketoglutarate reductase activity of the sera-encoded 3-phosphoglycerate dehydrogenase of Escherichia coli K-12 and its possible implications for human 2-hydroxyglutaric aciduria. J. Bacteriol. 178, 232-239 (1996).
15. Mundhada, H. et al. Increased production of L-serine in Escherichia coli through Adaptive Laboratory Evolution. Metab. Eng. 39, 141-150 (2017).
16. Mundhada, H., Schneider, K., Christensen, H. B. & Nielsen, A. T. Engineering of high yield production of L-serine in Escherichia coli. Biotechnol. Bioeng. 113, 807-816 (2016).
17. Wang, Y., San, K. Y. & Bennett, G. N. Improvement of NADPH bioavailability in Escherichia coli by replacing NAD+-dependent glyceraldehyde-3-phosphate dehydrogenase GapA with NADP+-dependent GapB from Bacillus subtilis and addition of NAD kinase. J. Ind. Microbiol. Biotechnol. 40, 1449-1460 (2013).
18. Al-rabiee, R., Zhang, Y. & Grant, G. A. The Mechanism of Velocity Modulated Allosteric Regulation in D-3-Phosphoglycerate Dehydrogenase. 271, 23235-23238 (1996).
19. Ben Chorin A., Masrati G., Kessel A., Narunsky A., Sprinzak J., Lahav S., Ashkenazy H. and Ben-Tal N. (2020). ConSurf-DB: An accessible repository for the evolutionary conservation patterns of the majority of PDB proteins. Protein Science 29:258-267.
20. Goldenberg O., Erez E., Nimrod G. and Ben-Tal N. (2009). The ConSurf-DB: Pre-calculated evolutionary conservation profiles of protein structures. Nucleic Acids Research (Database issue), 37: D323-D327; PMID: 18971256.

### SEQUENCE LISTINGS

The present application contains a listing of sequences included in the below tables A and B submitted electronically in ST26 format which is hereby incorporated by reference in its entirety.

TABLE A
SEQ ID NO: 1 −> Protein sequence of HGA producing SerA from Escherichia coli
SEQ ID NO: 2 −> Protein sequence of HGA producing SerA from Haemophilusinfluenzae
SEQ ID NO: 3 −> Protein sequence of HGA producing SerA from Saccharomyces cerevisiae
SEQ ID NO: 4 −> Protein sequence of HGA producing SerA from Schizosaccharomycespombe
SEQ ID NO: 5 −> Protein sequence of HGA producing SerA from Neurosporacrassa
SEQ ID NO: 6 −> Protein sequence of non- HGA producing SerA from Entamoebahistolytica
SEQ ID NO: 7 −> Protein sequence of non- HGA producing SerA from Homo sapiens
SEQ ID NO: 8 −> Protein sequence of non- HGA producing SerA from Rattus norvegicus
SEQ ID NO: 9 −> Protein sequence of non- HGA producing SerA from Mus musculus
SEQ ID NO: 10 −> Protein sequence of non- HGA producing SerA from Mycobacterium tuberculosis
SEQ ID NO: 11 −> Protein sequence of non- HGA producing SerA from Bacillus subtilis
SEQ ID NO: 12 −> Protein sequence of non- HGA producing SerA from Methanobacterium sp.
SEQ ID NO: 13 −> Protein sequence of non- HGA producing SerA from Arabidopsis thaliana
SEQ ID NO: 14 −> Protein sequence of non- HGA producing SerA from Corynebacterium glutamicum
SEQ ID NO: 15 −> Protein sequence of non- HGA producing SerA from Streptomyces sp.
SEQ ID NO: 16 −> Protein sequence of feedback insensitive SerA from Escherichia coli
SEQ ID NO: 17 −> Protein sequence of feedback insensitive SerA from Escherichia coli
SEQ ID NO: 18 −> Protein sequence of feedback insensitive SerA from Escherichia coli
SEQ ID NO: 19 −> Protein sequence of feedback insensitive SerA from Corynebacterium glutamicum
SEQ ID NO: 20 −> Protein sequence of feedback insensitive SerA from Corynebacterium glutamicum
SEQ ID NO: 21 −> Protein sequence of feedback insensitive SerA from Corynebacterium glutamicum
SEQ ID NO: 22 −> Protein sequence of feedback insensitive SerA from Mycobacteriumtuberculosis
SEQ ID NO: 23 −> Protein sequence of NADH dependent Glyceraldehyde-3-phosphate dehydrogenase
from Clostridiumacetobutylicum
SEQ ID NO: 24 −> Protein sequence of NADH dependent Glyceraldehyde-3-phosphate dehydrogenase
from Bacillus subtilis
SEQ ID NO: 25 −> Protein sequence of NADH dependent Glyceraldehyde-3-phosphate dehydrogenase
from Corynebacterium glutamicum
SEQ ID NO: 26 −> Protein sequence of NADH dependent Glyceraldehyde-3-phosphate dehydrogenase
from Arabidopsis thaliana
SEQ ID NO: 27 −> Protein sequence of NADH dependent Glyceraldehyde-3-phosphate dehydrogenase
from Chlamydomonas reinhardtii
SEQ ID NO: 28 −> Protein sequence of NADH dependent Glyceraldehyde-3-phosphate dehydrogenase
from Paracoccus yeei
SEQ ID NO: 29 −> Protein sequence of NADH dependent Glyceraldehyde-3-phosphate dehydrogenase
from Celeribacter marinus
SEQ ID NO: 30 −> Protein sequence of NADH dependent Glyceraldehyde-3-phosphate dehydrogenase
from Bradyrhizobiaceae bacterium
SEQ ID NO: 31 −> Protein sequence of NADH dependent Glyceraldehyde-3-phosphate dehydrogenase
from Pseudomonas furukawaii
SEQ ID NO: 32 −> Protein sequence of NADH dependent Glyceraldehyde-3-phosphate dehydrogenase
from Bigelowiel lanatans
SEQ ID NO: 33 −> Protein sequence of NADH dependent Glyceraldehyde-3-phosphate dehydrogenase
from Pseudomonas chlororaphis
SEQ ID NO: 34 −> Protein sequence of NADH dependent Glyceraldehyde-3-phosphate dehydrogenase
from Saccharolobus solfataricus
SEQ ID NO: 35 −> Protein sequence of NADH dependent Glyceraldehyde-3-phosphate dehydrogenase
from Pseudomonas viridiflava
SEQ ID NO: 36 −> Protein sequence of NADH dependent Glyceraldehyde-3-phosphate dehydrogenase
from Kluyveromyces lactis
SEQ ID NO: 37 −> Protein sequence of NADH dependent Glyceraldehyde-3-phosphate dehydrogenase
from Geobacillus stearothermophilus
SEQ ID NO: 38 −> Protein sequence of NADPH dependent Glyceraldehyde-3-phosphate dehydrogenase
from Streptococcus pyogenes
SEQ ID NO: 39 −> Protein sequence of NADPH dependent Glyceraldehyde-3-phosphate dehydrogenase
from Thermoproteus tenax
SEQ ID NO: 40 −> Protein sequence of NADPH dependent Glyceraldehyde-3-phosphate dehydrogenase
from Streptococcus mutans
SEQ ID NO: 41 −> Protein sequence of NADPH dependent Glyceraldehyde-3-phosphate dehydrogenase
from Alkalihalobacillus halodurans
SEQ ID NO: 42 −> Protein sequence of NADPH dependent Glyceraldehyde-3-phosphate dehydrogenase
from Saccharolobus solfataricus
SEQ ID NO: 43 −> Protein sequence of NADPH dependent Glyceraldehyde-3-phosphate dehydrogenase
from Clostridium acetobutylicum
SEQ ID NO: 44 −> Protein sequence of NADPH dependent Glyceraldehyde-3-phosphate dehydrogenase
from Bacillus cereus
SEQ ID NO: 45 −> Protein sequence of NADPH dependent Glyceraldehyde-3-phosphate dehydrogenase
from Saccharolobus solfataricus
SEQ ID NO: 46 −> Protein sequence of NADPH dependent Glyceraldehyde-3-phosphate dehydrogenase
from Thermococcus sibiricus
SEQ ID NO: 47 −> Protein sequence of zwf - Glucose-6-phosphate 1-dehydrogenase from Escherichia coli
SEQ ID NO: 48 −> Protein sequence of pgl - 6-phosphogluconatedehydrogenase, decarboxylating
from Escherichia coli
SEQ ID NO: 49 −> Protein sequence of Nox - NADH oxidase from Lactobacillus brevis
SEQ ID NO: 50 −> Protein sequence of Nox - NADH oxidase from Levilactobacillus brevis
SEQ ID NO: 51 −> Protein sequence of Nox - NADH oxidase from Secundilactobacillus silagincola
SEQ ID NO: 52 −> Protein sequence of Nox - SidA/lucD/PvdA family monooxygenase from
Secundilactobacillus folii
SEQ ID NO: 53 −> Protein sequence of Nox - NADH oxidase from Lactobacilluskimchicus
SEQ ID NO: 54 −> Protein sequence of Nox - NADH peroxidase Npx from Lentilactobacillus curieae
SEQ ID NO: 55 −> Protein sequence of Nox - NADH oxidase from Lentilactobacillus parakefiri
SEQ ID NO: 56 −> Protein sequence of Nox - NADH oxidase from Lactobacillus farraginis
SEQ ID NO: 57 −> Protein sequence of GDH - NAD-specific glutamate dehydrogenase(NAD-GDH)
from Staphylococcus aureus
SEQ ID NO: 58 −> Protein sequence of GDH - Catabolic NAD-specific glutamate dehydrogenase
RocG (NAD-GDH) from Bacillus subtilis
SEQ ID NO: 59 −> Protein sequence of GDH - NAD-specific glutamate dehydrogenase (NAD-GDH)
from Pseudomonas aeruginosa
SEQ ID NO: 60 −> Protein sequence of GDH - NAD-specific glutamate dehydrogenase(NAD-GDH)
from Clostridium symbiosum
SEQ ID NO: 61 −> Protein sequence of GDH - NAD-specific glutamate dehydrogenase(NAD-GDH)
from Saccharomyces cerevisiae
SEQ ID NO: 62 −> Protein sequence of GDH - (GDH) from Pyrococcus furiosus
SEQ ID NO: 63 −> Protein sequence of GDH - Glutamate dehydrogenase(GDH) from Thermococcus
kodakarensis
SEQ ID NO: 64 −> Protein sequence of GDH - Glutamate dehydrogenase from Lactobacillus sp.
SEQ ID NO: 65 −> Protein sequence of GDH - Glutamate dehydrogenase from Streptomyces sp.
SEQ ID NO: 66 −> Protein sequence of GDH - Glutamate dehydrogenase from Brevibacterium sp.
SEQ ID NO: 67 −> DNA sequence of Primer serA-Ncol
SEQ ID NO: 68 −> DNA sequence of Primer serA Notl_R
SEQ ID NO: 69 −> DNA sequence of Primer serB_Ncol_F
SEQ ID NO: 70 −> DNA sequence of Primer serB_R_HindIII
SEQ ID NO: 71 −> DNA sequence of Primer serB_Pacl_R
SEQ ID NO: 72 −> DNA sequence of Primer serC_F_Ndel
SEQ ID NO: 73 −> DNA sequence of Primer serC_R_Pacl
SEQ ID NO: 74 −> DNA sequence of Primer ydeD_F_Ndel
SEQ ID NO: 75 −> DNA sequence of Primer ydeD_cHis_R-Pacl
SEQ ID NO: 76 −> DNA sequence of Primer ydeD_Ncol_F1
SEQ ID NO: 77 −> DNA sequence of Primer serA N364A_F
SEQ ID NO: 78 −> DNA sequence of Primer serA N364A_R
SEQ ID NO: 79 −> DNA sequence of Primer serA H344AN346A_F
SEQ ID NO: 80 −> DNA sequence of Primer serA H344AN346A_R
SEQ ID NO: 81 −> DNA sequence of Primer serC_Ncolc_F
SEQ ID NO: 82 −> DNA sequence of Primer serC_Ncolc_R
SEQ ID NO: 83 −> DNA sequence of Primer gapC_camsacB_F
SEQ ID NO: 84 −> DNA sequence of Primer gapC_camsacB_R
SEQ ID NO: 85 −> DNA sequence of Primer gapC_aF
SEQ ID NO: 86 −> DNA sequence of Primer gapC_aR
SEQ ID NO: 87 −> DNA sequence of Primer pSEVA27_f wd
SEQ ID NO: 88 −> DNA sequence of Primer pSEVA27_T7_rev
SEQ ID NO: 89 −> DNA sequence of Primer SerA-C_rev
SEQ ID NO: 90 −> DNA sequence of Primer SerA-C_fwd
SEQ ID NO: 91 −> DNA sequence of Primer SerC-B_rev
SEQ ID NO: 92 −> DNA sequence of Primer SerC-B_fwd
SEQ ID NO: 93 −> DNA sequence of Primer serB_UR
SEQ ID NO: 94 −> DNA sequence of Primer serB_Nox_UR
SEQ ID NO: 95 −> DNA sequence of Primer pSEVA_Nox_UF
SEQ ID NO: 96 −> DNA sequence of Primer nox_lb100_UF
SEQ ID NO: 97 −> DNA sequence of Primer Nox_lb_UR
SEQ ID NO: 98 −> DNA sequence of Primer rocG_UR
SEQ ID NO: 99 −> DNA sequence of Primer rocG_UF
SEQ ID NO: 100 −> DNA sequence of Primer pACYC_UF
SEQ ID NO: 101 −> DNA sequence of Primer pACYC_UR
SEQ ID NO: 102 −> DNA sequence of Primer pCDF_VB_UF
SEQ ID NO: 103 −> DNA sequence of Primer pCDF_UR
SEQ ID NO: 104 −> DNA sequence of Primer serAglut_pCDF_UF
SEQ ID NO: 105 −> DNA sequence of Primer serAglut_pCDF_UR
SEQ ID NO: 106 −> DNA sequence of Primer pSEVA_UR
SEQ ID NO: 107 −> DNA sequence of Primer pSEVA_UF
SEQ ID NO: 108 −> DNA sequence of Primer Cglut_pSEVA_UF
SEQ ID NO: 109 −> DNA sequence of Primer Cglut_pSEVA_UR
SEQ ID NO: 110 −> DNA sequence of Primer CglutserA N483A_F
SEQ ID NO: 111 −> DNA sequence of Primer CglutserA N483A_R
SEQ ID NO: 112 −> DNA sequence of Primer CglutserA Y463A_F
SEQ ID NO: 113 −> DNA sequence of Primer CglutserA Y463A_R
SEQ ID NO: 114 −> DNA sequence encoding Nox - NADH oxidase from Lactobacillus brevis
SEQ ID NO: 115 −> DNA sequence encoding Nox - SidA/lucD/PvdA family monooxygenase from
Secundilactobacillus folii
SEQ ID NO: 116 −> DNA sequence encoding Nox - NADH oxidase from Lentilactobacillus parakefiri
SEQ ID NO: 117 −> Protein sequence of SerC - phosphoserine aminotransferase (PSAT) from
Escherichia coli
SEQ ID NO: 118 −> Protein sequence of SerB - phosphoserine phosphatase (PSPH) from Escherichia coli
SEQ ID NO: 119 −> Protein sequence of Pgk - Phosphoglycerate kinase from Escherichia coli
SEQ ID NO: 120 −> DNA sequence of Primer serA_rat_UF
SEQ ID NO: 121 −> DNA sequence of Primer serA_rat_UR
SEQ ID NO: 122 −> DNA sequence of Primer serA_rat_UF_pBB
SEQ ID NO: 123 −> DNA sequence of Primer serA_rat_UR_pBB
SEQ ID NO: 124 −> DNA sequence of Primer trunc-serA_rat_Uf
SEQ ID NO: 125 −> DNA sequence of Primer trunc-serA_rat_UR
SEQ ID NO: 126 −> DNA sequence of Primer trunc-serA_rat_UF_pBB
SEQ ID NO: 127 −> DNA sequence of Primer trunc-serA_rat_UR_pBB
SEQ ID NO: 128 −> DNA sequence of Primer gap_Cglit_F
SEQ ID NO: 129 −> DNA sequence of Primer gap_Cglut_R
SEQ ID NO: 130 −> DNA sequence of Primer gap_Klactis_F
SEQ ID NO: 131 −> DNA sequence of Primer gap_Klactis_R
SEQ ID NO: 132 −> DNA sequence of Primer nox_UF
SEQ ID NO: 133 −> DNA sequence of Primer nox_UR
SEQ ID NO: 134 −> DNA sequence of Primer nox_UF_pBB
SEQ ID NO: 135 −> DNA sequence of Primer nox_UR_pBB
SEQ ID NO: 136 −> DNA sequence of Primer zwf_UF
SEQ ID NO: 137 −> DNA sequence of Primer zwf_UR
SEQ ID NO: 138 −> DNA sequence of Primer zwf_UF_pBB
SEQ ID NO: 139 −> DNA sequence of Primer zwf_UR_pBB
SEQ ID NO: 140 −> DNA sequence of Primer pgl_UF
SEQ ID NO: 141 −> DNA sequence of Primer pgl_UR
SEQ ID NO: 142 −> DNA sequence of Primer pgl_UF_pBB
SEQ ID NO: 143 −> DNA sequence of Primer pgl_UR_pBB
SEQ ID NO: 144 −> DNA sequence encoding truncated rat SerA

TABLE 1
Proteins used in the present invention.

| Seq | UNIPROT ID | Enzyme name | Sequence |
|---|---|---|---|
| 1 | D-3- | Escherichia coli | MAKVSLEKDKIKFLLVEGVHQKALESLRAAGYTNIEFH |
| | phosphoglycerate | (strain K12) | KGALDDEQLKESIRDAHFIGLRSRTHLTEDVINAAEKLV |
| | dehydrogenase | | AIGCFCIGTNQVDLDAAAKRGIPVFNAPFSNTRSVAEL |
| | (PGDH) | | VIGELLLLLRGVPEANAKAHRGVWNKLAAGSFEARGK |
| | | | KLGIIGYGHIGTQLGILAESLGMYVYFYDIENKLPLGNA |
| | | | TQVQHLSDLLNMSDVVSLHVPENPSTKNMMGAKEIS |
| | | | LMKPGSLLINASRGTVVDIPALCDALASKHLAGAAIDV |
| | | | FPTEPATNSDPFTSPLCEFDNVLLTPHIGGSTQEAQENI |
| | | | GLEVAGKLIKYSDNGSTLSAVNFPEVSLPLHGGRRLMH |
| | | | IHENRPGVLTALNKIFAEQGVNIAAQYLQTSAQMGYV |
| | | | VIDIEADEDVAEKALQAMKAIPGTIRARLLY |
| 2 | 2-oxoglutarate | Haemophilus | MTNKVSLDKSKIKFVLFEGVHQSALDTLHSAGYSNIDY |
| | reductase (EC | influenzae | YKKALDGDELKEAIKDAHFIGLRSRTHLTAEMIEAAPKL |
| | 1.1.1.399) (EC | | IAVGCFCIGTNQVDLNAAKARGIPVFNAPFSNTRSVAE |
| | 1.1.1.95) (D-3- | | LVLGEILLLMRNVPQANAEVHRGVWNKSATGSYEVR |
| | phosphoglycerate | | GKKLGIIGYGHIGSQLSIIAESLGMDVYFYDIENKLPLGN |
| | dehydrogenase) | | AKQVRSLEELLSSCDVISLHVPELPSTKNLMNATRIAQL |
| | | | KQGAILINAARGTIVDIDALAQALKDGKIHGAAIDVFPV |
| | | | EPASINEEFVSPLREFDNVILTPHIGGSTAEAQENIGFEV |
| | | | AGKFVKYSDNGSTLSSVNFPEVSLPEHEGTKRLLHIHE |
| | | | NRPGILNKLNQIFSEAHLNIAAQYLQTDPKIGYVVVDV |
| | | | ETDDTSLLLTKLKEVDGTIRARVLY |
| 3 | D-3- | Saccharomyces | MSYSAADNLQDSFQRAMNFSGSPGAVSTSPTQSFM |
| | phosphoglycerate | cerevisiae | NTLPRRVSITKQPKALKPFSTGDMNILLLENVNATAIKI |
| | dehydrogenase | (strain ATCC | FKDQGYQVEFHKSSLPEDELIEKIKDVHAIGIRSKTRLTE |
| | 2 (3-PGDH 2)(EC | 204508/S288c) | KILQHARNLVCIGCFCIGTNQVDLKYAASKGIAVFNSPF |
| | 1.1.1.95) (2- | (Baker's yeast) | SNSRSVAELVIGEIISLARQLGDRSIELHTGTWNKVAAR |
| | oxoglutarate | | CWEVRGKTLGIIGYGHIGSQLSVLAEAMGLHVLYYDIV |
| | reductase) (EC | | TIMALGTARQVSTLDELLNKSDFVTLHVPATPETEKML |
| | 1.1.1.399) | | SAPQFAAMKDGAYVINASRGTVVDIPSLIQAVKANKIA |
| | | | GAALDVYPHEPAKNGEGSFNDELNSWTSELVSLPNIIL |
| | | | TPHIGGSTEEAQSSIGIEVATALSKYINEGNSVGSVNFP |
| | | | EVSLKSLDYDQENTVRVLYIHRNVPGVLKTVNDILSDH |
| | | | NIEKQFSDSHGEIAYLMADISSVNQSEIKDIYEKLNQTS |
| | | | AKVSIRLLY |
| 4 | Putative D-3- | Schizosaccharo- | MDIKGGRRGNVEDSLNKLSLSPPDNNSSFLSNHFQVR |
| | phosphoglycerate | myces pombe | KSYSQAPARTLKPFASEDIKILLLENVNQSALSNLKDEG |
| | dehydrogenase | (strain 972/ | YQVEFLKTSMSEDDLVEKIKGVHAIGIRSKTRLTRRVLE |
| | (3-PGDH) (EC | ATCC 24843) | AADSLIVIGCFCIGTNQVDLDFAAERGIAVFNSPYANSR |
| | 1.1.1.95) (2- | (Fission yeast) | SVAELVIGYIISLARQVGDRSLELHRGEWNKVSSGCWE |
| | oxoglutarate | | IRGKTLGIIGYGHIGSQLSVLAEAMGLHVVYYDILPIMP |
| | reductase) (EC | | LGSAKQLSSLPELLHRADFVSLHVPASPETKNMISSKEF |
| | 1.1.1.399) | | AAMKEGSYLINASRGTVVDIPALVDASKSGKIAGAAID |
| | | | VYPSEPAGNGKDKFVDSLNSWTSELTHCKNIILTPHIG |
| | | | GSTEEAQYNIGIEVSEALTRYINEGNSIGAVNFPEVSLR |
| | | | SLTEADRNAARVLFVHRNVPGVLRQVNELFIDHNIKS |
| | | | QFSDSRGDIAYLVADISDCTPGSLEALHQKLESLPCKIN |
| | | | TRLLY |
| 5 | Phosphoglycerate | Neurospora | MTVPREIAQPTGDKTAAQFSVSPSTFQSPISSSSFMAS |
| | dehydrogenase | crassa (strain | ALAHRSAPKQLKPFNTEDVKILLLENVNQTGQDILRAQ |
| | (EC 1.1.1.95) | ATCC 24698/ | GYQVEFLKTSLPEDQLIEKIKDVHVIGIRSKTKLTANVLR |
| | | 74-OR23-1A/ | EAKNLLVIGCFCIGTNQVDLDYAAQHGIAVFNSPFANS |
| | | CBS 708.71/ | RSVAELVIAEIITLARQLGDRSNEMHRGTWNKVSAKC |
| | | DSM 1257/ | WEIRGKTLGIVGYGHIGSQLSVLAEAMGMTVIYYDVV |
| | | FGSC 987) | TLMAIGTARQVPTLEDLLAEADFVTLHVPETPETKNMI |
| | | | SNDQFEKMKTGSYLINASRGTVVDIPALIKASRSGKIAG |
| | | | AALDVFPNEPAANGDYFTNDLNQWGEDLRGLNNIILT |
| | | | PHIGGSTEEAQRAIGVEVADALVRYINQGVTTGSVNV |
| | | | PEVTMRSLTLDEPNHARVIYIHRNVPGVLRKVNEILAE |
| | | | HNVDKQISDSKGDLAYLMADVSDVKQDDIKNIRDSLD |
| | | | SLSSRILTRILY |
| 6 | Phosphoglycerate | Entamoeba | MKIVVITEKPFAENAVKGIREILEKAGHEVVMIEKYKKK |
| | dehydrogenase | histolytica | EDVIERIKDADGVIVRSDKIDEEIIKAGEKVKIIVRAGAG |
| | | | YDNIDIEACNQGKIVVMNTPGQNRNGVAELCIGMMI |
| | | | FGFRKGFKEGKGRELKDKTLGICGCGYVGKRVKEIAEGI |
| | | | GMKIKVYDPFITTENQVKKIEELFEECQVISLHLPLTKET |
| | | | KGKIGYELIKKLPYGGMICNTARKEIIDEEGLIRIMRERE |
| | | | DLIYITDVAPTSKVFNNEFKGRFFATPIKIGAETEESNIN |
| | | | AGMAAASQICDFFTNGTVKFQVNKF |
| 7 | Phosphoglycerate | Homo sapiens | MAFANLRKVLISDSLDPCCRKILQEGGLQVVEKQNLSK |
| | dehydrogenase | (Human) | EELIAELQDCEGLIVRSATKVTADVINAAEKLQVVGRA |
| | | | GTGVDNVDLEAATRKGILVMNTPNGNSLSAAELTCG |
| | | | MIMCLARQIPQATASMKDGKWERKKFMGTELNGKT |
| | | | LGILGLGRIGREVATRMQSFGMKTIGYDPIISPEVSASF |
| | | | GVQQLPLEEIWPLCDFITVHTPLLPSTTGLLNDNTFAQ |
| | | | CKKGVRVVNCARGGIVDEGALLRALQSGQCAGAALD |
| | | | VFTEEPPRDRALVDHENVISCPHLGASTKEAQSRCGEE |
| | | | IAVQFVDMVKGKSLTGVVNAQALTSAFSPHTKPWIGL |
| | | | AEALGTLMRAWAGSPKGTIQVITQGTSLKNAGNCLSP |
| | | | AVIVGLLKEASKQADVNLVNAKLLVKEAGLNVTTSHSP |
| | | | AAPGEQGFGECLLAVALAGAPYQAVGLVQGTTPVLQ |
| | | | GLNGAVFRPEVPLRRDLPLLLFRTQTSDPAMLPTMIGL |
| | | | LAEAGVRLLSYQTSLVSDGETWHVMGISSLLPSLEAW |
| | | | KQHVTEAFQFHF |
| 8 | Phosphoglycerate | Rattus | MAFANLRKILISDSLDPCCRKILQDGGLQVVEKQNLSK |
| | dehydrogenase | norvegicus (Rat) | EELIAELQDCEGLIVRSATKVTADVINAAEKLQVVGRA |
| | | | GTGVDNVDLEAATRKGVLVMNTPNGNSLSAAELTCG |
| | | | MLMCLARQIPQATASMKDGKWDRKKFMGTELNGKT |
| | | | LGILGLGRIGREVAARMQAFGMKTVGYDPIISPEVAAS |
| | | | FGVQQLPLEEIWPLCDFITVHTPLLPSTTGLLNDSTFAQ |
| | | | CKKGVRVVNCARGGIVDEGALLRALQSGQCAGAALD |
| | | | VFTEEPPRDRALVDHENVISCPHLGASTKEAQSRCGEE |
| | | | IAVQFVDMVKGKSLTGVVNAQALTSAFSPHTKPWIGL |
| | | | AEALGTLMHAWAGSPKGTIQVVTQGTSLKNAGTCLS |
| | | | PAVIVGLLREASKQADVNLVNAKLLVKEAGLNVTTSHS |
| | | | PGVPGEQGIGECLLTVALAGAPYQAVGLVQGTTPML |
| | | | QMLNGAVFRPEVPLRRGQPLLLFRAQPSDPVMLPTM |
| | | | IGLLAEAGVQLLSYQTSKVSDGDTWHVMGLSSLLPSL |
| | | | DAWKQHVSEAFQFCF |
| 9 | Phosphoglycerate | Mus musculus | MAFANLRKVLISDSLDPCCRKILQDGGLQVVEKQNLSK |
| | dehydrogenase | (Mouse) | EELIAELQDCEGLIVRSATKVTADVINAAEKLQVVGRA |
| | | | GTGVDNVDLEAATRKGILVMNTPNGNSLSAAELTCG |
| | | | MIMCLARQIPQATASMKDGKWDRKKFMGTELNGKT |
| | | | LGILGLGRIGREVATRMQSFGMKTVGYDPIISPEVAAS |
| | | | FGVQQLPLEEIWPLCDFITVHTPLLPSTTGLLNDSTFAQ |
| | | | CKKGVRVVNCARGGIVDEGALLRALQSGQCAGAALD |
| | | | VFTEEPPRDRALVDHENVISCPHLGASTKEAQSRCGEE |
| | | | IAVQFVDMVKGKSLTGVVNAQALTSAFSPHTKPWIGL |
| | | | AEAMGTLMHAWAGSPKGTIQVVTQGTSLKNAGTCL |
| | | | SPAVIVGLLREASKQADVNLVNAKLLVKEAGLNVTTSH |
| | | | NPGVPGEQGSGECLLTVALAGAPYQAVGLVQGTTPM |
| | | | LQMLNGAVFRPEVPLRRGQPLLVFRAQPSDPGMLPT |
| | | | MIGLLAEAGVQLLSYQTSMVSDGEPWHVMGLSSLLP |
| | | | SLETWKQHVLEAFQFCF |
| 10 | Phosphoglycerate | Mycobacterium | MSLPVVLIADKLAPSTVAALGDQVEVRWVDGPDRDK |
| | dehydrogenase | tuberculosis | LLAAVPEADALLVRSATTVDAEVLAAAPKLKIVARAGV |
| | | | GLDNVDVDAATARGVLVVNAPTSNIHSAAEHALALLL |
| | | | AASRQIPAADASLREHTWKRSSFSGTEIFGKTVGVVGL |
| | | | GRIGQLVAQRIAAFGAYVVAYDPYVSPARAAQLGIELL |
| | | | SLDDLLARADFISVHLPKTPETAGLIDKEALAKTKPGVII |
| | | | VNAARGGLVDEAALADAITGGHVRAAGLDVFATEPC |
| | | | TDSPLFELAQVVVTPHLGASTAEAQDRAGTDVAESVR |
| | | | LALAGEFVPDAVNVGGGVVNEEVAPWLDLVRKLGVL |
| | | | AGVLSDELPVSLSVQVRGELAAEEVEVLRLSALRGLFSA |
| | | | VIEDAVTFVNAPALAAERGVTAEICKASESPNHRSVVD |
| | | | VRAVGADGSVVTVSGTLYGPQLSQKIVQINGRHFDLR |
| | | | AQGINLIIHYVDRPGALGKIGTLLGTAGVNIQAAQLSE |
| | | | DAEGPGATILLRLDQDVPDDVRTAIAAAVDAYKLEVV |
| | | | DLS |
| 11 | Phosphoglycerate | Bacillus subtilis | MFRVLVSDKMSNDGLQPLIESDFIEIVQKNVADAEDEL |
| | dehydrogenase | | HTFDALLVRSATKVTEDLFNKMTSLKIVGRAGVGVDNI |
| | | | DIDEATKHGVIVINAPNGNTISTAEHTFAMISSLMRHIP |
| | | | QANISVKSREWNRTAYVGSELYGKTLGIVGLGRIGSEIA |
| | | | QRARAFGMTVHVFDPFLTEERAKKIGVNSRTFEEVLES |
| | | | ADIITVHTPLTKETKGLLNKETIAKTKKGVRLINCARGGII |
| | | | DEAALLEALENGHVAGAALDVFEVEPPVDNKLVDHPL |
| | | | VIATPHLGASTKEAQLNVAAQVSEEVLQFAKGLPVMS |
| | | | AINLPAMTKDEFAKIKPYHQIAGKIGSLVSQCMKEPVQ |
| | | | DVAIQYEGTIAKLETSFITKALLSGFLKPRVDSTVNEVN |
| | | | AGGVAKERGISFSEKISSSESGYDNCISVKVTGDRSTFT |
| | | | VTATYIPHFGERIVEINGFNIDFYPTGHLVYIQHQDTTG |
| | | | VIGRVGRILGDNDINIATMQVGRKEKGGEAIMMLSFD |
| | | | RHLEDKIVKELTNVPDIVSVKLIDLP |
| 12 | Phosphoglycerate | Methanobacterium | MKVIIADSINEKGISDLKEVAEVTVATSITPEELVEVIKEY |
| | dehydrogenase | sp. | DAIVVRSRTKVTREVIEAGSKLKVIARAGVGVDNVDVQ |
| | | | AATEKGIMVINAPESTSITVAEHSMGLMMSLARKISIA |
| | | | DKSVKSGQWEKSKFMGIELNNKTLGIVGMGRIGSQV |
| | | | VIRAKAFNMEILVYDPYISKEAGAEMGVSVVDLETLLK |
| | | | ESDVITIHVPLTPETRHLISTREMEIMKNTAFIINCARGG |
| | | | IVDEDALYYALKNDIIGGAALDVFESEPPQESPLLELDN |
| | | | LIATPHIGASTAEAQRDAALIVANEIITVFQGGSPRNVL |
| | | | NMPVMDSKNFKIMKPYLDLSEKMGSFMEQATSGNIK |
| | | | NMDVTYCGELSEMPQQDILTRTMLQGILNPILTEPVN |
| | | | LVNAPSIAQNRGIIVTEGKRCESEGYKCIIKLEIKTDKDEF |
| | | | SLDGTYVDEPKIIKINNYWVDVKPEGTMVIAKYQDLPG |
| | | | TIGAIGTKLGEHEINIATMQVGRKEVGGEAIMVLKVD |
| | | | QSVPQSVIDEVKKMDHVEDAVALEF |
| 13 | Phosphoglycerate | Arabidopsis | MAFSSSCSSVKAVNSRWTSPSPSPSSRFAVLPAFLHRR |
| | dehydrogenase | thaliana | YATSVKLTAISAALKTVEQTTLTEDNRFSTVGSDSDEYN |
| | | (Mouse-ear | PTLPKPRILVTEKLGEAGVNLLREFGDVDCSYDLSPEDL |
| | | cress) | KKKVAESDALIVRSGTKVTREVFEAAKGRLKVVGRAGV |
| | | | GIDNVDLQAATEHGCLVVNAPTANTVAAAEHGIALLA |
| | | | SMARNVAQADASIKAGKWERSKYVGVSLVGKTLAV |
| | | | MGFGKVGTEVARRAKGLGMTVISHDPYAPADRARAL |
| | | | GVDLVSFDQAISTADFVSLHMPLTPATKKVFNDETFSK |
| | | | MKKGVRLINVARGGVIDEDALVRALDAGIVAQAALDV |
| | | | FCEEPPSKDSRLIQHENVTVTPHLGASTKEAQEGVAIEI |
| | | | AEAVAGALKGELSATAVNAPMVAPEVLSELTPYIVLAE |
| | | | KLGRLAVQLASGGKGVQSIRVVYRSARDRDDLDTRLL |
| | | | RAMITKGIIEPISDSYVNLVNADFIAKQKGLRISEERMV |
| | | | VDSSPEYPVDSIQVQILNVESNFAGAVSDAGDISIEGKV |
| | | | KYGVPHLTCVGSFGVDVSLEGNLILCRQVDQPGMIGQ |
| | | | VGNILGEQNVNVNFMSVGRTVLRKQAIMAIGVDEEP |
| | | | DNKTLERIGGVSAIEEFVFLKL |
| 14 | Phosphoglycerate | Corynebacterium | MSQNGRPVVLIADKLAQSTVDALGDAVEVRWVDGP |
| | dehydrogenase | glutamicum | NRPELLDAVKEADALLVRSATTVDAEVIAAAPNLKIVG |
| | | (strain R) | RAGVGLDNVDIPAATEAGVMVANAPTSNIHSACEHAI |
| | | | SLLLSTARQIPAADATLREGEWKRSSFNGVEIFGKTVGI |
| | | | VGFGHIGQLFAQRLAAFETTIVAYDPYANPARAAQLN |
| | | | VELVELDELMSRSDFVTIHLPKTKETAGMFDAQLLAKS |
| | | | KKGQIIINAARGGLVDEQALADAIESGHIRGAGFDVYS |
| | | | TEPCTDSPLFKLPQVVVTPHLGASTEEAQDRAGTDVA |
| | | | DSVLKALAGEFVADAVNVSGGRVGEEVAVWMDLAR |
| | | | KLGLLAGKLVDAAPVSIEVEARGELSSEQVDALGLSAV |
| | | | RGLFSGIIEESVTFVNAPRIAEERGLDISVKTNSESVTHR |
| | | | SVLQVKVITGSGASATVVGALTGLERVEKITRINGRGLD |
| | | | LRAEGLNLFLQYTDAPGALGTVGTKLGAAGIAIEAAAL |
| | | | TQAEKGDGAVLILRVESAVSEELEAEINAELGATSFQV |
| | | | DLD |
| 15 | Phosphoglycerate | Streptomyces | MSSKPVVLIAEELSPATVDALGPDFEIRYCNGADRAELL |
| | dehydrogenase | sp. F-3 | PAIADVDAILIRSATKVDAEAIAAANRLKVVARAGVGL |
| | | | DNVDVPAATKAGVMVVNAPTSNIVTAAELACGLILST |
| | | | ARNIPQANAALKSGEWKRSKYTGVELAEKTLGVVGLG |
| | | | RIGALVAQRMSAFGMRIVAYDPYVQPARAAQMGVR |
| | | | MLSLDELLQVSDFITVHLPKTPETIGLIGEEALRKVKPSV |
| | | | RIINAARGGIVDEEALYSALKEGRVAGAGLDVYAKEPC |
| | | | TDSPLFEFDQVICTPHLGASTDEAQEKAGIAVAKSVRL |
| | | | ALAGELVPDAVNVQGGVIAEDVKPGLPLAERLGRIFTA |
| | | | LAGEVAVRLDVEVYGEITQHDVKVLELSALKGVFEDVV |
| | | | DETVSYVNAPLFAQERGVEVRLTTSSEATDHRNVVTV |
| | | | RGTLANGEEVSVSGTLSGPKHVQKIVAVGEYDVDLAL |
| | | | ADHMIVLRYEDRPGVVGTVGRILGEAGINIAGMQVA |
| | | | RATAGGEALAVLTVDDTVSAAVLNELATEIGATSARSV |
| | | | NLV |
| 16 | D-3- | Escherichia coli | MAKVSLEKDKIKFLLVEGVHQKALESLRAAGYTNIEFH |
| | phosphoglycerate | (strain K12) | KGALDDEQLKESIRDAHFIGLRSRTHLTEDVINAAEKLV |
| | dehydrogenase | | AIGCFCIGTNQVDLDAAAKRGIPVFNAPFSNTRSVAEL |
| | (PGDH) | | VIGELLLLLRGVPEANAKAHRGVWNKLAAGSFEARGK |
| | | | KLGIIGYGHIGTQLGILAESLGMYVYFYDIENKLPLGNA |
| | | | TQVQHLSDLLNMSDVVSLHVPENPSTKNMMGAKEIS |
| | | | LMKPGSLLINASRGTVVDIPALCDALASKHLAGAAIDV |
| | | | FPTEPATNSDPFTSPLCEFDNVLLTPHIGGSTQEAQENI |
| | | | GLEVAGKLIKYSD** |
| 17 | D-3- | Escherichia coli | MAKVSLEKDKIKFLLVEGVHQKALESLRAAGYTNIEFH |
| | phosphoglycerate | (strain K12) | KGALDDEQLKESIRDAHFIGLRSRTHLTEDVINAAEKLV |
| | dehydrogenase | | AIGCFCIGTNQVDLDAAAKRGIPVFNAPFSNTRSVAEL |
| | (PGDH) | | VIGELLLLLRGVPEANAKAHRGVWNKLAAGSFEARGK |
| | | | KLGIIGYGHIGTQLGILAESLGMYVYFYDIENKLPLGNA |
| | | | TQVQHLSDLLNMSDVVSLHVPENPSTKNMMGAKEIS |
| | | | LMKPGSLLINASRGTVVDIPALCDALASKHLAGAAIDV |
| | | | FPTEPATNSDPFTSPLCEFDNVLLTPHIGGSTQEAQENI |
| | | | GLEVAGKLIKYSDNGSTLSAVNFPEVSLPLHGGRRLMH |
| | | | IAEARPGVLTALNKIFAEQGVAIAAQYLQTSAQMGYV |
| | | | VIDIEADEDVAEKALQAMKAIPGTIRARLLY* |
| 18 | D-3- | Escherichia coli | MAKVSLEKDKIKFLLVEGVHQKALESLRAAGYTNIEFH |
| | phosphoglycerate | (strain K12) | KGALDDEQLKESIRDAHFIGLRSRTHLTEDVINAAEKLV |
| | dehydrogenase | | AIGCFCIGTNQVDLDAAAKRGIPVFNAPFSNTRSVAEL |
| | (PGDH) | | VIGELLLLLRGVPEANAKAHRGVWNKLAARSFEARGK |
| | | | KLGIIGYGHIGTQLGILAESLGMYVYFYDIENKLPLGNA |
| | | | TQVQHLSDLLNMSDVVSLHVPENPSTKNMMGAKEIS |
| | | | LMKPGSLLINASRGTVVDIPALCDALASKHLAGAAIDV |
| | | | FPTEPATNSDPFTSPLCEFDNVLLTPHIGGSTQEAQENI |
| | | | GLEVAGKLIKYSD** |
| 19 | Phosphoglycerate | Corynebacterium | MSQNGRPVVLIADKLAQSTVDALGDAVEVRWVDGP |
| | dehydrogenase | glutamicum | NRPELLDAVKEADALLVRSATTVDAEVIAAAPNLKIVG |
| | | (strain R) | RAGVGLDNVDIPAATEAGVMVANAPTSNIHSACEHAI |
| | | | SLLLSTARQIPAADATLREGEWKRSSFNGVEIFGKTVGI |
| | | | VGFGHIGQLFAQRLAAFETTIVAYDPYANPARAAQLN |
| | | | VELVELDELMSRSDFVTIHLPKTKETAGMFDAQLLAKS |
| | | | KKGQIIINAARGGLVDEQALADAIESGHIRGAGFDVYS |
| | | | TEPCTDSPLFKLPQVVVTPHLGASTEEAQDRAGTDVA |
| | | | DSVLKALAGEFVADAVNVSGGRVGEEVAVWMDLAR |
| | | | KLG |
| 20 | Phosphoglycerate | Corynebacterium | MSQNGRPVVLIADKLAQSTVDALGDAVEVRWVDGP |
| | dehydrogenase | glutamicum | NRPELLDAVKEADALLVRSATTVDAEVIAAAPNLKIVG |
| | | (strain R) | RAGVGLDNVDIPAATEAGVMVANAPTSNIHSACEHAI |
| | | | SLLLSTARQIPAADATLREGEWKRSSFNGVEIFGKTVGI |
| | | | VGFGHIGQLFAQRLAAFETTIVAYDPYANPARAAQLN |
| | | | VELVELDELMSRSDFVTIHLPKTKETAGMFDAQLLAKS |
| | | | KKGQIIINAARGGLVDEQALADAIESGHIRGAGFDVYS |
| | | | TEPCTDSPLFKLPQVVVTPHLGGSTEEAQDRAGTDVA |
| | | | DSVLKALAGEFVADAVNVSGGRVGEEVAVWMDLAR |
| | | | KLGLLAGKLVDAAPVSIEVEARGELSSEQVDALGLSAV |
| | | | RGLFSGIIEESVTFVNAPRIAEERGLDISVKTNSESVTHR |
| | | | SVLQVKVITGSGASATVVGALTGLERVEKITRINGRGLD |
| | | | LRAEGLNLFLQATDAPGALGTVGTKLGAAGINIEAAAL |
| | | | TQAEKGDGAVLILRVESAVSEELEAEINAELGATSFQV |
| | | | DLD |
| 21 | Phosphoglycerate | Corynebacterium | MSQNGRPVVLIADKLAQSTVDALGDAVEVRWVDGP |
| | dehydrogenase | glutamicum | NRPELLDAVKEADALLVRSATTVDAEVIAAAPNLKIVG |
| | | (strain R) | RAGVGLDNVDIPAATEAGVMVANAPTSNIHSACEHAI |
| | | | SLLLSTARQIPAADATLREGEWKRSSFNGVEIFGKTVGI |
| | | | VGFGHIGQLFAQRLAAFETTIVAYDPYANPARAAQLN |
| | | | VELVELDELMSRSDFVTIHLPKTKETAGMFDAQLLAKS |
| | | | KKGQIIINAARGGLVDEQALADAIESGHIRGAGFDVYS |
| | | | TEPCTDSPLFKLPQVVVTPHLGASTEEAQDRAGTDVA |
| | | | DSVLKALAGEFVADAVNVSGGRVGEEVAVWMDLAR |
| | | | KLGLLAGKLVDAAPVSIEVEARGELSSEQVDALGLSAV |
| | | | RGLFSGIIEESVTFVNAPRIAEERGLDISVKTNSESVTHR |
| | | | SVLQVKVITGSGASATVVGALTGLERVEKITRINGRGLD |
| | | | LRAEGLNLFLQYTDAPGALGTVGTKLGAAGINIEAAAL |
| | | | TQAEKGDGAVLILRVESAVSEELEAEINAELGATSFQV |
| | | | DLD |
| 22 | Phosphoglycerate | Mycobacterium | MSLPVVLIADKLAPSTVAALGDQVEVRWVDGPDRDK |
| | dehydrogenase | tuberculosis | LLAAVPEADALLVRSATTVDAEVLAAAPKLKIVARAGV |
| | | | GLDNVDVDAATARGVLVVNAPTSNIHSAAEHALALLL |
| | | | AASRQIPAADASLREHTWKRSSFSGTEIFGKTVGVVGL |
| | | | GRIGQLVAQRIAAFGAYVVAYDPYVSPARAAQLGIELL |
| | | | SLDDLLARADFISVHLPKTPETAGLIDKEALAKTKPGVII |
| | | | VNAARGGLVDEAALADAITGGHVRAAGLDVFATEPC |
| | | | TDSPLFELAQVVVTPHLGASTAEAQDRAGTDVAESVR |
| | | | LALAGEFVPDAVNVGGGVVNEEVAPWLDLVRKLGVL |
| | | | AGVLSDELPVSLSVQVRGELAAEEVEVLRLSALRGLFSA |
| | | | VIEDAVTFVNAPALAAERGVTAEICKASESPNHRSVVD |
| | | | VRAVGADGSVVTVSGTLYGPQLSQKIVQINGRHFDLR |
| | | | AQGINLIIHYVDRPGALGKIGTLLGTAGVAIQAAQLSED |
| | | | AEGPGATILLRLDQDVPDDVRTAIAAAVDAYKLEVVDL |
| | | | S* |
| 23 | Glyceraldehyde- | Clostridium | MAKIAINGFGRIGRLALRRILEVPGLEVVAINDLTDAK |
| | 3-phosphate | acetobutylicum | MLAHLFKYDSSQGRFNGEIEVKEGAFVVNGKEVKVFA |
| | dehydrogenase | (strain ATCC 824/ | EADPEKLPWGDLGIDVVLECTGFFTKKEKAEAHVRAG |
| | | DSM 792/JCM | AKKVVISAPAGNDLKTIVFNVNNEDLDGTETVISGASC |
| | | 1419/LMG | TTNCLAPMAKVLNDKFGIEKGFMTTIHAFTNDQNTLD |
| | | 5710/VKM B- | GPHRKGDLRRARAAAVSIIPNSTGAAKAISQVIPDLAG |
| | | 1787) | KLDGNAQRVPVPTGSITELVSVLKKKVTVEEINAAMKE |
| | | | AADESFGYTEDPIVSADVVGINYGSLFDATLTKIVDVN |
| | | | GSQLVKTAAWYDNEMSYTSQLVRTLAYFAKIAK |
| 24 | Glyceraldehyde- | Bacillus subtilis | MKVKVAINGFGRIGRMVFRKAMLDDQIQVVAINASY |
| | 3-phosphate | (strain 168) | SAETLAHLIKYDTIHGRYDKEVVAGEDSLIVNGKKVLLL |
| | dehydrogenase | | NSRDPKQLPWREYDIDIVVEATGKFNAKDKAMGHIEA |
| | | | GAKKVILTAPGKNEDVTIVMGVNEDQFDAERHVIISN |
| | | | ASCTTNCLAPVVKVLDEEFGIESGLMTTVHAYTNDQK |
| | | | NIDNPHKDLRRARACGESIIPTTTGAAKALSLVLPHLKG |
| | | | KLHGLALRVPVPNVSLVDLVVDLKTDVTAEEVNEAFKR |
| | | | AAKTSMYGVLDYSDEPLVSTDYNTNPHSAVIDGLTTM |
| | | | VMEDRKVKVLAWYDNEWGYSCRVVDLIRHVAARMK |
| | | | HPSAV |
| 25 | Glyceraldehyde- | Corynebacterium | MVPLIGRLHRNNNVVVSVFGRLLVNVSDIDIIKSHRYA |
| | 3-phosphate | glutamicum | RHIISKELPLESSLDILRELVD |
| | dehydrogenase | (Brevibacterium | MNLGTASIDLGQLAYSFEESERTDLRAFLEDALAPVIG |
| | | saccharolyticum) | AETDINPTDIVLYGFGRIGRLL |
| | | | ARILVSREALYDGARLRAIVVRKNGEEDLVKRASLLRRD |
| | | | SVHGGFDGTITTDYDNNIIWA |
| | | | NGTPIKVIYSNDPATIDYTEYGINDAVVVDNTGRWRD |
| | | | REGLSQHLKSKGVAKVVLTAPGK |
| | | | GDLKNIVYGINHTDITADDQIVSAASCTTNAITPVLKVI |
| | | | NDRYGVEFGHVETVHSFTNDQ |
| | | | NLIDNFHKGSRRGRAAGLNMVLTETGAAKAVSKALPE |
| | | | LEGKLTGNAIRVPTPDVSMAVLN |
| | | | LTLKTEVDRDEVNEFLRRVSLHSDLRQQIDWIRSPEVV |
| | | | STDFVGTTHAGIVDGLATIATG |
| | | | RHLVLYVWYDNEFGYSNQVIRIVEEIAGVRPRVYPERK |
| | | | QPAVL |
| 26 | Glyceraldehyde- | Arabidopsis | MASVTFSVPKGFTEFSGLRSSSASLPFGKKLSSDEFVSIV |
| | 3-phosphate | thaliana | SFQTSAMGSSGGYRKGVTEAKLKVAINGFGRIGRNFL |
| | dehydrogenase | (Mouse-ear | RCWHGRKDSPLDIIAINDTGGVKQASHLLKYDSTLGIF |
| | | cress) | DADVKPSGETAISVDGKIIQVVSNRNPSLLPWKELGIDI |
| | | | VIEGTGVFVDREGAGKHIEAGAKKVIITAPGKGDIPTYV |
| | | | VGVNADAYSHDEPIISNASCTTNCLAPFVKVLDQKFGII |
| | | | KGTMTTTHSYTGDQRLLDASHRDLRRARAAALNIVPT |
| | | | STGAAKAVALVLPNLKGKLNGIALRVPTPNVSVVDLVV |
| | | | QVSKKTFAEEVNAAFRDSAEKELKGILDVCDEPLVSVD |
| | | | FRCSDFSTTIDSSLTMVMGDDMVKVIAWYDNEWGYS |
| | | | QRVVDLADIVANNWK |
| 27 | Glyceraldehyde- | Chlamydomonas | MAAMMQKSAFTGSAVSSKSGVRAKAARAVVDVRAE |
| | 3-phosphate | reinhardtii | KKIRVAINGFGRIGRNFLRCWHGRQNTLLDVVAINDS |
| | dehydrogenase | (Chlamydomonas | GGVKQASHLLKYDSTLGTFAADVKIVDDSHISVDGKQI |
| | | smithii) | KIVSSRDPLQLPWKEMNIDLVIEGTGVFIDKVGAGKHI |
| | | | QAGASKVLITAPAKDKDIPTFVVGVNEGDYKHEYPIISN |
| | | | ASCTTNCLAPFVKVLEQKFGIVKGTMTTTHSYTGDQRL |
| | | | LDASHRDLRRARAAALNIVPTTTGAAKAVSLVLPSLKG |
| | | | KLNGIALRVPTPTVSVVDLVVQVEKKTFAEEVNAAFRE |
| | | | AANGPMKGVLHVEDAPLVSIDFKCTDQSTSIDASLTM |
| | | | VMGDDMVKVVAWYDNEWGYSQRVVDLAEVTAKK |
| | | | WVA |
| 28 | Glyceraldehyde- | Paracoccus yeei | MTVKVAINGFGRIGRLALRSIVELRREDVEVVAINDLG |
| | 3-phosphate | | PVATLAHLLRYDSVHGPFRGSIDLGEDWIDVGSGKIRV |
| | dehydrogenase | | SSERDPVNLPHDELGVDVALECTGFFTSREKAESHIRA |
| | | | GAKRVLVSAPSDGADKTIVFKVNHDTLTADDVVVSNG |
| | | | SCTTNVLAPIAKVLNDLCGIERGYMTTVHAYTGDQPT |
| | | | QDTVHKDLYRARAAALSMIPTSTGAANAIGRVLPELK |
| | | | GKLHGSSIRVPTPNVSVIDLAVSTSREVSIDEVNDAIRQ |
| | | | TAAGAMSGVLAFTTEPLVSVDLNHRTESSIVALPQTDV |
| | | | VDGRMVRVLAWYDNEWSFATRMADTAVAMAKLI |
| 29 | Glyceraldehyde- | Celeribacter | MTIKLGINGFGRIGRCTLAHIAEAGLNEVEVVKINATG |
| | 3-phosphate | marinus | PIETNAHLLKYDSVHGRFGGPVAVNGNTMDLGRGPI |
| | dehydrogenase | | QVMSTYEPDELDWEGVDVVLECTGKFNDGHRADVH |
| | | | MRRGAKKVLISAPAKNVDRTVVFGVNHRDLRDDERM |
| | | | VSNGSCTTNCLAPLAKVLNDAVGIESGIMTTIHSYTGD |
| | | | QPTLDRRHPDLYRARAAALAMIPTSTGAAKALGEVLP |
| | | | ELSGKLDGTSMRVPTPNVSAVDLTFQAGKDVSVADIN |
| | | | EIVREAAMGHMGMVLGYDPEPKVSIDFNHTTESCIFA |
| | | | PEQTKVIGRTVRVLAWYDNEWGFSARMADVAAAM |
| | | | GRLSH |
| 30 | Glyceraldehyde- | Bradyrhizobiaceae | MAVRVAINGFGRIGRNVLRAIYESKRTDIEVVAINDLG |
| | 3-phosphate | bacterium | PVETNAHLLRFDSVHGRFPGEVTVEGDSIDIGNGKIKV |
| | dehydrogenase | SG-6C | TAVRDPATLPWKDLGIDIALECTGIFSAKAKASAHLTA |
| | | | GAKRVLVSAPSDGADATIVYGVNHDKLTKDHLVVSNG |
| | | | SCTTNCLAPIAKVLNDTVGIETGFMTTIHAYTGDQPTL |
| | | | DTMHSDLYRGRAAALSMIPTSTGAAKAIGLVLPELQG |
| | | | KLDGVAIRVPTPNVSVVDLKIVAKKTITKEEINDAMKR |
| | | | AASQELKGILGTTSHPNVSIDFNHDAHSSTFAFDQTKV |
| | | | QGGTLVRVLSWYDNEWGFSNRMADTAVAMSKLI |
| 31 | Glyceraldehyde- | Pseudomonas | MSGLLLIAGDCVRRHNVAPFFIPYMWKVLPVTQKPD |
| | 3-phosphate | furukawaii | QCLGEWIDREALAEAMIPMIGQLYRNNNVVTSIYGRG |
| | dehydrogenase | | LINRSVIAILKAHRFARHRLVDEAELSVHETFPILKAMSE |
| | | | LKLGAASVDLGKMVAKFKAEGNGRSVEQFTRDELAD |
| | | | VVGKQNGAAREGTDVVLYGFGRIGRLLARILIEKTGGG |
| | | | DGLRLRAIVVRKGAENDLVKRASLLRRDSVHGPFDGTI |
| | | | VIDEANNTLIANGNLIQVIYSNDPSSIDYTAYGIKNALLV |
| | | | DNTGKWRDAEGLSQHLKCPGIDRVVLTAPGKGALKNI |
| | | | VHGINHGDISADDKIISAASCTTNAIVPVLKAVNDQYGI |
| | | | VNGHVETVHSYTNDQNLIDNFHKGSRRGRSAPLNMV |
| | | | ITETGAATAAAKALPVLKGRLTGNAIRVPTPNVSMAIL |
| | | | NLNLEKATSREEINEYLRQMAMHSDLHKQIDYVSSQE |
| | | | VVSTDFVGSRHAGVVDAEATICNDNRVVLYVWYDNE |
| | | | FGYSCQVVRVMEDMAGVNPPAFPH |
| 32 | Glyceraldehyde- | Bigelowiella | MALVGAICFSLSGASNVAAPAMRTAPRVASMSRTMP |
| | 3-phosphate | natans | MSMAGQRVSVRPKMIQRREMKAEALSYTVDDIRGM |
| | dehydrogenase | (Pedinomonas | LGDDVANQILAKNTPTGPERNVVLYGFGRIGRLLARIIT |
| | | minutissima) | TIPTNLKLKGIVVRQPKKPDLEKRLELIKRDSVHGPFRG |
| | | (Chlorarachnion | TISMDEADNALIMNGQKVRLIYANQPEEVDYTKYGIN |
| | | sp. (strain | DAIVIDNTGVWRDDEGLAKHLSSKGAAQVILTAPGKG |
| | | CCMP621)) | VKNIVAGVNSDVIEGSDKILSAASCTTNAVVPALSVLH |
| | | | KKFGIVSGHIETVHSFTNDQNLIDNYHKADRRGRAGPL |
| | | | NMVISETGAGKAVAVALPELAGKLTASAIRVPTPDVSI |
| | | | AVMILDLEKSTTAEEINAAFKSEATGTLDDNLGYSDSKE |
| | | | AVSLDFIGSTHAGEVDALATKCTGNSCIVYVWYDNEN |
| | | | GYSNQVVRVVEQWAAKQEAAGLKFEAAEAMA |
| 33 | Glyceraldehyde- | Pseudomonas | MIFPDGVWLRSARQARIATQGTRLHNVAPFFSPYMW |
| | 3-phosphate | chlororaphis | KVPVTQKPDQCLGEWIDREALAEAMIPLIGQLYRNNN |
| | dehydrogenase | | VVSSIYGRSLINQSVIAILKAHRFARHRSSDDSELSVHET |
| | | | FPLLKAMSELKLGAASVDLGKLAFKFRNEGNGRTAEQ |
| | | | FVREELADVVGQQNASPRKGTDVVLYGFGRIGRLLARI |
| | | | LIEKTGGGDGLRLRAIVVRKGAENDLTKRASLLRRDSV |
| | | | HGSFNGTITIDEANNTITANGNLIQVIYAKNPTEVDYT |
| | | | QYGIKDALLVDNTGVWRDADGLGQHLACPGIDRVVL |
| | | | TAPGKGKLKNIVHGINHADITADDKIVSAASCTTNAIV |
| | | | PVLKAVNDKFGIVNGHVETVHSYTNDQNLIDNFHKG |
| | | | DRRGRSAALNMVITETGAATAAAKALPELAGKLTGNA |
| | | | IRVPTPNVSMAILNLNLEKAATREEMNEYLRYMALHS |
| | | | DLHKQIDFVNSQEVVSTDFVGSRHAGVVDAEATITQD |
| | | | NRVVLYVWYDNEFGYSCQVVRVMEDMAGVNPPAFPR |
| 34 | Glyceraldehyde- | Saccharolobus | MINVAVNGYGTIGKRVADAIIKQPDMKLVGVAKTSPN |
| | 3-phosphate | solfataricus | YEAFIAHRRGIRIYVPQQSIKKFEESGIPVAGTVEDLIKTS |
| | dehydrogenase | (strain ATCC | DIVVDTTPNGVGAQYKPIYLQLQRNAIFQGGEKAEVA |
| | | 35092/DSM | DISFSALCNYNEALGKKYIRVVSCNTTALLRTICTVNKVS |
| | | 1617/JCM | KVEKVRATIVRRAADQKEVKKGPINSLVPDPATVPSHH |
| | | 11322/P2) | AKDVNSVIRNLDIATMAVIAPTTLMHMHFINITLKDKV |
| | | (Sulfolobus | EKKDILSVLENTPRIVLISSKYDAEATAELVEVARDLKRD |
| | | solfataricus) | RNDIPEVMIFSDSIYVKDDEVMLMYAVHQESIVVPENI |
| | | | DAIRASMKLMSAEDSMRITNESLGILKGYLI |
| 35 | Glyceraldehyde- | Pseudomonas | ARKVIISAPASGADATIVYGVNHDTLRQSHQIISSASCT |
| | 3-phosphate | viridiflava | TNCLAPVAQVLHRELGIESGLMTTIHAYTNDQNLIDVY |
| | dehydrogenase | | HTDPYRARSATQSMIPSKTGAAEAVGLVLPELAGKLT |
| | | | GMAVRVPVINVSLVDLTVTLKRETTAEEVNALMKEAT |
| | | | QHSKVVG |
| 36 | Glyceraldehyde- | Kluyveromyces | MPDMTNESSSKPAQINIGINGFGRIGRLVLRAALTHPE |
| | 3-phosphate | lactis (Yeast) | VKVRLINNPSTTPEYAAYLFKYDSTHGKYRGEVEFDDE |
| | dehydrogenase | (Candida | RIIIQNDHVSAHIPLSHFREPERIPWASYNVDYVIDSTG |
| | | sphaerica) | VFKEVDTASRHKGVKKVIITAPSKTAPMYVYGVNHVK |
| | | | YNPLTDHVVSNASCTTNCLAPLVKALDDEFGIEEALMT |
| | | | TIHATTASQKTVDGTSSGGKDWRGGRSCQGNIIPSST |
| | | | GAAKAVGKILPELNGKITGMSIRVPTINISLVDLTFRTAK |
| | | | KTSYDDIMKALEQRSRSDMKGVLGVTKDAVVSSDFTS |
| | | | DSRSSIVDAKAGIELNDHFFKVLSWYDNEYGYSSRVVD |
| | | | LSIFMAQKDFEAGV |
| 37 | Glyceraldehyde- | Geobacillus | MALRVPTPNVSLVDLVVDVKRDVTVEEVNEALLRAAN |
| | 3-phosphate | stearothermo- | GPLKGILDFTMEPLVSIDFNTNPHEPAFGDH |
| | dehydrogenase | philus (Bacillus | |
| | | stearothermo- | |
| | | philus) | |
| 38 | NADP- | Streptococcus | LAISPFNYPVNLAGSKIAPALIAGNVVALKPPTQGSISG |
| | dependent non- | pyogenes | LLLAEAFAEAGIPAGVFNTITGRGSVIGDYIVEHEAVNFI |
| | phosphorylating | | NFTGSTPIGEGIGKLAGMRPIMLELGGKDSAIVLEDAD |
| | glyceraldehyde- | | LVLAAKNIVAGAFGYSGQRCTAVKRVLVMDKVADQL |
| | 3-phosphate | | AAEIKTLVEKLSVGMPEDDADITPLIDTSAADFVEGLIK |
| | dehydrogenase | | DATDKGATALTAFNREGNLISPVLFDHVTTDMRLAWE |
| | | | EPFGP |
| 39 | NADP- | Thermoproteus | MRAGLLEGVIKEKGGVPVYPSYLAGEWGGSGQEIEVK |
| | dependent non- | tenax (strain | SPIDLATIAKVISPSREEVERTLDVLFKRGRWSARDMP |
| | phosphorylating | ATCC 35583/ | GTERLAVLRKAADIIERNLDVFAEVLVMNAGKPKSAA |
| | glyceraldehyde- | DSM 2078/JCM | VGEVKAAVDRLRLAELDLKKIGGDYIPGDWTYDTLETE |
| | 3-phosphate | 92771 NBRC | GLVRREPLGVVAAITPFNYPLFDAVNKITYSFIYGNAVV |
| | dehydrogenase | 100435/Kra 1) | VKPSISDPLPAAMAVKALLDAGFPPDAIALLNLPGKEA |
| | | | EKIVADDRVAAVSFTGSTEVGERVVKVGGVKQYVMEL |
| | | | GGGDPAIVLEDADLDLAADKIARGIYSYAGQRCDAIKL |
| | | | VLAERPVYGKLVEEVAKRLSSLRVGDPRDPTVDVGPLI |
| | | | SPSAVDEMMAAIEDAVEKGGRVLAGGRRLGPTYVQP |
| | | | TLVEAPADRVKDMVLYKREVFAPVALAVEVKDLDQAI |
| | | | ELANGRPYGLDAAVFGRDVVKIRRAVRLLEVGAIYIND |
| | | | MPRHGIGYYPFGGRKKSGVFREGIGYAVEAVTAYKTIV |
| | | | FNYKGKGVWKYE |
| 40 | NADP- | Streptococcus | MTKQYKNYVNGEWKLSENEIKIYEPASGAELGSVPAM |
| | dependent non- | mutans | STEEVDYVYASAKKAQPAWRSLSYIERAAYLHKVADIL |
| | phosphorylating | serotype c | MRDKEKIGAVLSKEVAKGYKSAVSEVVRTAEIINYAAE |
| | glyceraldehyde- | (strain ATCC | EGLRMEGEVLEGGSFEAASKKKIAVVRREPVGLVLAIS |
| | 3-phosphate | 700610/ | PFNYPVNLAGSKIAPALIAGNVIAFKPPTQGSISGLLLAE |
| | dehydrogenase | UA159) | AFAEAGLPAGVFNTITGRGSEIGDYIVEHQAVNFINFT |
| | | | GSTGIGERIGKMAGMRPIMLELGGKDSAIVLEDADLEL |
| | | | TAKNIIAGAFGYSGQRCTAVKRVLVMESVADELVEKIR |
| | | | EKVLALTIGNPEDDADITPLIDTKSADYVEGLINDANDK |
| | | | GAAALTEIKREGNLICPILFDKVTTDMRLAWEEPFGPV |
| | | | LPIIRVTSVEEAIEISNKSEYGLQASIFTNDFPRAFGIAEQ |
| | | | LEVGTVHINNKTQRGTDNFPFLGAKKSGAGIQGVKYSI |
| | | | EAMTTVKSVVFDIK |
| 41 | NADP- | Alkalihalobacillus | MALQTEQFNANILRNGEWVESRTGERISISAPASGVA |
| | dependent non- | halodurans | LGSIPALSQEEVNDAIQGAKDAQKIWKIRPIHERVDLLY |
| | phosphorylating | (strain ATCC | AWADLLEERKEIIGELIMHEVAKPKKSAIGEVSRTADIIR |
| | glyceraldehyde- | BAA-125/DSM | HTADEALRLNGETLKGDQFKGGSSKKIALVEREPLGVV |
| | 3-phosphate | 18197/FERM | LAISPFNYPVNLAAAKIAPALVTGNTVVFKPATQGSLS |
| | dehydrogenase | 7344/JCM 9153/ | GIKMVEALADAGAPEGIIQVVTGRGSVIGDHLVEHPGI |
| | | C-125) (Bacillus | DMITFTGGTTTGERISEKAKMIPVVLELGGKDPAIVLD |
| | | halodurans) | DADLKLTASQIVSGAFSYSGQRCTAIKRVFVQDSVADQ |
| | | | LVANIKELVEQLTVGSPEDDADITPVIDEKSAAFIQGLID |
| | | | DALENGATLLSGNKRQGNLLSPTLLDDVTPAMRVAW |
| | | | EEPFGPVLPIIRVKDANEAISLSNQSDYGLQASIFTKDT |
| | | | DRAINIGKHLEVGTVHINAKTERGPDHFPFLGVKKSGL |
| | | | GVQGIKPSLLSMTRERVTVLNL |
| 42 | NADP- | Saccharolobus | MMSQEVIEVRSPSNLNVIGTVKRMHKDEVRGEIEEAY |
| | dependent non- | solfataricus | KGFEIISKMPLYKRTAILRKVSEILEREQERLAKLLAMEA |
| | phosphorylating | (strain ATCC | GKPIRDSRVEVLRASRLFRHAAEEVGMVLEGKNYRVD |
| | glyceraldehyde- | 35092/DSM | AYEYPPGNENRIVVSTREPIGVVTAILPFNFPINSFAHK |
| | 3-phosphate | 1617/JCM | VAPALAVGNSVVVKPSINTPLAAIEMKKILVEAGLPDS |
| | dehydrogenase | 11322/P2) | AVRVVTGYSSEIGDEIITHPLVGLITLTGSTQTGLKIASK |
| | | (Sulfolobus | AVSLGKRIIMELGGSDPIIILEDANIERASSIAVRARFEYA |
| | | solfataricus) | GQNCNAGKRIIVREEIYDKFVKAFNDKARALKVGDPLD |
| | | | ETTDVGPVINKESVENLNSVLADAKAKGGKVEILNKGP |
| | | | ESGSFFPLTMVTNPSLDMLVLKSEVFGPIVPIVSVKSDE |
| | | | EAIRIANSTEYGLQSAIFTNDVNRALKLSRELKFGAVIIN |
| | | | DSTRLRWDSLAFGGFKKSSIGREGVRETMLEMTENKLI |
| | | | AITLL |
| 43 | NADP- | Clostridium | LAISPFNYPVNLSGSKVAPALIAGNSVVLKPSTTGAISAL |
| | dependent non- | acetobutylicum | HLAEIFNAAGLPAGVLNTVTGKGSEIGDYLITHEEVNFI |
| | phosphorylating | | NFTGSSAVGKHISKIAGMIPMVLELGGKDAAIVLEDAN |
| | glyceraldehyde- | | LETTAKSIVSGAYGYSGQRCTAVKRVLVMDKVADELV |
| | 3-phosphate | | ELVTKKVKELKVGNPFDDVTITPLIDNKAADYVQTLIDD |
| | dehydrogenase | | AIEKGATLIVGNKRKENLMYPTLFDNVTADMRIAWEE |
| | | | PFGP |
| 44 | NADP- | Bacillus cereus | LAIAPFNYPVNLFAAKLAPALIMGNAVIFKPATQGAIS |
| | dependent non- | | GIKMVEALHKAGLPKGLVNVATGRGSVIGDYLVEHEG |
| | phosphorylating | | INMVSFTGGTNTGKHLAKKASMIPLVLELGGKDPGIV |
| | glyceraldehyde- | | REDADLQDAANHIVSGAFSYSGQRCTAIKRVLVHENV |
| | 3-phosphate | | ADELVGLLKEQVAKLSVGSPEQDSTIVPLIDDKSADFV |
| | dehydrogenase | | QGLVDDAVEKGATIVIGNKRERNLIYPTLIDHVTTDMR |
| | | | LAWEEPFAP |
| 45 | NADP- | Saccharolobus | MEKTSVLIKSKELMEIYELKDGVPYFKTYLSGQWISGDE |
| | dependent non- | solfataricus | WQDVISPIDLNIIGKIPKLNWNQIDDTLEHIYRKGRWSI |
| | phosphorylating | (strain ATCC | RDTPGEKRLDIYKKMASLLDKFKEDFVNVLMINNGKT |
| | glyceraldehyde- | 35092/DSM | KSAAEGEVKAAIERLLRADLDVKETRGDYVPGDWSSE |
| | 3-phosphate | 1617/JCM | TLETEAVVRKEPVGVVLSIVPFNYPLFDTVNKIVYTTVIG |
| | dehydrogenase | 11322/P2) | NAIIIKPPSSTPLPILMLAKVMELASFPKDSFAIITIPGRD |
| | | (Sulfolobus | MNKVVGDKRIQAISLTGSTETGEEVVRNAGIKQFIMEL |
| | | solfataricus) | GGGDPAIVLSDADLAWAAQRIAAGIISYTGQRCDSVK |
| | | | LVLVEEEVYDTLKDLLIKELTKSVKVGDPRDPLTTVGPVI |
| | | | DVKTVDEWEKAIKDAVEKGGKILFGGKRLGPTYIEPVLI |
| | | | EAPKETLKDMYFYNKEVFASAALLIKVKNIDEALEISNS |
| | | | RKYGLDAAIFGKDINKIRKLQRFLEVGAIYINDYPRHGI |
| | | | GYFPFGGRKDSGIGREGIGYTIQYVTAYKSIVYNYKGKG |
| | | | IWEYL |
| 46 | NADP- | Thermococcus | MDKLVHNKIFDGIYRPGEDGIPEFKAYVAGQWVFGES |
| | dependent non- | sibiricus (strain | FSEVKSPIDGRIIARVSRLSRDQVEEALARVYEKGREEIR |
| | phosphorylating | DSM 12597/ | NYPGEKRVQSFLKAAGLMREAFEDFVNVLILDAGKPK |
| | glyceraldehyde- | MM 739) | SNAVGETKATIERLEKTTFESRRMLGDYVPGDWSEETL |
| | 3-phosphate | | ESEGIVKREPYGLILAISPFNYPLFISAAKVVPALLSGNTV |
| | dehydrogenase | | LLKPASADPLAAIMFVRVLELAGFPKESFALLTIPGSFM |
| | | | DDVVKDRRIRAITFTGSTEVGEHIIRTGGIKAYHLELGG |
| | | | KDPAIVLDDANLELASEKIVKGMVSFSGQRCDAIRLILA |
| | | | QEKIYEDLKARILEKLKGMEPRNPLEDENAVMGPLIDE |
| | | | SSAEYIEEVYKDAVEKGARPLTEFRRKGNYVWPILLEVS |
| | | | KEKIKDLRAFNEDVFGSMALLIKVKDEDEAIEIAKSSRF |
| | | | GLDASVFSECERRARKVARKLEVGSVFINEYPRHGIGYY |
| | | | PFGGMKDSGVGREGIGYSVEQFTTTKTIVHNFKGYGV |
| | | | WEYL |
| 47 | Glucose-6- | Escherichia coli | MAVTQTAQACDLVIFGAKGDLARRKLLPSLYQLEKAG |
| | phosphate 1- | (strain K12) | QLNPDTRIIGVGRADWDKAAYTK |
| | dehydrogenase | | VVREALETFMKETIDEGLWDTLSARLDFCNLDVNDTA |
| | | | AFSRLGAMLDQKNRITINYFAMP |
| | | | PSTFGAICKGLGEAKLNAKPARVVMEKPLGTSLATSQE |
| | | | INDQVGEYFEECQVYRIDHYLG |
| | | | KETVLNLLALRFANSLFVNNWDNRTIDHVEITVAEEVG |
| | | | IEGRWGYFDKAGQMRDMIQNHL |
| | | | LQILCMIAMSPPSDLSADSIRDEKVKVLKSLRRIDRSNV |
| | | | REKTVRGQYTAGFAQGKKVPG |
| | | | YLEEEGANKSSNTETFVAIRVDIDNWRWAGVPFYLRT |
| | | | GKRLPTKCSEVVVYFKTPELNLF |
| | | | KESWQDLPQNKLTIRLQPDEGVDIQVLNKVPGLDHKH |
| | | | NLQITKLDLSYSETFNQTHLADA |
| | | | YERLLLETMRGIQALFVRRDEVEEAWKWVDSITEAWA |
| | | | MDNDAPKPYQAGTWGPVASVAMI |
| | | | TRDGRSWNEFE |
| 48 | 6- | Escherichia coli | MSKQQIGVVGMAVMGRNLALNIESRGYTVSIFNRSR |
| | phosphogluconate | (strain K12) | EKTEEVIAENPGKKLVPYYTVKEF |
| | dehydrogenase, | | VESLETPRRILLMVKAGAGTDAAIDSLKPYLDKGDIIID |
| | decarboxylating | | GGNTFFQDTIRRNRELSAEGF |
| | | | NFIGTGVSGGEEGALKGPSIMPGGQKEAYELVAPILTKI |
| | | | AAVAEDGEPCVTYIGADGAGH |
| | | | YVKMVHNGIEYGDMQLIAEAYSLLKGGLNLTNEELAQ |
| | | | TFTEWNNGELSSYLIDITKDIFT |
| | | | KKDEDGNYLVDVILDEAANKGTGKWTSQSALDLGEPL |
| | | | SLITESVFARYISSLKDQRVAAS |
| | | | KVLSGPQAQPAGDKAEFIEKVRRALYLGKIVSYAQGFS |
| | | | QLRAASEEYNWDLNYGEIAKIF |
| | | | RAGCIIRAQFLQKITDAYAENPQIANLLLAPYFKQIADD |
| | | | YQQALRDVVAYAVQNGIPVPT |
| | | | FSAAVAYYDSYRAAVLPANLIQAQRDYFGAHTYKRIDK |
| | | | EGVFHTEWLD |
| 49 | NADH oxidase | Lactobacillus | MKIISIKFVLGGNIMKVTVVGCTHAGTFAIKQILAEHPD |
| | | brevis ATCC | AEVTVYERNDVISFLSCGIALYLGGKVADPQGLFYSSPE |
| | | 14869 = DSM | ELQKLGANVQMNHNVLAIDPDQKTVTVEDLTSHAQT |
| | | 20054 | TESYDKLVMTSGSWPIVPKIPGIDSDRVKLCKNWAHA |
| | | | QALIEDAKEAKRITVIGAGYIGAELAEAYSTTGHDVTLI |
| | | | DAMDRVMPKYFDADFTDVIEQDYRDHGVQLALSETV |
| | | | ESFTDSATGLTIKTDKNSYETDLAILCIGFRPNTDLLKGK |
| | | | VDMAPNGAIITDDYMRSSNPDIFAAGDSAAVHYNPT |
| | | | HQNAYIPLATNAVRQGILVGKNLVKPTVKYMGTQSSS |
| | | | GLALYDRTIVSTGLTLAAAKQQGVNAEQVIVEDNYRPE |
| | | | FMPSTEPVLMSLVFDPDTHRILGGALMSKYDVSQSAN |
| | | | TLSVCIQNENTIDDLAMVDMLFQPNFDRPFNYLNILA |
| | | | QAAQAKVAQSVNA |
| 50 | NADH oxidase | Levilactobacillus | MKVTVVGCTHAGTFAIKQILAEHPDAEVTVYERNDVIS |
| | | brevis | FLSCGIALYLGGKVADPQGLFYSSPEELQKLGANVQM |
| | | (Lactobacillus | NHNVLAIDPDQKTVTVEDLTSHAQTTESYDKLVMTSG |
| | | brevis) | SWPIVPKIPGIDSDRVKLCKNWAHAQALIEDAKEAKRI |
| | | | TVIGAGYIGAELAEAYSTTGHDVTLIDAMDRVMPKYF |
| | | | DADFTDVIEQDYRDHGVQLALSETVESFTDSATGLTIK |
| | | | TDKNSYETDLAILCIGFRPNTDLLKGKVDMAPNGAIITD |
| | | | DYMRSSNPDIFAAGDSAAVHYNPTHQNAYIPLATNA |
| | | | VRQGILVGKNLVKPTVKYMGTQSSSGLALYDRTIVSTG |
| | | | LTLAAAKQQGVNAEQVIVEDNYRPEFMPSTEPVLMSL |
| | | | VFDPDTHRILGGALMSKYDVSQSANTLSVCIQNENTID |
| | | | DLAMVDMLFQPNFDRPFNYLNILAQAAQAKVAQSV |
| | | | NA |
| 51 | NADH oxidase | Secundilacto- | MKVAVIGCTHAGTFATKQILAENPDAEVTVYEKNDVIS |
| | | bacillus | FLSCGIALYLGGQVKDPQGLFYSNPDELSKLGATVLMR |
| | | silagincola | HNVTAIDNQAHTITVEDLATHEVTTSSYDKLVMTSGS |
| | | | WPIIPPIPGIDSDKVLLCKNWAHAQTLFQKAENAKRIA |
| | | | VIGAGYIGAELAEAYSLKGHDVTLIDAQDRVMPKYFDS |
| | | | AFTDKIEDEYRAHNVQLALSQKVQGFETTADGVTITTD |
| | | | KEAIDVDLAVLCIGFRPNTDLLAGKVDMAKNGAILTN |
| | | | DYMQTSDPDIFAAGDSAAVHYNPTGKNAYIPLATNA |
| | | | VRQGILVGKNIVKPTVKYMGTQSSSGLALYGTSLCSTG |
| | | | LTYEAAKAQGINAEQVIITDNYRPEFMPTTLPVTMSLV |
| | | | FNPDTREILGGALMSKYDISQSANLLSVCIQNHNTIDD |
| | | | LAMVDMLFQPNFDRPFNYLNILGQAAQEKLTTVKA |
| 52 | SidA/lucD/PvdA | Secundilacto- | MKVAVIGCTHAGTFATKQILAENPDAEVTVYERNDVI |
| | family | bacillus folii | SFLSCGIALYLGGEVKDPQGLFYSSPDELSGLGAKVLM |
| | monooxygenase | | RHDVKSIDNQAHTITVEDLDSHETTTTSYDKLVMTTGS |
| | | | WPVIPSIPGIKSDRVLLCKNWAHAQALFNRAKDSKRIA |
| | | | VIGAGYIGAELAEAYSLKNHDVTLIDAQDRVMPKYFD |
| | | | AAFTDKVEDEYTKHGVALALSQKVQSFETTDDAVTIHT |
| | | | DKESVTVDLAILCIGFKPNTILLEGKVDMAANGAILTND |
| | | | YMQSSDPDIFAAGDSAAVHYNPTGKNAYIPLATNAVR |
| | | | QGILVGKNLVKPTVKYMGTQSTSGLSLYGISLCSTGLTY |
| | | | EAAQEQGINAQQVIITDNYRPEFMPTTLPVTMSLVEN |
| | | | PETREILGGALMSKYDISQSANLLSVCIQNHNTIDDLA |
| | | | MVDMLFQPNFDRPFNYLNILGQAAQQKLAEAVKA |
| 53 | Nadh oxidase | Lactobacillus | MKVVIIGCTHAGTFATKQILTEHPDAEVTVYERNDVIS |
| | | kimchicus JCM | FLSCGIALYLGGQVKDPQGLFYSNPQELAELGATIKMR |
| | | 15530 | HDVKSVDNVAHTVVVEDLNTHETTTVLYDKLVMTTG |
| | | | SWPVIPPIPGIDSDRVKLCKNWVHAQTLFKEAPTAKRI |
| | | | AVIGAGYIGAELAEAYSLKGHEVTLIDGEDRVMPKYFD |
| | | | QTFTTKIEANYQNHGVRLALNQRVQSFETTTDAVTIHT |
| | | | DKDAITADLAILCIGFRPNTELVKGKVDMAQNGAILTN |
| | | | DYMQSSDPDIFAAGDAAAVHYNPTGKNAYIPLATNA |
| | | | VRQGILVGKNIAQPTVKYMGTQSSSGLALYGVSICSTG |
| | | | LTYEAAKAQGLAAEQVIVEDNYRPEFMPTTTPVLMSL |
| | | | VFDPNTRQILGGALMSDYDISQSANLLSVCIQNQNTID |
| | | | DLAMVDMLFQPNFDRPFNYLNILAQAAQEKVAKATA |
| | | | EA |
| 54 | NADH | Lentilactobacillus | MKIAVIGCTHAGTFAIKTVLAEHPDAEVTVYEKNDNIS |
| | peroxidase Npx | curieae | FLSCGIALYLGGEVKDPQGLFYSSPEELSKLGATVKMQ |
| | (EC 1.11.1.1) | | HSVESVDPDKKELTVKNLEDNTEFTDTYDKLVVTSGS |
| | | | WPVIPPIPGVDNKNVYLCKNYGHAKALFEKAKDAKRII |
| | | | VIGAGYIGAELAEAYSKSGHDVTLLDGADRVMPKYFD |
| | | | SEFTDVIEQDFRDHGVTLGLGEKVQEISGDDVATVKTD |
| | | | KNSYEADLVVLCVGFRPNNDLVKGKLDMTDNGALITN |
| | | | DYMQTSNPDIYAAGDSVAVHYNPTHGLAYIPLATNAV |
| | | | RQGILVGKNIETPTVKYIGTQSSSGLKLYDQIMVSTGLT |
| | | | LDSAKRAGINADRVVVKDNYRPEFMPTTAEVLMSLVY |
| | | | DKDSRKILGGSLMSTYDISQSANTLSVCIQDGDTIDELA |
| | | | MVDMLFQPQFDRPFNYLNILAQAAQAKADK |
| 55 | NADH oxidase | Lentilactobacillus | MKVAVIGCTHAGTFAIKTILAEHPDAEVTVYEKNDNIS |
| | | parakefiri | FLSCGIALYLGGHVKDPQGLFYSSPDELKKLGANVKM |
| | | | NHEVTNVDPASKTIEVSDLKNDQQFEDTYDKLVVTSG |
| | | | SWPVIPPIKGVDNKNVYLCKNWNHAKELFETAPEKKR |
| | | | ITIVGAGYIGAELAEAYNTKGHDVTLLDGADRIMSKYF |
| | | | DKEFTDVVEKDFTDHGVTLGLDQQVTEISGEDTVTIKT |
| | | | TKGSYETDLVVLCVGFRPNTGLFKGKLAMTDNGALIT |
| | | | NEYMQTSDPDIYGAGDSVAVHYNPTHGLAYIPLATNA |
| | | | VRQGILVGKNIEKPTVKYMGTQSSSGLKLYDKTIVSTGL |
| | | | TMAAAKAQNINASEVIITDNYRPEFMPTTEPVLMSLV |
| | | | YDPDSRRILGGSLLSTYDVSQSANTLSVCIQNENTIDDL |
| | | | AMVDMLFQPQFDRPFNYLNILGQAAQEQADKLTKAA |
| | | | SK |
| 56 | NADH oxidase | Lactobacillus | MKIAVIGCTHAGTFAVKTILSEHPDAQVTVYEKNDNIS |
| | (NADH | farraginis DSM | FLSCGIALYLGQQVKDPQGLFYSNPEELKTLGANVKM |
| | peroxidase Npx) | 18382 = JCM | NHEVTDVDPEAKTITVTNLTEGKQFTDTYDKLVATTGS |
| | | 14108 | WPIMPPIQGIDNKNVYLCKNWNHAQELFKTAPAKKRI |
| | | | TVVGAGYIGAELAEAYSTTGHDVTLIDGADRVMSKYF |
| | | | DRQFTDVVEQDYKDHGVTLALGQQVTGISGDDTVTIT |
| | | | TTKGSYETDLVIMCVGFKPNTSLFKGKLEMLKNGALIT |
| | | | NAYMQTSDPDIYAAGDSVAVHYNPTNSPAYIPLATNA |
| | | | VRQGILVGKNIEQPTVKYMGTQSSSGLKLYDRTIVSTG |
| | | | LTMAAANQQGINAAVVTITDNYRPEFMPTTEPVLMS |
| | | | LVYNPENRQILGGTLMSTYDVSQSANTLSVCIQNQNTI |
| | | | DDLAMVDMLFQPQFDRPFNYLNILGQAAQQQLAQQ |
| | | | ADSVKA |
| 57 | NAD-specific | Staphylococcus | MTENNNLVTSTQGIIKEALHKLGFDEGMYDLIKEPLR |
| | glutamate | aureus (strain | MLQVRIPVRMDDGTVKTFTGYRAQHNDAVGPTKGG |
| | dehydrogenase | Mu50/ATCC | VRFHPDVDEEEVKALSMWMTLKCGIVNLPYGGGKGG |
| | (NAD-GDH) (EC | 700699) | IVCDPRQMSIHEVERLSRGYVRAISQFVGPNKDIPAPD |
| | 1.4.1.2) | | VFTNSQIMAWMMDEYSALDKFNSPGFITGKPIVLGG |
| | | | SHGRDRSTALGVVIAIEQAAKRRNMQIEGAKVVIQGF |
| | | | GNAGSFLAKFLYDLGAKIVGISDAYGALHDPNGLDIDY |
| | | | LLDRRDSFGTVTNLFEETISNKELFELDCDILVPAAISNQ |
| | | | ITEDNAHDIKASIVVEAANGPTTPEATRILTERGILLVPD |
| | | | VLASAGGVTVSYFEWVQNNQGYYWSEEEVNEKLREK |
| | | | LEAAFDTIYELSQNRKIDMRLAAYIIGIKRTAEAARYRG |
| | | | WA |
| 58 | Catabolic NAD- | Bacillus subtilis | MSAKQVSKDEEKEALNLFLSTQTIIKEALRKLGYPGDM |
| | specific | (strain 168) | YELMKEPQRMLTVRIPVKMDNGSVKVFTGYRSQHND |
| | glutamate | | AVGPTKGGVRFHPEVNEEEVKALSIWMTLKCGIANLP |
| | dehydrogenase | | YGGGKGGIICDPRTMSFGELERLSRGYVRAISQIVGPT |
| | RocG (NAD- | | KDIPAPDVYTNSQIMAWMMDEYSRLREFDSPGFITG |
| | GDH) (EC | | KPLVLGGSQGRETATAQGVTICIEEAVKKKGIKLQNARI |
| | 1.4.1.2) | | IIQGFGNAGSFLAKFMHDAGAKVIGISDANGGLYNPD |
| | (Glutamate | | GLDIPYLLDKRDSFGMVTNLFTDVITNEELLEKDCDILV |
| | dehydrogenase) | | PAAISNQITAKNAHNIQASIVVEAANGPTTIDATKILNE |
| | (GlutDH) | | RGVLLVPDILASAGGVTVSYFEWVQNNQGYYWSEEE |
| | (Trigger enzyme | | VAEKLRSVMVSSFETIYQTAATHKVDMRLAAYMTGIR |
| | RocG) | | KSAEASRFRGWV |
| 59 | NAD-specific | Pseudomonas | MAFFTAASKADFQHQLQTALAQHLGDKALPQVTLFA |
| | glutamate | aeruginosa | EQFFSLISLDELTQRRLSDLVGCTLSAWRLLERFDRDQP |
| | dehydrogenase | (strain ATCC | EVRVYNPDYEKHGWQSTHTAVEVLHPDLPFLVDSVR |
| | (NAD-GDH) (EC | 15692/DSM | MELNRRGYSIHTLQTNVLSVRRSAKGELKEILPKGSQG |
| | 1.4.1.2) | 22644/CIP | KDVSQESLMYLEIDRCAHAGELRALEKAILEVLGEVRVT |
| | (NAD(+)- | 104116/JCM | VADFEPMKAKARELLTWLGKAKLKVPAEELKEVRSYLE |
| | dependent | 14847/LMG | WLLDNHFTFLGYEEFSVADEADGGRMVYDEKSFLGLT |
| | glutamate | 12228/1C/PRS | RLLRAGLSKDDLHIEDYAVAYLREPVLLSFAKAAHPSRV |
| | dehydrogenase) | 101/PAO1) | HRPAYPDYVSIRELDGKGRVIRECRFMGLFTSSVYNES |
| | | | VNDIPFIRGKVAEVMRRSGFDTKAHLGKELAQVLEVLP |
| | | | RDDLFQTPVDELFSTALAIVRIQERNKIRVFLRKDPYGR |
| | | | FCYCLAYVPRDVYSTETRLKIQQVLMERLQASDCEFWT |
| | | | FFSESVLARVQFILRVDPKSRIDIDPARLEEEVIQACRS |
| | | | WQDDYSSLVVENLGEAKGTNVLADFPKGFPAGYRER |
| | | | FAPHFAVVDLQHLLSLSEQRPLVMSFYQPLAQGEQQL |
| | | | HCKLYHADTPLALSDVLPILENLGLRVLGEFPYRLRHQN |
| | | | GREYWIHDFAFTYAEGLDVDIQQLNEILQDAFVHIVSG |
| | | | DAENDAFNRLVLTANLPWRDVALLRAYARYLKQIRLG |
| | | | FDLGYIASALNAHTDIARELVRLFKTRFYLARKLTAEDLE |
| | | | DKQQKLEQAILGALDEVQVLNEDRILRRYLDLIKATLRT |
| | | | NFYQPDGNGQNKSYFSFKFNPKAIPELPRPVPKYEIFV |
| | | | YSPRVEGVHLRGGKVARGGLRWSDREEDFRTEVLGLV |
| | | | KAQQVKNAVIVPVGAKGGFVPRRLPLGGSRDEIQAEA |
| | | | IACYRIFISGLLDITDNLKEGEVVPPANVVRHDEDDPYL |
| | | | VVAADKGTATFSDIANGIAAEYGFWLGDAFASGGSA |
| | | | GYDHKGMGITAKGAWVSVQRHFRERGIDVQKDNISV |
| | | | IGIGDMAGDVFGNGLLMSDKLQLVAAFNHMHIFIDP |
| | | | NPDAASSFVERQRLFNLPRSSWADYDAKLISAGGGIFL |
| | | | RSAKSIAITPEMKARFDIQADRLAPTELIHALLKAPVDLL |
| | | | WNGGIGTYVKSSKETHADVGDKANDGLRVDGRELRA |
| | | | KVVGEGGNLGMTQLARVEFGLHGGANNTDFIDNAG |
| | | | GVDCSDHEVNIKILLNEVVQAGDMTEKQRNALLVKM |
| | | | TDAVGALVLGNNYKQTQALSLAQRRARERIAEYKRLM |
| | | | GDLEARGKLDRALEFLPSDEELAERISAGQGLTRAELSV |
| | | | LISYSKIDLKESLLKSLVPDDDYLTRDMETAFPALLAEKF |
| | | | GDAMRRHRLKREIVSTQIANDLVNHMGITFVQRLKES |
| | | | TGMSAANVAGAYVIVRDVFHLPHWFRQIENLDYQVP |
| | | | ADIQLTLMDELMRLGRRATRWFLRSRRNELDAARDV |
| | | | AHFGPRIAALGLKLNELLEGPTRELWQARYQTYVDAG |
| | | | VPELLARMVAGTSHLYTLLPIIEASDVTGQDTAEVAKA |
| | | | YFAVGSALDLTWYLQQITNLPVENNWQALAREAFRD |
| | | | DLDWQQRAITVSVLQMQDGPKEVEARVGLWLEQHL |
| | | | PLVERWRAMLVELRAASGTDYAMYAVANRELMDLA |
| | | | QSSQHGVCIP |
| 60 | NAD-specific | Clostridium | MSKYVDRVIAEVEKKYADEPEFVQTVEEVLSSLGPVVD |
| | glutamate | symbiosum | AHPEYEEVALLERMVIPERVIEFRVPWEDDNGKVHVN |
| | dehydrogenase | (Bacteroides | TGYRVQFNGAIGPYKGGLRFAPSVNLSIMKFLGFEQAF |
| | (NAD-GDH) (EC | symbiosus) | KDSLTTLPMGGAKGGSDFDPNGKSDREVMRFCQAF |
| | 1.4.1.2) | | MTELYRHIGPDIDVPAGDLGVGAREIGYMYGQYRKIV |
| | | | GGFYNGVLTGKARSFGGSLVRPEATGYGSVYYVEAVM |
| | | | KHENDTLVGKTVALAGFGNVAWGAAKKLAELGAKAV |
| | | | TLSGPDGYIYDPEGITTEEKINYMLEMRASGRNKVQDY |
| | | | ADKFGVQFFPGEKPWGQKVDIIMPCATQNDVDLEQA |
| | | | KKIVANNVKYYIEVANMPTTNEALRFLMQQPNMVVA |
| | | | PSKAVNAGGVLVSGFEMSQNSERLSWTAEEVDSKLH |
| | | | QVMTDIHDGSAAAAERYGLGYNLVAGANIVGFQKIA |
| | | | DAMMAQGIAW |
| 61 | NAD-specific | Saccharomyces | MLFDNKNRGALNSLNTPDIASLSISSMSDYHVFDFPGK |
| | glutamate | cerevisiae | DLQREEVIDLLDQQGFIPDDLIEQEVDWFYNSLGIDDL |
| | dehydrogenase | (strain ATCC | FFSRESPQLISNIIHSLYASKLDFFAKSKFNGIQPRLFSIK |
| | (NAD-GDH) (EC | 204508/S288c) | NKIITNDNHAIFMESNTGVSISDSQQKNFKFASDAVG |
| | 1.4.1.2) | (Baker's yeast) | NDTLEHGKDTIKKNRIEMDDSCPPYELDSEIDDLFLDN |
| | | | KSQKNCRLVSFWAPESELKLTFVYESVYPNDDPAGVDI |
| | | | SSQDLLKGDIESISDKTMYKVSSNENKKLYGLLLKLVKE |
| | | | REGPVIKTTRSVENKDEIRLLVAYKRFTTKRYYSALNSLF |
| | | | HYYKLKPSKFYLESFNVKDDDIIIFSVYLNENQQLEDVLL |
| | | | HDVEAALKQVEREASLLYAIPNNSFHEVYQRRQFSPKE |
| | | | AIYAHIGAIFINHFVNRLGSDYQNLLSQITIKRNDTTLLEI |
| | | | VENLKRKLRNETLTQQTIINIMSKHYTIISKLYKNFAQIH |
| | | | YYHNSTKDMEKTLSFQRLEKVEPFKNDQEFEAYLNKFI |
| | | | PNDSPDLLILKTLNIFNKSILKTNFFITRKVAISFRLDPSLV |
| | | | MTKFEYPETPYGIFFVVGNTFKGFHIRFRDIARGGIRIV |
| | | | CSRNQDIYDLNSKNVIDENYQLASTQQRKNKDIPEGG |
| | | | SKGVILLNPGLVEHDQTFVAFSQYVDAMIDILINDPLKE |
| | | | NYVNLLPKEEILFFGPDEGTAGFVDWATNHARVRNCP |
| | | | WWKSFLTGKSPSLGGIPHDEYGMTSLGVRAYVNKIYE |
| | | | TLNLTNSTVYKFQTGGPDGDLGSNEILLSSPNECYLAIL |
| | | | DGSGVLCDPKGLDKDELCRLAHERKMISDFDTSKLSN |
| | | | NGFFVSVDAMDIMLPNGTIVANGTTFRNTFHTQIFKF |
| | | | VDHVDIFVPCGGRPNSITLNNLHYFVDEKTGKCKIPYIV |
| | | | EGANLFITQPAKNALEEHGCILFKDASANKGGVTSSSM |
| | | | EVLASLALNDNDFVHKFIGDVSGERSALYKSYVVEVQS |
| | | | RIQKNAELEFGQLWNLNQLNGTHISEISNQLSFTINKL |
| | | | NDDLVASQELWLNDLKLRNYLLLDKIIPKILIDVAGPQS |
| | | | VLENIPESYLKVLLSSYLSSTFVYQNGIDVNIGKFLEFIGG |
| | | | LKREAEASA |
| 62 | Glutamate | Pyrococcus | MVEQDPYEIVIKQLERAAQYMEISEEALEFLKRPQRIVE |
| | dehydrogenase | furiosus (strain | VTIPVEMDDGSVKVFTGFRVQHNWARGPTKGGIRW |
| | (GDH) (EC | ATCC 43587/ | HPEETLSTVKALAAWMTWKTAVMDLPYGGGKGGIIV |
| | 1.4.1.3) | DSM 3638/JCM | DPKKLSDREKERLARGYIRAIYDVISPYEDIPAPDVYTNP |
| | | 8422/Vc1) | QIMAWMMDEYETISRRKTPAFGIITGKPLSIGGSLGRI |
| | | | EATARGASYTIREAAKVLGWDTLKGKTIAIQGYGNAG |
| | | | YYLAKIMSEDFGMKVVAVSDSKGGIYNPDGLNADEVL |
| | | | KWKNEHGSVKDFPGATNITNEELLELEVDVLAPAAIEE |
| | | | VITKKNADNIKAKIVAEVANGPVTPEADEILFEKGILQIP |
| | | | DFLCNAGGVTVSYFEWVQNITGYYWTIEEVRERLDKK |
| | | | MTKAFYDVYNIAKEKNIHMRDAAYVVAVQRVYQAM |
| | | | LDRGWVKH |
| 63 | Glutamate | Thermococcus | MVEIDPFEMAVQQLERAAQFMDISEEALEWLKRPMR |
| | dehydrogenase | kodakarensis | IVEVSVPVEMDDGSVKVFTGFRVQHNWARGPTKGGI |
| | (GDH) (EC | (strain ATCC | RWHPAETLSTVKALATWMTWKVAVVDLPYGGGKGG |
| | 1.4.1.3) | BAA-918/JCM | IIVDPKKLSEREQERLARSYIRAVYDVIGPWTDIPAPDV |
| | | 12380/KOD1) | YTNPKIMAWMMDEYETIMRRKGPAFGVITGKPPGV |
| | | (Pyrococcus | GGIVARMDATARGAAFTIREAAKALGWDDLKGKTIAI |
| | | kodakaraensis | QGYGNAGYYLHKIMSEEFGMKVVAVSDSKGGIYNPD |
| | | (strain KOD1)) | GLPPADEVLKWKKEHGSVKDMPGTQNITNEELLELEV |
| | | | DILAPSAIEGVITKENADNVKAKIVAEVANGPVTPEAD |
| | | | EILHEKGILQIPDFLCNAGGVTVSYFEWVQNINGFYWT |
| | | | VEETRKRLDDKMTKAFWDVFNTHKEKNIHMRDAAYV |
| | | | VAVSRVYEAMKHRGWVKK |
| 64 | Glutamate | Lactobacillus sp. | MSNAKSYIESVKDTIQRRDPNQTEFQEAAFTVLDTLEP |
| | dehydrogenase | | VLDKHPEYIKANLLERLVEPERAIQFRVPWQSDDGQF |
| | | | HVNRGFRVQFNSAIGPYKGGLRLHPSVSLDVVKFLGFE |
| | | | QVLKNSLTGLPIGGAKGGSDFNPKGKSDSEVMRFCQS |
| | | | FMTELQRHIGPDLDVPAGDIGVGGREIGFLFGAYKRL |
| | | | NGYQNGVLTGKGLTFGGSLARPEATGFGLLYYTHAML |
| | | | KALGDTLTNKKIKVSGAGNVATYAIQKATEYGATVITC |
| | | | SDSDGYVYDPNGIDFKLVKQIKEVERGRIKEYAQRVET |
| | | | AEYHDGSVWDFDINYDIALPCATQNEISKDQAKLIAKD |
| | | | RVKYVCEGANMPSVPDAIATYRDNNIYYGPAKAANA |
| | | | GGVAVSALEMSQNSERLSWSFETVDNRLHDIMDNIF |
| | | | EISEAADKEYQLNQDYQAGANIAGFEKVAQTMMAQ |
| | | | GLV |
| 65 | Glutamate | Streptomyces | MQTKLDEAKAELLERAARVAENSPVGGHLPTGTADE |
| | dehydrogenase | sp. Act143 | GTPDRESVLAFLQRYYLHTAPEDLTDRDPVDVFGAALS |
| | (EC 1.4.1.2) | | HYRLAENRPQGTANVRVLTPTVEENGWACSHSIVEVV |
| | | | TDDMPFLVDSVTNELTRQGRGIHVVIHPQVVVRRDLT |
| | | | GQLIEVLPQRPSGDRSALPHDAHTESWIHVEIDRETDR |
| | | | ADLKQITADLLRVLSDVREAVEDWEKMREAALRIAED |
| | | | LPAEPTASDLPEQEVEEARELLRWLAADHFTFLGYREY |
| | | | QLREDDSLAAVPGTGLGILRSDPHHASEDSHPVSPSFE |
| | | | RLPADARAKAREHRLLVLTKANSRATVHRPSYLDYIGV |
| | | | KKFDADGNVVGERRFLGLFSSAAYTESVRRVPVIRRKV |
| | | | AEVLQRAGFSPNSHDGRDLLQILETYPRDELFQTPADE |
| | | | LQSIVTSVLYLQERRRLRLYLRQDEYGRYYSALVYLPRD |
| | | | RYTTGVRLRIIDILKEELGGISVDFTAWNTESILSRLHFV |
| | | | VRVPQGTELPELSDSDKERIEARLVEAARSWADGFAE |
| | | | ALTGELGEERAAELLRRYGNAFPEGYKADHTPRAAVA |
| | | | DLCHLEQLTEDSNFALSLYEPVGAAPEERRFKIYRKGEA |
| | | | VSLSAVLPVLSRLGVEVTDERPYELRCADRTASWIYDF |
| | | | GLRMPKAQNGAGDYLGDDGRERFQEAFAATWTGKA |
| | | | ENDGFNALVLSAGLTWRQAMVLRAYAKYLRQAGSTF |
| | | | SQDYMEDTLRNNVHTTRLLVSLFEARMSPDRQRAGH |
| | | | EIVDALLEEVDAALDQVASLDEDRILRSFLTVIKATLRTN |
| | | | FFQEALGGQPHDYVSMKFDPQAIPDLPAPRPAFEIWV |
| | | | YSPRVEGVHLRFGKVARGGLRWSDRREDFRTEILGLV |
| | | | KAQMVKNTVIVPVGAKGGFVAKQLPDPGVDREAWL |
| | | | AEGIASYKVFISALLDITDNMVAGEVVPPADVVRHDED |
| | | | DTYLVVAADKGTATFSDIANGVAESYNFWLGDAFASG |
| | | | GSAGYDHKGMGITARGAWESVKRHFRELDVDTQTE |
| | | | DFTVVGIGDMSGDVFGNGMLLSEHIRLVAAFDHRHIF |
| | | | IDPTPDAATSYAERRRVFELPRSSWADYNTELISAGGG |
| | | | VFPRSAKAIQLNAHIREALGIEGKVSKMTPADLMKAIL |
| | | | KAPVDLLWNGGIGTYVKSSAESHGDVGDKANDPIRV |
| | | | DGADLRVKVVGEGGNLGLTQLGRIEFALHGGKINTDA |
| | | | IDNSAGVDTSDHEVNIKILLNGLVTEGDMTVKQRNKLL |
| | | | AEMTDEVGHLVLRNNYAQNTAIANALAQSKDMLHA |
| | | | QQRFMRHLVREGHLDRALEFLPTDRQIRERLTNGQGL |
| | | | TGPETAVLLAYTKITVSDELLHTSLPDDPYLRTLLDAYFP |
| | | | SALRERFPEQMHGHPLAREIITTVLVNDTVNTGGTTYL |
| | | | HRLREETGASLEEIVRAQTAARAIFRSAAVWDGVESLD |
| | | | NRVEAAVQTRIRLHSRRLVERGTRWLLNNRPQPLQLA |
| | | | ETVEFFGERVEQVWSQLPKLLKGADLEWYQQIYDELS |
| | | | GAGVPDELATRVAGFSSAFPTLDIVSVADRNGKEPLDV |
| | | | AEVYYDLADRLHITQLMDRIIELPRADRWQSMARASI |
| | | | REDLYAAHAAVTADVLAVGNGTSTPEQRFKAWEEKN |
| | | | AAILGRARSTLEEIQGSETFDLANLSVAMRTMRTLLRT |
| | | | HS |
| 66 | Glutamate | Brevibacterium | MTQVSISDVAEEWRKLRGQGAPENFLDAYYPRFETG |
| | dehydrogenase | sp. S111 | ASDPEAYAAAAARHFAVGTEYDGQTPAIAIYNPAVDS |
| | (EC 1.4.1.2) | | PEFRDNHTVVALVLADMPHLVSSIVSDLAAAGRAIRR |
| | | | VHHPIITVSGSGSDLSIVSPAEAPSVTADTASLPQVSAA |
| | | | PDAQSSDDPQQQSWIRLEIDRIPEDDFAELEQRLREVL |
| | | | AYVAAAARDAQAMASKAKDIAAELSAHAPRPELASEA |
| | | | EAAAALLNWLDGHFTFLGYREYDYTHDAEHSSLEPIED |
| | | | TSMGISALRPLVKSPLSRAVAEKALEPHVLVLTKANSRS |
| | | | RVIRSSFMDYIGVKTFDSSGEIVGERRFVGVFKPEFYND |
| | | | SVLNIPVIDRKVSRILSASGFPAGSHSANELLGVLETYPR |
| | | | EDLLHEDTEEIFDVVMEIVDMQERRESRVFVRADPYQ |
| | | | RFVSVILYLPRDLYNTDARVRVQEVLREFYNADSVDFD |
| | | | VLLSESALARIHFVARMARDEQLPQISAEDVERRIVGA |
| | | | VRSWSEDVHAFLIPAEHGDSGTTNARADLWSKAFPPS |
| | | | YGEHHTPAEAVADVGRFEALDAGHGSAVRLYRPERSA |
| | | | DASVRLVLYRYERVGLSEVLPFLTAFGATVIDERPHELD |
| | | | LADGSHRYIYDFGLSFSEELGDDDCERISDAFMAGWE |
| | | | GKKEAGVFDRLIVTGLSWQHVTIIRALGKYLRQAGFTY |
| | | | SDAYVGEVYSDHPEISKLLVDYFAAKFDPAADDESREG |
| | | | AMAEIDDAIEAALSEVASLDADRVLRSSLELLRATLRTN |
| | | | YYIDESGELPTALVLKIRPTELSFVPKPKPALEMWVYSP |
| | | | QVEGAHLRFGAVARGGLRWSDRRDDFRTEVLGLVKA |
| | | | QMVKNALIVPTGAKGGFFPKQLPPMSDRDAWMAA |
| | | | GQAAYEVFIESMLEVSDDLTYGADDSSVVVHPDRVVR |
| | | | HDGDDYYLVVAADKGTARFSDVANAIAERRGFWLGD |
| | | | AFASGGSVGYDHKKMAITSRGAWKSVERHFRELGLN |
| | | | TAADDFTVVGIGDMSGDVFGNGMLRSEHIRLVAAFD |
| | | | HRDIFLDPNPDAARSFVERQRLFDLPRSSWQDYDRELI |
| | | | SAGGGVFSRSAKSIDLSAEAASVLGVDPGRRSPADLM |
| | | | SEILKAPVDLVYNGGIGTYIKASTESHAEVGDKSNDSIRI |
| | | | DGADVRARVIGEGGNLGVTQLGRVEAALNGVEINTD |
| | | | AVDNSAGVDSSDHEVNIKLLLRTLVHKGAFAAEEREQ |
| | | | VLHSFTDQVADRVLSNNYSQNVVLGEARAQTEAMSG |
| | | | TYGRLLNYLEKHADLDRAVEFLPNADELAKRRFGYYVS |
| | | | PELAVLLAYVKMHAADEILDSVVPDEMWMRRELVSY |
| | | | FPESLQEKYGELIPEHPLHREIATAKLVNRLVDRGGLTYI |
| | | | YRMLEETPASVPQIARVFVVVSEIFGLDDYFEAVCALD |
| | | | NEVPTEVQVKLQHDYVRLLDRSSRWLVQQAPDTLDV |
| | | | DSGIEMYGKVVEALRSRVPELVDGYDAESMQAKAQE |
| | | | YIDDGVPSDLAWRAAALLDEFVLLDITQLAARANESAE |
| | | | DVAEVYYAVNEHFAGSQVLTLIGGLDRSDRWSALARG |
| | | | SMRDDFYSAILSVAETVLAATDSPISGTPDERAKQRVA |
| | | | EWLERNETVVSRVIETTETILGLDSVTQAPLSVLLRSLRS |
| | | | MVRSSTWESEHNS |

### ITEMS OF THE DISCLOSURE

The present disclosure further provides the following embodiments and items:
Item 1. A genetically engineered bacterium which has been modified to have an increased expression of a polypeptide having D-3-phosphoglycerate dehydrogenase activity (serA) and a decreased production of hydroxyglutarate (HGA) compared to an otherwise identical bacterium that does not carry said modification.
Item 2. The bacterium according to item 1, wherein the hydroxyglutarate production is reduced by increasing cytosolic NADPH pool and/or reducing cytosolic NADH pool compared to an otherwise identical bacterium that does not carry said modification.
Item 3. The bacterium according to any of items 1-2, which expresses a polypeptide having D-3-phosphoglycerate dehydrogenase activity and which is selected from the group consisting of SEQ ID NOs: 1 to 22, and polypeptides comprising an amino acid sequence, which has at least about 70%, such as at least about 75%, at least about 80%, at least about 85%, at least about 90%, at least about 93%, at least 95%, at least 96%, at least 97%, at least 98%, at least 99%, or 100% sequence identity to any one of SEQ ID NOs: 1 to 22.
Item 4. The bacterium according to any one of items 1-3, wherein the decreased HGA production is achieved by expression of a polypeptide having D-3-phosphoglycerate dehydrogenase activity with reduced or no activity of HGA production.
Item 5. The bacterium according to any of items 1-4, which expresses a polypeptide having D-3-phosphoglycerate dehydrogenase activity and which is selected from the group consisting of SEQ ID NOs: 6 to 15, and polypeptides comprising an amino acid sequence, which has at least about 70%, such as at least about 75%, at least about 80%, at least about 85%, at least about 90%, at least about 93%, at least 95%, at least 96%, at least 97%, at least 98%, at least 99%, or 100% sequence identity to any one of SEQ ID NOs: 6 to 15.
Item 6. The bacterium according to any of items 1-5, which expresses a polypeptide having 3-phospho glycerate dehydrogenase activity and which polypeptide is derived from a polypeptide as defined in item 4 and rendered feed back insensitive by truncation of the C terminal domain (e.g. SEQ ID NO:16 and 18) or by site directed mutagenesis of key sites (e.g. SEQ ID NOs: 20-22).
Item 7. The bacterium according to item 1 to 6 wherein the bacterium has increased production of L-serine and or L-serine derived compounds.
Item 8. The bacterium according to any of items 4 and 7, wherein the resulting reduced growth and redox imbalance upon use of D-3-phosphoglycerate dehydrogenase activity with reduced or no activity of HGA production is solved by increasing cytosolic NADPH pool and/or reducing cytosolic NADH pool.
Item 9. The bacterium according to any one of items 1-8, in which the NADPH production is increased by heterologous expression of an NADPH dependent polypeptide having glyceraldehyde-3-phosphate dehydrogenase activity with or without additional ATP generation.
Item 10. The bacterium according to any of items 1-4, which expresses a NADPH dependent polypeptide having glyceraldehyde-3-phosphate dehydrogenase activity with or without additional ATP generation and which is selected from the group consisting of SEQ ID NOs: 23 to 46, and polypeptides comprising an amino acid sequence, which has at least about 70%, such as at least about Item 75%, at least about 80%, at least about 85%, at least about 90%, at least about 93%, at least 95%, at least 96%, at least 97%, at least 98%, at least 99%, or 100% sequence identity to any one of SEQ ID NOs: 23 to 46.
Item 11. The bacterium according to item 2, which has been modified to enhance the expression of the enzyme glucose-6-phosphate dehydrogenase (e.g. SEQ ID NO: 47) and/or 6-phosphogluconate dehydrogenase (e.g. SEQ ID NO: 48).
Item 12. The bacterium according to item 1-3, which expresses a heterologous polypeptide having NADH oxidase (Nox) activity to reduce the intracellular NADH pool.
Item 13. The bacterium according to item 12, wherein said heterologous polypeptide having NADH oxidase (Nox) activity is selected from the group consisting of SEQ ID NOs: 49 to 56, and polypeptides comprising an amino acid sequence, which has at least about 70%, such as at least about 75%, at least about 80%, at least about 85%, at least about 90%, at least about 93%, at least 95%, at least 96%, at least 97%, at least 98%, at least 99%, or 100% sequence identity to the amino acid sequence of any one of SEQ ID NOs: 49 to 56.
Item 14. The bacterium according to any one of items 1-2, which depletes NADH pool by expressing a heterologous NADH dependent polypeptide having glutamate dehydrogenase activity.
Item 15. The bacterium according to item 14, wherein the heterologous NADH dependent polypeptide having glutamate dehydrogenase activity is selected from the group consisting of SEQ ID NOs: 57 to 66, and polypeptides comprising an amino acid sequence, which has at least about 70%, such as at least about 75%, at least about 80%, at least about 85%, at least about 90%, at least about 93%, at least 95%, at least 96%, at least 97%, at least 98%, at least 99%, or 100% sequence identity to the amino acid sequence of any one of SEQ ID NOs: 57 to 66.
Item 16. The bacterium according to any one of items 1-15, which has been modified to have a decreased expression and/or activity of an endogenous NADH dependent polypeptide having Glyceraldehyde-3-phosphate dehydrogenase activity compared to an otherwise identical bacterium that does not carry said modification.
Item 17. The bacterium according to item 16, wherein the endogenous gene encoding said endogenous NADH dependent polypeptide having glyceraldehyde-3-phosphate dehydrogenase activity is inactivated.
Item 18. The bacterium according to any one of items 1-17, wherein said polypeptide having D-3-phosphoglycerate dehydrogenase activity is a polypeptide which has reduced activity towards alpha-ketoglutarate, where reduced activity is measured relative to activity of SEQ ID NO: 16.
Item 19. The bacterium according to item 18, wherein said polypeptide having D-3-phosphoglycerate dehydrogenase activity is selected from the group consisting of SEQ ID NOs: 16 to 22, and polypeptides comprising an amino acid sequence, which has at least about 70%, such as at least about 75%, at least about 80%, at least about 85%, at least about 90%, at least about 93%, at least 95%, at least 96%, at least 97%, at least 98%, at least 99%, or 100% sequence identity to the amino acid sequence of any one of SEQ ID NOs: 16 to 22.
Item 20. The bacterium according to any one of items 1-19, wherein said bacterium belongs to the Enterobacteriaceae family.
Item 21. The bacterium according to item 20, wherein said bacterium belongs to the genus Escherichia.
Item 22. The bacterium according to item 21, wherein said bacterium is Escherichia coli.
Item 23. The bacterium according to any one of items 1-19, wherein said bacterium belongs to the genus Corynebacterium.
Item 24. The bacterium according to item 23, wherein said bacterium is Corynebacterium glutamicum.
Item 25. A method for producing L-serine or a L-serine derivative, the method comprises cultivating a bacterium according to any one of items 1-24 in a culture medium.
Item 26. The method according to item 25, wherein the L-serine derivative is selected from the group consisting of L-cysteine, L-methionine, L-glycine, O-acetylserine, L-tryptophan, thiamine, ethanolamine and ethylene glycol.
Item 27. The method according to item 25 or 26, wherein the method further comprises isolating L-serine or the L-serine derivative from the culture medium.

1. A genetically engineered host cell producing a metabolite from 3-phosphoglycerate through a metabolic pathway, comprising one or more genetic modifications preventing or reducing or alleviating a negative impact on production of the metabolite from intracellular accumulation of NADH and/or hydroxy glutarate (HGA), produced by one or more pathway enzymes.

2. The host cell of claim 1 wherein the metabolite is L-serine or a derivative thereof.

3. The host cell of claim 1 or 2 comprising one or more, optionally two or more, optionally three or more, optionally four or more, optionally five or more, optionally seven genetic modifications selected from:
a) expression of a first heterologous NADPH generating enzyme converting glyceraldehyde 3-phosphate into 1,3-bisphosphoglycerate or a downstream precursor in the metabolite pathway;
b) expression of a heterologous enzyme converting NADH to NAD⁺;
c) expression of a heterologous enzyme in the metabolite pathway having a reduced or eliminated NADH consuming side activity compared to a corresponding pathway enzyme native to the host cell;
d) expression of a heterologous enzyme converting a side product of an enzyme in the metabolite pathway into a substrate of a metabolite pathway enzyme consuming NADH or NADPH;
e) overexpression of a native enzyme converting a side product of an enzyme in the metabolite pathway into a substrate of a metabolite pathway enzyme consuming NADH or NADPH;
f) expression of a second heterologous NADPH producing enzyme, which is not comprised in the metabolite pathway; and/or
g) overexpression of a native NADPH producing enzyme, which is not comprised in the metabolite pathway.

4. The host cell of claim 3 wherein the genetic modification comprises expression of the first heterologous NADPH generating enzyme producing a precursor for the metabolite pathway.

5. The host cell of claim 4 wherein the genetic modification comprises:
a) expression of the first heterologous NADPH generating enzyme converting glyceraldehyde 3-phosphate into 1,3-bisphosphoglycerate or a downstream precursor in the metabolite pathway; and
b) expression of the heterologous metabolite pathway enzyme having a reduced or eliminated NADH consuming side activity compared to a corresponding metabolite pathway enzyme native to the host cell.

6. The host cell of claim 5 wherein the genetic modification comprises:
a) expression of the first heterologous NADPH generating enzyme converting glyceraldehyde 3-phosphate into 1,3-bisphosphoglycerate or a downstream precursor in the metabolite pathway;
b) expression of the heterologous metabolite pathway enzyme having a reduced or eliminated NADH consuming side activity compared to a corresponding metabolite pathway enzyme native to the host cell; and
c) expression of the heterologous enzyme converting NADH to NAD⁺.

7. The host cell of claim 6 wherein the genetic modification comprises:
a) expression of the first heterologous NADPH generating enzyme converting glyceraldehyde 3-phosphate into 1,3-bisphosphoglycerate or a downstream precursor in the metabolite pathway;
b) expression of the heterologous metabolite pathway enzyme having a reduced or eliminated NADH consuming side activity compared to a corresponding metabolite pathway enzyme native to the host cell;
c) expression of the heterologous enzyme converting NADH to NAD⁺; and
d) expression of the heterologous enzyme and/or overexpression of the native enzyme converting the side product of the enzyme in the metabolite pathway into the substrate of the metabolite pathway enzyme consuming NADH or NADPH.

8. The host cell of claim 7 wherein the genetic modification comprises:
a) expression of the first heterologous NADPH generating enzyme converting glyceraldehyde 3-phosphate into 1,3-bisphosphoglycerate or a downstream precursor in the metabolite pathway;
b) expression of the heterologous metabolite pathway enzyme having a reduced or eliminated NADH consuming side activity compared to a corresponding metabolite pathway enzyme native to the host cell;
c) expression of the heterologous enzyme converting NADH to NAD⁺;
d) expression of the heterologous enzyme and/or overexpression of the native enzyme converting the side product of the enzyme in the metabolite pathway into a substrate of the metabolite pathway enzyme consuming NADH or NADPH; and
e) expression of the second heterologous NADPH producing enzyme and/or overexpression of the native NADPH producing enzyme, any of which are not comprised in the metabolite pathway.

9. The host cell of claims 3 to 8 wherein:
a) the first heterologous NADPH generating enzyme converting glyceraldehyde 3-phosphate into 1,3-bisphosphoglycerate or a downstream precursor in the metabolite pathway partially or completely replaces a NADH producing enzyme native to the host cell;
b) the heterologous metabolite pathway enzyme having a reduced or eliminated NADH consuming side activity, partially or completely replaces an enzyme native to the host cell;
c) the heterologous enzyme converting the side product of the enzyme in the metabolite pathway into the substrate of the metabolite pathway enzyme consuming NADH or NADPH partially or completely replaces a native enzyme converting the side product of the enzyme in the metabolite pathway into the substrate of the metabolite pathway enzyme consuming NADH or NADPH; and/or
d) the second heterologous enzyme producing NADPH partially or completely replaces a native NADPH producing enzyme, which is not comprised in the metabolite pathway.

10. The host cell of claim 9 wherein:
a) the first heterologous NADPH generating enzyme converting glyceraldehyde 3-phosphate into 1,3-bisphosphoglycerate or a downstream precursor in the metabolite pathway is bisphoshoglycerate synthase or a glyceraldehyde-3-phosphate dehydrogenase (GAPDH), both converting glyceraldehyde 3-phosphate into 1,3-bisphosphoglycerate; under the co-conversion of NADP⁺ into NADPH;
b) the heterologous enzyme converting NADH to NAD⁺ is a NADH oxidase (Nox);
c) the metabolite pathway enzyme having a reduced or eliminated NADH consuming side activity is a 3-phosphoglycerate dehydrogenase (PGDH) and the side activity is conversion of α-ketoglutarate (α-KGA) into α-hydroxyglutarate (α-HGA);
d) the enzyme converting the side product of the enzyme in the metabolite pathway into the substrate of a metabolite pathway enzyme is a glutamate dehydrogenase (GDH), the side product is α-ketoglutarate, and the substrate is glutamate; and/or
e) the second heterologous or the native NADPH producing enzyme, which are not comprised in the metabolite pathway is a Glucose-6-phosphate dehydrogenase and/or a 6-phosphogluconolactonase.

11. The host cell of claim 10 wherein heterologous enzymes are (i) enzymes from a species different from the host cell, (ii) a mutated enzymes from a species different from the host cell, and/or (iii) a mutated enzyme native to the host cell.

12. The host cell of claims 10 to 11 comprising a heterologous PGDH.

13. The host cell of claim 12 wherein the heterologous PGDH is overexpressed compared to a native PGDH having a side activity of converting α-ketoglutarate (α-KGA) into α-hydroxyglutarate.

14. The host cell of claim 13 wherein the heterologous PGDH is overexpressed compared to a native PGDH by 10 to 10.000%.

15. The host cell of claims 12 to 14 wherein the heterologous PGDH comprise a conserved region facing the active site which comprises the motif G/AXAGV, wherein the underlined X is a cationic residue positioned corresponding to position 129 of of the PGDH from Mycobacterium tuberculosis (SEQ ID NO: 10).

16. The host cell of claim 12 wherein the underlined X is selected from Arginine, Leucine or Histidine.

17. The host cell of claim 12 wherein the underlined X is Arginine.

18. The host cell of claims 12 to 15 wherein the heterologous PGDH comprise a conserved region facing the active site which does not comprises the motif GCFCI, wherein the underlined cysteine is positioned corresponding to position 129 of of the PGDH from Mycobacterium tuberculosis (SEQ ID NO: 10).

19. The host cell of claims 12 to 18 wherein the heterologous PGDH is mutant PGDH, optionally native to the host cell, modified to reduce or eliminate the NADH consuming side activity compared to the unmodified PGDH.

20. The host cell of claims 12 to 19 wherein the heterologous PGDH is a type I or type III PGDH, optionally a microbial type I or a type III PGDH.

21. The host cell of claims 12 to 20 wherein the heterologous PGDH enzymes is a SerA enzyme.

22. The host cell of claims 10 to 21 wherein:
a) the GAPDH enzyme comprises a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the GAP comprised in any one of SEQ ID NO: 23 to 46, optionally SEQ ID NO: 38 to 46;
b) the Nox comprises a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the Nox comprised in any one of SEQ ID NO: 49 to 56;
c) the PGDH comprises a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the PGDH comprised in any one of SEQ ID NO: 1 to 22;
d) the GDH comprises a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the GDH comprised in any one of SEQ ID NO: 57 to 66;
e) the Glucose-6-phosphate dehydrogenase comprises a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the Glucose-6-phosphate dehydrogenase comprised in SEQ ID NO: 47; and/or
f) the 6-phosphogluconolactonase comprises a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the 6-phosphogluconolactonase comprised in SEQ ID NO: 48.

23. The host cell of claim 22 comprising a PGDH comprising a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the PGDH comprised in any one of SEQ ID NO: 6 to 22.

24. The host cell of claim 23 wherein the PGDH comprises a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the PGDH comprised in any one of SEQ ID NO: 6 to 15.

25. The host cell of claim 23 wherein the PGDH comprises a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the PGDH comprised in SEQ ID NO: 14, or 19 to 21

26. The host cell of claims 22 to 25 wherein further comprising GAPDH comprising a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the GAPDH comprised in any one of SEQ ID NO: 38 to 46.

27. The host cell of claim 26 wherein the GAPDH comprises a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the GAPDH comprised in SEQ ID NO: 43.
The host cell of claims 22 to 27 comprising a PGDH comprises a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the PGDH comprised in anyone of SEQ ID NO: 14, or 19 to 21; ½ and a GAPDH comprising a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the GAPDH comprised in SEQ ID NO: 43

28. The host cell of claim any preceding claim further expressing one or more metabolite pathway enzymes selected from:
a) Phosphoserine aminotransferase (PSAT) converting 3-phosphohydroxypyruvate into phosphoserine; and
b) Phosphoserine phosphatase (PSPH) converting phosphoserine into L-serine.

29. The host cell of claim 28 wherein:
a) the PSAT is a serC and comprises a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the serC comprised in SEQ ID NO: 117; and
b) the PSPH is a serB and comprises a polypeptide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the serB comprised in SEQ ID NO: 118.

30. The host cell of any preceding claim, further comprising at least one transporter molecule facilitating transport of metabolite or any of its precursors.

31. The host cell of any preceding claim, wherein one or more native or endogenous genes of the cell is attenuated, disrupted and/or deleted.

32. The host cell of claim 31 wherein the native gene encodes a NADH dependent GAPDH or a @-HGA producing PHDH.

33. The host cell of any preceding claim, further comprising at least 2 copies of one or more polynucleotides encoding one or more metabolite pathway enzymes.

34. The host cell of any preceding claim further genetically modified to provide an increased amount of a substrate for one or more metabolite pathway enzymes.

35. The host cell of any preceding claim further genetically modified to exhibit increased tolerance towards one or more substrates, intermediates, or product molecules from the metabolite pathway.

36. The host cell of any preceding claim wherein the host cell is a prokaryotic cells, optionally a bacterium.

37. The host cell of claim 36 wherein the prokaryotic cell is a Pseudomonadota, optionally of the class gammaproteobacteria, optionally of the family Enterobacteriaceae, optionally of the genus Escherichia, optionally of the species Escherichia coli.

38. The host cell of claim 36 wherein the prokaryotic cell is an Actinomycetota, optionally of the class Actinobacteria, optionally of the family Corynebacteriaceae, optionally of the genus Corynebacterium, optionally of the species Corynebacterium glutamicum.

39. The host cell of claim 36 which has been further modified to have an increased expression of a polypeptide having D-3-phosphoglycerate dehydrogenase activity and a decreased production of α-hydroxyglutarate (α-HGA) compared to an otherwise identical bacterium that does not carry said modification.

40. The host cell of claim 39, wherein the α-hydroxyglutarate production is reduced by increasing cytosolic NADPH pool and/or reducing cytosolic NADH pool compared to an otherwise identical bacterium that does not carry said modification.

41. The host cell of any of claims 39 to 40, which expresses a polypeptide having D-3-phosphoglycerate dehydrogenase activity and which is selected from the group consisting of SEQ ID NOs: 1 to 22, and polypeptides comprising an amino acid sequence, which has at least about 70%, such as at least about 75%, at least about 80%, at least about 85%, at least about 90%, at least about 93%, at least 95%, at least 96%, at least 97%, at least 98%, at least 99%, or 100% sequence identity to any one of SEQ ID NOs: 1 to 22

42. The host cell of claim 41, which expresses a polypeptide having D-3-phosphoglycerate dehydrogenase activity and which is selected from the group consisting of SEQ ID NOs: 6 to 15, and polypeptides comprising an amino acid sequence, which has at least about 70%, such as at least about 75%, at least about 80%, at least about 85%, at least about 90%, at least about 93%, at least 95%, at least 96%, at least 97%, at least 98%, at least 99%, or 100% sequence identity to any one of SEQ ID NOs: 6 to 15.

43. The bacterium according to claims 39 to 42, wherein the resulting reduced growth and redox imbalance upon use of D-3-phosphoglycerate dehydrogenase activity with reduced or no activity of HGA production is solved by increasing cytosolic NADPH pool and/or reducing cytosolic NADH pool.

44. The host cell of any of claims 39 to 43, which expresses a NADPH dependent polypeptide having glyceraldehyde-3-phosphate dehydrogenase activity with or without additional ATP generation and which is selected from the group consisting of SEQ ID NOs: 38 to 46, and polypeptides comprising an amino acid sequence, which has at least about 70%, such as at least about 75%, at least about 80%, at least about 85%, at least about 90%, at least about 93%, at least 95%, at least 96%, at least 97%, at least 98%, or at least 99% sequence identity to any one of SEQ ID NOs: 38 to 46.

45. The host cell of claims 39 to 44, which expresses a heterologous polypeptide having NADH oxidase (Nox) activity to reduce the intracellular NADH pool.

46. The host cell of claim 45, wherein said heterologous polypeptide having NADH oxidase (Nox) activity is selected from the group consisting of SEQ ID NOs: 49 to 56, and polypeptides comprising an amino acid sequence, which has at least about 70%, such as at least about 75%, at least about 80%, at least about 85%, at least about 90%, at least about 93%, at least 95%, at least 96%, at least 97%, at least 98%, or at least 99% sequence identity to the amino acid sequence of any one of SEQ ID NOs: 49 to 56.

47. The host cell of any one of claims 39 to 46, which has been modified to have a decreased expression and/or activity of an endogenous NADH dependent polypeptide having Glyceraldehyde-3-phosphate dehydrogenase activity compared to an otherwise identical bacterium that does not carry said modification.

48. The bacterium of claim 47, wherein the endogenous gene encoding said endogenous NADH dependent polypeptide having glyceraldehyde-3-phosphate dehydrogenase activity is inactivated.

49. The bacterium of claim 48, wherein said polypeptide having D-3-phosphoglycerate dehydrogenase activity is selected from the group consisting of SEQ ID NOs: 16 to 22, and polypeptides comprising an amino acid sequence, which has at least about 70%, such as at least about 75%, at least about 80%, at least about 85%, at least about 90%, at least about 93%, at least 95%, at least 96%, at least 97%, at least 98%, or at least 99% sequence identity to the amino acid sequence of any one of SEQ ID NOs: 16 to 22.

50. The bacterium of any one of claims 39 to 49, wherein said bacterium belongs to the genus Escherichia or Corynebacterium.

51. A cell culture, comprising the host cell of any preceding claim and a growth medium.

52. A method for producing a metabolite from 3-phosphoglycerate comprising:
a) culturing the cell culture of claim 51 at conditions allowing the host cell to produce the metabolite; and
b) optionally recovering and/or isolating the metabolite.

53. The method of claim 52 wherein the metabolite is L-serine or a derivative thereof.

54. The method of claim 52, wherein the recovering and/or isolation step comprises separating a liquid phase of the cell or cell culture from a solid phase of the cell or cell culture to obtain a supernatant comprising the metabolite and subjecting the supernatant to one or more steps selected from:
a) disrupting the cells of the cell culture to release intracellular metabolites into the supernatant;
b) separating the supernatant from the solid phase of the cell culture, such as by filtration or gravity separation;
c) contacting the supernatant with one or more adsorbent resins in order to obtain at least a portion of the produced metabolite;
d) contacting the supernatant with one or more ion exchange or reversed-phase chromatography columns in order to obtain at least a portion of the metabolite; and
e) crystallizing or extracting the metabolite from the supernatant; and
f) evaporating the solvent of the supernatant to concentrate or precipitate the metabolite;
thereby recovering and/or isolating the metabolite.

55. The method of claims 52 to 54, further comprising one or more elements selected from:
a) culturing the cell culture in a nutrient medium;
b) culturing the cell culture under aerobic or anaerobic conditions
c) culturing the cell culture under agitation;
d) culturing the cell culture at a temperature of between 25 to 50 °C;
e) culturing the cell culture at a pH of between 3-9; and
f) culturing the cell culture for between 10 hours to 30 days.

56. The method of claims 52 to 55, wherein one or more steps of producing the metabolite is performed in vitro.

57. The method of claims 52 to 56, comprising feeding the cell culture with one or more metabolite precursors.

58. A fermentation composition comprising metabolites of the cell culture of claim 51 and the metabolite from 3-phosphoglycerate, wherein at least 20% by weight of the carbon is biobased.

59. The fermentation composition of claim 58, further comprising one or more compounds selected from trace metals, vitamins, salts, yeast nitrogen base, carbon source, YNB, and/or amino acids of the fermentation; wherein the concentration of the metabolite is at least 1 mg/kg composition.

60. The fermentation composition of claims 58 to 59, being substantially free of α-HGA.

61. The fermentation composition of claims 58 to 60 further comprising one or more carriers, agents, additives and/or excipients.


SEQUENCE LISTING (144 sequences)

SEQ ID NO: 1 - protein, 410 aa, Escherichia coli
MAKVSLEKDKIKFLLVEGVHQKALESLRAAGYTNIEFHKGALDDEQLKESIRDAHFIGLRSRTHLTEDVINAAEKLVAIGCFCIGTNQVDLDAAAKRGIPVFNAPFSNTRSVAELVIGELLLLLRGVPEANAKAHRGVWNKLAAGSFEARGKKLGIIGYGHIGTQLGILAESLGMYVYFYDIENKLPLGNATQVQHLSDLLNMSDVVSLHVPENPSTKNMMGAKEISLMKPGSLLINASRGTVVDIPALCDALASKHLAGAAIDVFPTEPATNSDPFTSPLCEFDNVLLTPHIGGSTQEAQENIGLEVAGKLIKYSDNGSTLSAVNFPEVSLPLHGGRRLMHIHENRPGVLTALNKIFAEQGVNIAAQYLQTSAQMGYVVIDIEADEDVAEKALQAMKAIPGTIRARLLY

SEQ ID NO: 2 - protein, 410 aa, Haemophilus influenzae
MTNKVSLDKSKIKFVLFEGVHQSALDTLHSAGYSNIDYYKKALDGDELKEAIKDAHFIGLRSRTHLTAEMIEAAPKLIAVGCFCIGTNQVDLNAAKARGIPVFNAPFSNTRSVAELVLGEILLLMRNVPQANAEVHRGVWNKSATGSYEVRGKKLGIIGYGHIGSQLSIIAESLGMDVYFYDIENKLPLGNAKQVRSLEELLSSCDVISLHVPELPSTKNLMNATRIAQLKQGAILINAARGTIVDIDALAQALKDGKIHGAAIDVFPVEPASINEEFVSPLREFDNVILTPHIGGSTAEAQENIGFEVAGKFVKYSDNGSTLSSVNFPEVSLPEHEGTKRLLHIHENRPGILNKLNQIFSEAHLNIAAQYLQTDPKIGYVVVDVETDDTSLLLTKLKEVDGTIRARVLY

SEQ ID NO: 3 - protein, 469 aa, Saccharomyces cerevisiae
MSYSAADNLQDSFQRAMNFSGSPGAVSTSPTQSFMNTLPRRVSITKQPKALKPFSTGDMNILLLENVNATAIKIFKDQGYQVEFHKSSLPEDELIEKIKDVHAIGIRSKTRLTEKILQHARNLVCIGCFCIGTNQVDLKYAASKGIAVFNSPFSNSRSVAELVIGEIISLARQLGDRSIELHTGTWNKVAARCWEVRGKTLGIIGYGHIGSQLSVLAEAMGLHVLYYDIVTIMALGTARQVSTLDELLNKSDFVTLHVPATPETEKMLSAPQFAAMKDGAYVINASRGTVVDIPSLIQAVKANKIAGAALDVYPHEPAKNGEGSFNDELNSWTSELVSLPNIILTPHIGGSTEEAQSSIGIEVATALSKYINEGNSVGSVNFPEVSLKSLDYDQENTVRVLYIHRNVPGVLKTVNDILSDHNIEKQFSDSHGEIAYLMADISSVNQSEIKDIYEKLNQTSAKVSIRLLY

SEQ ID NO: 4 - protein, 466 aa, Schizosaccharomyces pombe
MDIKGGRRGNVEDSLNKLSLSPPDNNSSFLSNHFQVRKSYSQAPARTLKPFASEDIKILLLENVNQSALSNLKDEGYQVEFLKTSMSEDDLVEKIKGVHAIGIRSKTRLTRRVLEAADSLIVIGCFCIGTNQVDLDFAAERGIAVFNSPYANSRSVAELVIGYIISLARQVGDRSLELHRGEWNKVSSGCWEIRGKTLGIIGYGHIGSQLSVLAEAMGLHVVYYDILPIMPLGSAKQLSSLPELLHRADFVSLHVPASPETKNMISSKEFAAMKEGSYLINASRGTVVDIPALVDASKSGKIAGAAIDVYPSEPAGNGKDKFVDSLNSWTSELTHCKNIILTPHIGGSTEEAQYNIGIEVSEALTRYINEGNSIGAVNFPEVSLRSLTEADRNAARVLFVHRNVPGVLRQVNELFIDHNIKSQFSDSRGDIAYLVADISDCTPGSLEALHQKLESLPCKINTRLLY

SEQ ID NO: 5 - protein, 466 aa, Neurospora crassa
MTVPREIAQPTGDKTAAQFSVSPSTFQSPISSSSFMASALAHRSAPKQLKPFNTEDVKILLLENVNQTGQDILRAQGYQVEFLKTSLPEDQLIEKIKDVHVIGIRSKTKLTANVLREAKNLLVIGCFCIGTNQVDLDYAAQHGIAVFNSPFANSRSVAELVIAEIITLARQLGDRSNEMHRGTWNKVSAKCWEIRGKTLGIVGYGHIGSQLSVLAEAMGMTVIYYDVVTLMAIGTARQVPTLEDLLAEADFVTLHVPETPETKNMISNDQFEKMKTGSYLINASRGTVVDIPALIKASRSGKIAGAALDVFPNEPAANGDYFTNDLNQWGEDLRGLNNIILTPHIGGSTEEAQRAIGVEVADALVRYINQGVTTGSVNVPEVTMRSLTLDEPNHARVIYIHRNVPGVLRKVNEILAEHNVDKQISDSKGDLAYLMADVSDVKQDDIKNIRDSLDSLSSRILTRILY

SEQ ID NO: 6 - protein, 299 aa, Entamoeba histolytica [sequence omitted]

SEQ ID NO: 7 - protein, 533 aa, Homo sapiens
MAFANLRKVLISDSLDPCCRKILQEGGLQVVEKQNLSKEELIAELQDCEGLIVRSATKVTADVINAAEKLQVVGRAGTGVDNVDLEAATRKGILVMNTPNGNSLSAAELTCGMIMCLARQIPQATASMKDGKWERKKFMGTELNGKTLGILGLGRIGREVATRMQSFGMKTIGYDPIISPEVSASFGVQQLPLEEIWPLCDFITVHTPLLPSTTGLLNDNTFAQCKKGVRVVNCARGGIVDEGALLRALQSGQCAGAALDVFTEEPPRDRALVDHENVISCPHLGASTKEAQSRCGEEIAVQFVDMVKGKSLTGVVNAQALTSAFSPHTKPWIGLAEALGTLMRAWAGSPKGTIQVITQGTSLKNAGNCLSPAVIVGLLKEASKQADVNLVNAKLLVKEAGLNVTTSHSPAAPGEQGFGECLLAVALAGAPYQAVGLVQGTTPVLQGLNGAVFRPEVPLRRDLPLLLFRTQTSDPAMLPTMIGLLAEAGVRLLSYQTSLVSDGETWHVMGISSLLPSLEAWKQHVTEAFQFHF

SEQ ID NO: 8 - protein, 533 aa, Rattus norvegicus
MAFANLRKILISDSLDPCCRKILQDGGLQVVEKQNLSKEELIAELQDCEGLIVRSATKVTADVINAAEKLQVVGRAGTGVDNVDLEAATRKGVLVMNTPNGNSLSAAELTCGMLMCLARQIPQATASMKDGKWDRKKFMGTELNGKTLGILGLGRIGREVAARMQAFGMKTVGYDPIISPEVAASFGVQQLPLEEIWPLCDFITVHTPLLPSTTGLLNDSTFAQCKKGVRVVNCARGGIVDEGALLRALQSGQCAGAALDVFTEEPPRDRALVDHENVISCPHLGASTKEAQSRCGEEIAVQFVDMVKGKSLTGVVNAQALTSAFSPHTKPWIGLAEALGTLMHAWAGSPKGTIQVVTQGTSLKNAGTCLSPAVIVGLLREASKQADVNLVNAKLLVKEAGLNVTTSHSPGVPGEQGIGECLLTVALAGAPYQAVGLVQGTTPMLQMLNGAVFRPEVPLRRGQPLLLFRAQPSDPVMLPTMIGLLAEAGVQLLSYQTSKVSDGDTWHVMGLSSLLPSLDAWKQHVSEAFQFCF

SEQ ID NO: 9 - protein, 533 aa, Mus musculus
MAFANLRKVLISDSLDPCCRKILQDGGLQVVEKQNLSKEELIAELQDCEGLIVRSATKVTADVINAAEKLQVVGRAGTGVDNVDLEAATRKGILVMNTPNGNSLSAAELTCGMIMCLARQIPQATASMKDGKWDRKKFMGTELNGKTLGILGLGRIGREVATRMQSFGMKTVGYDPIISPEVAASFGVQQLPLEEIWPLCDFITVHTPLLPSTTGLLNDSTFAQCKKGVRVVNCARGGIVDEGALLRALQSGQCAGAALDVFTEEPPRDRALVDHENVISCPHLGASTKEAQSRCGEEIAVQFVDMVKGKSLTGVVNAQALTSAFSPHTKPWIGLAEAMGTLMHAWAGSPKGTIQVVTQGTSLKNAGTCLSPAVIVGLLREASKQADVNLVNAKLLVKEAGLNVTTSHNPGVPGEQGSGECLLTVALAGAPYQAVGLVQGTTPMLQMLNGAVFRPEVPLRRGQPLLVFRAQPSDPGMLPTMIGLLAEAGVQLLSYQTSMVSDGEPWHVMGLSSLLPSLETWKQHVLEAFQFCF

SEQ ID NO: 10 - protein, 528 aa, Mycobacterium tuberculosis
MSLPVVLIADKLAPSTVAALGDQVEVRWVDGPDRDKLLAAVPEADALLVRSATTVDAEVLAAAPKLKIVARAGVGLDNVDVDAATARGVLVVNAPTSNIHSAAEHALALLLAASRQIPAADASLREHTWKRSSFSGTEIFGKTVGVVGLGRIGQLVAQRIAAFGAYVVAYDPYVSPARAAQLGIELLSLDDLLARADFISVHLPKTPETAGLIDKEALAKTKPGVIIVNAARGGLVDEAALADAITGGHVRAAGLDVFATEPCTDSPLFELAQVVVTPHLGASTAEAQDRAGTDVAESVRLALAGEFVPDAVNVGGGVVNEEVAPWLDLVRKLGVLAGVLSDELPVSLSVQVRGELAAEEVEVLRLSALRGLFSAVIEDAVTFVNAPALAAERGVTAEICKASESPNHRSVVDVRAVGADGSVVTVSGTLYGPQLSQKIVQINGRHFDLRAQGINLIIHYVDRPGALGKIGTLLGTAGVNIQAAQLSEDAEGPGATILLRLDQDVPDDVRTAIAAAVDAYKLEVVDLS

SEQ ID NO: 11 - protein, 525 aa, Bacillus subtilis
MFRVLVSDKMSNDGLQPLIESDFIEIVQKNVADAEDELHTFDALLVRSATKVTEDLFNKMTSLKIVGRAGVGVDNIDIDEATKHGVIVINAPNGNTISTAEHTFAMISSLMRHIPQANISVKSREWNRTAYVGSELYGKTLGIVGLGRIGSEIAQRARAFGMTVHVFDPFLTEERAKKIGVNSRTFEEVLESADIITVHTPLTKETKGLLNKETIAKTKKGVRLINCARGGIIDEAALLEALENGHVAGAALDVFEVEPPVDNKLVDHPLVIATPHLGASTKEAQLNVAAQVSEEVLQFAKGLPVMSAINLPAMTKDEFAKIKPYHQIAGKIGSLVSQCMKEPVQDVAIQYEGTIAKLETSFITKALLSGFLKPRVDSTVNEVNAGGVAKERGISFSEKISSSESGYDNCISVKVTGDRSTFTVTATYIPHFGERIVEINGFNIDFYPTGHLVYIQHQDTTGVIGRVGRILGDNDINIATMQVGRKEKGGEAIMMLSFDRHLEDKIVKELTNVPDIVSVKLIDLP

SEQ ID NO: 12 - protein, 523 aa, Methanobacterium sp.
MKVIIADSINEKGISDLKEVAEVTVATSITPEELVEVIKEYDAIVVRSRTKVTREVIEAGSKLKVIARAGVGVDNVDVQAATEKGIMVINAPESTSITVAEHSMGLMMSLARKISIADKSVKSGQWEKSKFMGIELNNKTLGIVGMGRIGSQVVIRAKAFNMEILVYDPYISKEAGAEMGVSVVDLETLLKESDVITIHVPLTPETRHLISTREMEIMKNTAFIINCARGGIVDEDALYYALKNDIIGGAALDVFESEPPQESPLLELDNLIATPHIGASTAEAQRDAALIVANEIITVFQGGSPRNVLNMPVMDSKNFKIMKPYLDLSEKMGSFMEQATSGNIKNMDVTYCGELSEMPQQDILTRTMLQGILNPILTEPVNLVNAPSIAQNRGIIVTEGKRCESEGYKCIIKLEIKTDKDEFSLDGTYVDEPKIIKINNYWVDVKPEGTMVIAKYQDLPGTIGAIGTKLGEHEINIATMQVGRKEVGGEAIMVLKVDQSVPQSVIDEVKKMDHVEDAVALEF

SEQ ID NO: 13 - protein, 624 aa, Arabidopsis thaliana
MAFSSSCSSVKAVNSRWTSPSPSPSSRFAVLPAFLHRRYATSVKLTAISAALKTVEQTTLTEDNRFSTVGSDSDEYNPTLPKPRILVTEKLGEAGVNLLREFGDVDCSYDLSPEDLKKKVAESDALIVRSGTKVTREVFEAAKGRLKVVGRAGVGIDNVDLQAATEHGCLVVNAPTANTVAAAEHGIALLASMARNVAQADASIKAGKWERSKYVGVSLVGKTLAVMGFGKVGTEVARRAKGLGMTVISHDPYAPADRARALGVDLVSFDQAISTADFVSLHMPLTPATKKVFNDETFSKMKKGVRLINVARGGVIDEDALVRALDAGIVAQAALDVFCEEPPSKDSRLIQHENVTVTPHLGASTKEAQEGVAIEIAEAVAGALKGELSATAVNAPMVAPEVLSELTPYIVLAEKLGRLAVQLASGGKGVQSIRVVYRSARDRDDLDTRLLRAMITKGIIEPISDSYVNLVNADFIAKQKGLRISEERMVVDSSPEYPVDSIQVQILNVESNFAGAVSDAGDISIEGKVKYGVPHLTCVGSFGVDVSLEGNLILCRQVDQPGMIGQVGNILGEQNVNVNFMSVGRTVLRKQAIMAIGVDEEPDNKTLERIGGVSAIEEFVFLKL

SEQ ID NO: 14 - protein, 530 aa, Corynebacterium glutamicum
MSQNGRPVVLIADKLAQSTVDALGDAVEVRWVDGPNRPELLDAVKEADALLVRSATTVDAEVIAAAPNLKIVGRAGVGLDNVDIPAATEAGVMVANAPTSNIHSACEHAISLLLSTARQIPAADATLREGEWKRSSFNGVEIFGKTVGIVGFGHIGQLFAQRLAAFETTIVAYDPYANPARAAQLNVELVELDELMSRSDFVTIHLPKTKETAGMFDAQLLAKSKKGQIIINAARGGLVDEQALADAIESGHIRGAGFDVYSTEPCTDSPLFKLPQVVVTPHLGASTEEAQDRAGTDVADSVLKALAGEFVADAVNVSGGRVGEEVAVWMDLARKLGLLAGKLVDAAPVSIEVEARGELSSEQVDALGLSAVRGLFSGIIEESVTFVNAPRIAEERGLDISVKTNSESVTHRSVLQVKVITGSGASATVVGALTGLERVEKITRINGRGLDLRAEGLNLFLQYTDAPGALGTVGTKLGAAGIAIEAAALTQAEKGDGAVLILRVESAVSEELEAEINAELGATSFQVDLD

SEQ ID NO: 15 - protein, 529 aa, Streptomyces sp.
MSSKPVVLIAEELSPATVDALGPDFEIRYCNGADRAELLPAIADVDAILIRSATKVDAEAIAAANRLKVVARAGVGLDNVDVPAATKAGVMVVNAPTSNIVTAAELACGLILSTARNIPQANAALKSGEWKRSKYTGVELAEKTLGVVGLGRIGALVAQRMSAFGMRIVAYDPYVQPARAAQMGVRMLSLDELLQVSDFITVHLPKTPETIGLIGEEALRKVKPSVRIINAARGGIVDEEALYSALKEGRVAGAGLDVYAKEPCTDSPLFEFDQVICTPHLGASTDEAQEKAGIAVAKSVRLALAGELVPDAVNVQGGVIAEDVKPGLPLAERLGRIFTALAGEVAVRLDVEVYGEITQHDVKVLELSALKGVFEDVVDETVSYVNAPLFAQERGVEVRLTTSSEATDHRNVVTVRGTLANGEEVSVSGTLSGPKHVQKIVAVGEYDVDLALADHMIVLRYEDRPGVVGTVGRILGEAGINIAGMQVARATAGGEALAVLTVDDTVSAAVLNELATEIGATSARSVNLV

SEQ ID NO: 16 - protein, 317 aa, Escherichia coli
MAKVSLEKDKIKFLLVEGVHQKALESLRAAGYTNIEFHKGALDDEQLKESIRDAHFIGLRSRTHLTEDVINAAEKLVAIGCFCIGTNQVDLDAAAKRGIPVFNAPFSNTRSVAELVIGELLLLLRGVPEANAKAHRGVWNKLAAGSFEARGKKLGIIGYGHIGTQLGILAESLGMYVYFYDIENKLPLGNATQVQHLSDLLNMSDVVSLHVPENPSTKNMMGAKEISLMKPGSLLINASRGTVVDIPALCDALASKHLAGAAIDVFPTEPATNSDPFTSPLCEFDNVLLTPHIGGSTQEAQENIGLEVAGKLIKYSD

SEQ ID NO: 17 - protein, 410 aa, Escherichia coli
MAKVSLEKDKIKFLLVEGVHQKALESLRAAGYTNIEFHKGALDDEQLKESIRDAHFIGLRSRTHLTEDVINAAEKLVAIGCFCIGTNQVDLDAAAKRGIPVFNAPFSNTRSVAELVIGELLLLLRGVPEANAKAHRGVWNKLAAGSFEARGKKLGIIGYGHIGTQLGILAESLGMYVYFYDIENKLPLGNATQVQHLSDLLNMSDVVSLHVPENPSTKNMMGAKEISLMKPGSLLINASRGTVVDIPALCDALASKHLAGAAIDVFPTEPATNSDPFTSPLCEFDNVLLTPHIGGSTQEAQENIGLEVAGKLIKYSDNGSTLSAVNFPEVSLPLHGGRRLMHIAEARPGVLTALNKIFAEQGVAIAAQYLQTSAQMGYVVIDIEADEDVAEKALQAMKAIPGTIRARLLY

SEQ ID NO: 18 - protein, 317 aa, Escherichia coli
MAKVSLEKDKIKFLLVEGVHQKALESLRAAGYTNIEFHKGALDDEQLKESIRDAHFIGLRSRTHLTEDVINAAEKLVAIGCFCIGTNQVDLDAAAKRGIPVFNAPFSNTRSVAELVIGELLLLLRGVPEANAKAHRGVWNKLAARSFEARGKKLGIIGYGHIGTQLGILAESLGMYVYFYDIENKLPLGNATQVQHLSDLLNMSDVVSLHVPENPSTKNMMGAKEISLMKPGSLLINASRGTVVDIPALCDALASKHLAGAAIDVFPTEPATNSDPFTSPLCEFDNVLLTPHIGGSTQEAQENIGLEVAGKLIKYSD

SEQ ID NO: 19 - protein, 337 aa, Corynebacterium glutamicum
MSQNGRPVVLIADKLAQSTVDALGDAVEVRWVDGPNRPELLDAVKEADALLVRSATTVDAEVIAAAPNLKIVGRAGVGLDNVDIPAATEAGVMVANAPTSNIHSACEHAISLLLSTARQIPAADATLREGEWKRSSFNGVEIFGKTVGIVGFGHIGQLFAQRLAAFETTIVAYDPYANPARAAQLNVELVELDELMSRSDFVTIHLPKTKETAGMFDAQLLAKSKKGQIIINAARGGLVDEQALADAIESGHIRGAGFDVYSTEPCTDSPLFKLPQVVVTPHLGASTEEAQDRAGTDVADSVLKALAGEFVADAVNVSGGRVGEEVAVWMDLARKLG

SEQ ID NO: 20 - protein, 530 aa, Corynebacterium glutamicum
MSQNGRPVVLIADKLAQSTVDALGDAVEVRWVDGPNRPELLDAVKEADALLVRSATTVDAEVIAAAPNLKIVGRAGVGLDNVDIPAATEAGVMVANAPTSNIHSACEHAISLLLSTARQIPAADATLREGEWKRSSFNGVEIFGKTVGIVGFGHIGQLFAQRLAAFETTIVAYDPYANPARAAQLNVELVELDELMSRSDFVTIHLPKTKETAGMFDAQLLAKSKKGQIIINAARGGLVDEQALADAIESGHIRGAGFDVYSTEPCTDSPLFKLPQVVVTPHLGGSTEEAQDRAGTDVADSVLKALAGEFVADAVNVSGGRVGEEVAVWMDLARKLGLLAGKLVDAAPVSIEVEARGELSSEQVDALGLSAVRGLFSGIIEESVTFVNAPRIAEERGLDISVKTNSESVTHRSVLQVKVITGSGASATVVGALTGLERVEKITRINGRGLDLRAEGLNLFLQATDAPGALGTVGTKLGAAGINIEAAALTQAEKGDGAVLILRVESAVSEELEAEINAELGATSFQVDLD

SEQ ID NO: 21 - protein, 530 aa, Corynebacterium glutamicum
MSQNGRPVVLIADKLAQSTVDALGDAVEVRWVDGPNRPELLDAVKEADALLVRSATTVDAEVIAAAPNLKIVGRAGVGLDNVDIPAATEAGVMVANAPTSNIHSACEHAISLLLSTARQIPAADATLREGEWKRSSFNGVEIFGKTVGIVGFGHIGQLFAQRLAAFETTIVAYDPYANPARAAQLNVELVELDELMSRSDFVTIHLPKTKETAGMFDAQLLAKSKKGQIIINAARGGLVDEQALADAIESGHIRGAGFDVYSTEPCTDSPLFKLPQVVVTPHLGASTEEAQDRAGTDVADSVLKALAGEFVADAVNVSGGRVGEEVAVWMDLARKLGLLAGKLVDAAPVSIEVEARGELSSEQVDALGLSAVRGLFSGIIEESVTFVNAPRIAEERGLDISVKTNSESVTHRSVLQVKVITGSGASATVVGALTGLERVEKITRINGRGLDLRAEGLNLFLQYTDAPGALGTVGTKLGAAGINIEAAALTQAEKGDGAVLILRVESAVSEELEAEINAELGATSFQVDLD

SEQ ID NO: 22 - protein, 528 aa, Mycobacterium tuberculosis
MSLPVVLIADKLAPSTVAALGDQVEVRWVDGPDRDKLLAAVPEADALLVRSATTVDAEVLAAAPKLKIVARAGVGLDNVDVDAATARGVLVVNAPTSNIHSAAEHALALLLAASRQIPAADASLREHTWKRSSFSGTEIFGKTVGVVGLGRIGQLVAQRIAAFGAYVVAYDPYVSPARAAQLGIELLSLDDLLARADFISVHLPKTPETAGLIDKEALAKTKPGVIIVNAARGGLVDEAALADAITGGHVRAAGLDVFATEPCTDSPLFELAQVVVTPHLGASTAEAQDRAGTDVAESVRLALAGEFVPDAVNVGGGVVNEEVAPWLDLVRKLGVLAGVLSDELPVSLSVQVRGELAAEEVEVLRLSALRGLFSAVIEDAVTFVNAPALAAERGVTAEICKASESPNHRSVVDVRAVGADGSVVTVSGTLYGPQLSQKIVQINGRHFDLRAQGINLIIHYVDRPGALGKIGTLLGTAGVAIQAAQLSEDAEGPGATILLRLDQDVPDDVRTAIAAAVDAYKLEVVDLS

SEQ ID NO: 23 - protein, 334 aa, Clostridium acetobutylicum
MAKIAINGFGRIGRLALRRILEVPGLEVVAINDLTDAKMLAHLFKYDSSQGRFNGEIEVKEGAFVVNGKEVKVFAEADPEKLPWGDLGIDVVLECTGFFTKKEKAEAHVRAGAKKVVISAPAGNDLKTIVFNVNNEDLDGTETVISGASCTTNCLAPMAKVLNDKFGIEKGFMTTIHAFTNDQNTLDGPHRKGDLRRARAAAVSIIPNSTGAAKAISQVIPDLAGKLDGNAQRVPVPTGSITELVSVLKKKVTVEEINAAMKEAADESFGYTEDPIVSADVVGINYGSLFDATLTKIVDVNGSQLVKTAAWYDNEMSYTSQLVRTLAYFAKIAK

SEQ ID NO: 24 - protein, 340 aa, Bacillus subtilis
MKVKVAINGFGRIGRMVFRKAMLDDQIQVVAINASYSAETLAHLIKYDTIHGRYDKEVVAGEDSLIVNGKKVLLLNSRDPKQLPWREYDIDIVVEATGKFNAKDKAMGHIEAGAKKVILTAPGKNEDVTIVMGVNEDQFDAERHVIISNASCTTNCLAPVVKVLDEEFGIESGLMTTVHAYTNDQKNIDNPHKDLRRARACGESIIPTTTGAAKALSLVLPHLKGKLHGLALRVPVPNVSLVDLVVDLKTDVTAEEVNEAFKRAAKTSMYGVLDYSDEPLVSTDYNTNPHSAVIDGLTTMVMEDRKVKVLAWYDNEWGYSCRVVDLIRHVAARMKHPSAV

SEQ ID NO: 25 - protein, 463 aa, Corynebacterium glutamicum
MVPLIGRLHRNNNVVVSVFGRLLVNVSDIDIIKSHRYARHIISKELPLESSLDILRELVDMNLGTASIDLGQLAYSFEESERTDLRAFLEDALAPVIGAETDINPTDIVLYGFGRIGRLLARILVSREALYDGARLRAIVVRKNGEEDLVKRASLLRRDSVHGGFDGTITTDYDNNIIWANGTPIKVIYSNDPATIDYTEYGINDAVVVDNTGRWRDREGLSQHLKSKGVAKVVLTAPGKGDLKNIVYGINHTDITADDQIVSAASCTTNAITPVLKVINDRYGVEFGHVETVHSFTNDQNLIDNFHKGSRRGRAAGLNMVLTETGAAKAVSKALPELEGKLTGNAIRVPTPDVSMAVLNLTLKTEVDRDEVNEFLRRVSLHSDLRQQIDWIRSPEVVSTDFVGTTHAGIVDGLATIATGRHLVLYVWYDNEFGYSNQVIRIVEEIAGVRPRVYPERKQPAVL

SEQ ID NO: 26 - protein, 396 aa, Arabidopsis thaliana
MASVTFSVPKGFTEFSGLRSSSASLPFGKKLSSDEFVSIVSFQTSAMGSSGGYRKGVTEAKLKVAINGFGRIGRNFLRCWHGRKDSPLDIIAINDTGGVKQASHLLKYDSTLGIFDADVKPSGETAISVDGKIIQVVSNRNPSLLPWKELGIDIVIEGTGVFVDREGAGKHIEAGAKKVIITAPGKGDIPTYVVGVNADAYSHDEPIISNASCTTNCLAPFVKVLDQKFGIIKGTMTTTHSYTGDQRLLDASHRDLRRARAAALNIVPTSTGAAKAVALVLPNLKGKLNGIALRVPTPNVSVVDLVVQVSKKTFAEEVNAAFRDSAEKELKGILDVCDEPLVSVDFRCSDFSTTIDSSLTMVMGDDMVKVIAWYDNEWGYSQRVVDLADIVANNWK

SEQ ID NO: 27 - protein, 374 aa, Chlamydomonas reinhardtii
MAAMMQKSAFTGSAVSSKSGVRAKAARAVVDVRAEKKIRVAINGFGRIGRNFLRCWHGRQNTLLDVVAINDSGGVKQASHLLKYDSTLGTFAADVKIVDDSHISVDGKQIKIVSSRDPLQLPWKEMNIDLVIEGTGVFIDKVGAGKHIQAGASKVLITAPAKDKDIPTFVVGVNEGDYKHEYPIISNASCTTNCLAPFVKVLEQKFGIVKGTMTTTHSYTGDQRLLDASHRDLRRARAAALNIVPTTTGAAKAVSLVLPSLKGKLNGIALRVPTPTVSVVDLVVQVEKKTFAEEVNAAFREAANGPMKGVLHVEDAPLVSIDFKCTDQSTSIDASLTMVMGDDMVKVVAWYDNEWGYSQRVVDLAEVTAKKWVA

SEQ ID NO: 28 - protein, 335 aa, Paracoccus yeei
MTVKVAINGFGRIGRLALRSIVELRREDVEVVAINDLGPVATLAHLLRYDSVHGPFRGSIDLGEDWIDVGSGKIRVSSERDPVNLPHDELGVDVALECTGFFTSREKAESHIRAGAKRVLVSAPSDGADKTIVFKVNHDTLTADDVVVSNGSCTTNVLAPIAKVLNDLCGIERGYMTTVHAYTGDQPTQDTVHKDLYRARAAALSMIPTSTGAANAIGRVLPELKGKLHGSSIRVPTPNVSVIDLAVSTSREVSIDEVNDAIRQTAAGAMSGVLAFTTEPLVSVDLNHRTESSIVALPQTDVVDGRMVRVLAWYDNEWSFATRMADTAVAMAKLI

SEQ ID NO: 29 - protein, 333 aa, Celeribacter marinus
MTIKLGINGFGRIGRCTLAHIAEAGLNEVEVVKINATGPIETNAHLLKYDSVHGRFGGPVAVNGNTMDLGRGPIQVMSTYEPDELDWEGVDVVLECTGKFNDGHRADVHMRRGAKKVLISAPAKNVDRTVVFGVNHRDLRDDERMVSNGSCTTNCLAPLAKVLNDAVGIESGIMTTIHSYTGDQPTLDRRHPDLYRARAAALAMIPTSTGAAKALGEVLPELSGKLDGTSMRVPTPNVSAVDLTFQAGKDVSVADINEIVREAAMGHMGMVLGYDPEPKVSIDFNHTTESCIFAPEQTKVIGRTVRVLAWYDNEWGFSARMADVAAAMGRLSH

SEQ ID NO: 30 - protein, 335 aa, Bradyrhizobiaceae bacterium
MAVRVAINGFGRIGRNVLRAIYESKRTDIEVVAINDLGPVETNAHLLRFDSVHGRFPGEVTVEGDSIDIGNGKIKVTAVRDPATLPWKDLGIDIALECTGIFSAKAKASAHLTAGAKRVLVSAPSDGADATIVYGVNHDKLTKDHLVVSNGSCTTNCLAPIAKVLNDTVGIETGFMTTIHAYTGDQPTLDTMHSDLYRGRAAALSMIPTSTGAAKAIGLVLPELQGKLDGVAIRVPTPNVSVVDLKIVAKKTTTKEEINDAMKRAASQELKGILGTTSHPNVSIDFNHDAHSSTFAFDQTKVQGGTLVRVLSWYDNEWGFSNRMADTAVAMSKLI

SEQ ID NO: 31 - protein, 512 aa, Pseudomonas furukawaii
MSGLLLIAGDCVRRHNVAPFFIPYMWKVLPVTQKPDQCLGEWIDREALAEAMIPMIGQLYRNNNVVTSIYGRGLINRSVIAILKAHRFARHRLVDEAELSVHETFPILKAMSELKLGAASVDLGKMVAKFKAEGNGRSVEQFTRDELADVVGKQNGAAREGTDVVLYGFGRIGRLLARILIEKTGGGDGLRLRAIVVRKGAENDLVKRASLLRRDSVHGPFDGTIVIDEANNTLIANGNLIQVIYSNDPSSIDYTAYGIKNALLVDNTGKWRDAEGLSQHLKCPGIDRVVLTAPGKGALKNIVHGINHGDISADDKIISAASCTTNAIVPVLKAVNDQYGIVNGHVETVHSYTNDQNLIDNFHKGSRRGRSAPLNMVITETGAATAAAKALPVLKGRLTGNAIRVPTPNVSMAILNLNLEKATSREEINEYLRQMAMHSDLHKQIDYVSSQEVVSTDFVGSRHAGVVDAEATICNDNRVVLYVWYDNEFGYSCQVVRVMEDMAGVNPPAFPH

SEQ ID NO: 32 - protein, 444 aa, Bigelowiel lanatans
MALVGAICFSLSGASNVAAPAMRTAPRVASMSRTMPMSMAGQRVSVRPKMIQRREMKAEALSYTVDDIRGMLGDDVANQILAKNTPTGPERNVVLYGFGRIGRLLARIITTIPTNLKLKGIVVRQPKKPDLEKRLELIKRDSVHGPFRGTISMDEADNALIMNGQKVRLIYANQPEEVDYTKYGINDAIVIDNTGVWRDDEGLAKHLSSKGAAQVILTAPGKGVKNIVAGVNSDVIEGSDKILSAASCTTNAVVPALSVLHKKFGIVSGHIETVHSFTNDQNLIDNYHKADRRGRAGPLNMVISETGAGKAVAVALPELAGKLTASAIRVPTPDVSIAVMILDLEKSTTAEEINAAFKSEATGTLDDNLGYSDSKEAVSLDFIGSTHAGEVDALATKCTGNSCIVYVWYDNENGYSNQVVRVVEQWAAKQEAAGLKFEAAEAMA

SEQ ID NO: 33 - protein, 521 aa, Pseudomonas chlororaphis
MIFPDGVWLRSARQARIATQGTRLHNVAPFFSPYMWKVPVTQKPDQCLGEWIDREALAEAMIPLIGQLYRNNNVVSSIYGRSLINQSVIAILKAHRFARHRSSDDSELSVHETFPLLKAMSELKLGAASVDLGKLAFKFRNEGNGRTAEQFVREELADVVGQQNASPRKGTDVVLYGFGRIGRLLARILIEKTGGGDGLRLRAIVVRKGAENDLTKRASLLRRDSVHGSFNGTITIDEANNTITANGNLIQVIYAKNPTEVDYTQYGIKDALLVDNTGVWRDADGLGQHLACPGIDRVVLTAPGKGKLKNIVHGINHADITADDKIVSAASCTTNAIVPVLKAVNDKFGIVNGHVETVHSYTNDQNLIDNFHKGDRRGRSAALNMVITETGAATAAAKALPELAGKLTGNAIRVPTPNVSMAILNLNLEKAATREEMNEYLRYMALHSDLHKQIDFVNSQEVVSTDFVGSRHAGVVDAEATITQDNRVVLYVWYDNEFGYSCQVVRVMEDMAGVNPPAFPR

SEQ ID NO: 34 - protein, 340 aa, Saccharolobus solfataricus
MINVAVNGYGTIGKRVADAIIKQPDMKLVGVAKTSPNYEAFIAHRRGIRIYVPQQSIKKFEESGIPVAGTVEDLIKTSDIVVDTTPNGVGAQYKPIYLQLQRNAIFQGGEKAEVADISFSALCNYNEALGKKYIRVVSCNTTALLRTICTVNKVSKVEKVRATIVRRAADQKEVKKGPINSLVPDPATVPSHHAKDVNSVIRNLDIATMAVIAPTTLMHMHFINITLKDKVEKKDILSVLENTPRIVLISSKYDAEATAELVEVARDLKRDRNDIPEVMIFSDSIYVKDDEVMLMYAVHQESIVVPENIDAIRASMKLMSAEDSMRITNESLGILKGYLI

SEQ ID NO: 35 - protein, 158 aa, Pseudomonas viridiflava
ARKVIISAPASGADATIVYGVNHDTLRQSHQIISSASCTTNCLAPVAQVLHRELGIESGLMTTIHAYTNDQNLIDVYHTDPYRARSATQSMIPSKTGAAEAVGLVLPELAGKLTGMAVRVPVINVSLVDLTVTLKRETTAEEVNALMKEATQHSKVVG

SEQ ID NO: 36 - protein, 356 aa, Kluyveromyces lactis
MPDMTNESSSKPAQINIGINGFGRIGRLVLRAALTHPEVKVRLINNPSTTPEYAAYLFKYDSTHGKYRGEVEFDDERIIIQNDHVSAHIPLSHFREPERIPWASYNVDYVIDSTGVFKEVDTASRHKGVKKVIITAPSKTAPMYVYGVNHVKYNPLTDHVVSNASCTTNCLAPLVKALDDEFGIEEALMTTIHATTASQKTVDGTSSGGKDWRGGRSCQGNIIPSSTGAAKAVGKILPELNGKITGMSIRVPTINISLVDLTFRTAKKTSYDDIMKALEQRSRSDMKGVLGVTKDAVVSSDFTSDSRSSIVDAKAGIELNDHFFKVLSWYDNEYGYSSRVVDLSIFMAQKDFEAGV

SEQ ID NO: 37 - protein, 68 aa, Geobacillus stearothermophilus
MALRVPTPNVSLVDLVVDVKRDVTVEEVNEALLRAANGPLKGILDFTMEPLVSIDFNTNPHEPAFGDH

SEQ ID NO: 38 - protein, 234 aa, Streptococcus pyogenes
LAISPFNYPVNLAGSKIAPALIAGNVVALKPPTQGSISGLLLAEAFAEAGIPAGVFNTITGRGSVIGDYIVEHEAVNFINFTGSTPIGEGIGKLAGMRPIMLELGGKDSAIVLEDADLVLAAKNIVAGAFGYSGQRCTAVKRVLVMDKVADQLAAEIKTLVEKLSVGMPEDDADITPLIDTSAADFVEGLIKDATDKGATALTAFNREGNLISPVLFDHVTTDMRLAWEEPFGP

SEQ ID NO: 39 - protein, 501 aa, Thermoproteus tenax
MRAGLLEGVIKEKGGVPVYPSYLAGEWGGSGQEIEVKSPIDLATIAKVISPSREEVERTLDVLFKRGRWSARDMPGTERLAVLRKAADIIERNLDVFAEVLVMNAGKPKSAAVGEVKAAVDRLRLAELDLKKIGGDYIPGDWTYDTLETEGLVRREPLGVVAAITPFNYPLFDAVNKITYSFIYGNAVVVKPSISDPLPAAMAVKALLDAGFPPDAIALLNLPGKEAEKIVADDRVAAVSFTGSTEVGERVVKVGGVKQYVMELGGGDPAIVLEDADLDLAADKIARGIYSYAGQRCDAIKLVLAERPVYGKLVEEVAKRLSSLRVGDPRDPTVDVGPLISPSAVDEMMAAIEDAVEKGGRVLAGGRRLGPTYVQPTLVEAPADRVKDMVLYKREVFAPVALAVEVKDLDQAIELANGRPYGLDAAVFGRDVVKIRRAVRLLEVGAIYINDMPRHGIGYYPFGGRKKSGVFREGIGYAVEAVTAYKTIVFNYKGKGVWKYE

SEQ ID NO: 40 - protein, 475 aa, Streptococcus mutans
MTKQYKNYVNGEWKLSENEIKIYEPASGAELGSVPAMSTEEVDYVYASAKKAQPAWRSLSYIERAAYLHKVADILMRDKEKIGAVLSKEVAKGYKSAVSEVVRTAEIINYAAEEGLRMEGEVLEGGSFEAASKKKIAVVRREPVGLVLAISPFNYPVNLAGSKIAPALIAGNVIAFKPPTQGSISGLLLAEAFAEAGLPAGVFNTITGRGSEIGDYIVEHQAVNFINFTGSTGIGERIGKMAGMRPIMLELGGKDSAIVLEDADLELTAKNIIAGAFGYSGQRCTAVKRVLVMESVADELVEKIREKVLALTIGNPEDDADITPLIDTKSADYVEGLINDANDKGAAALTEIKREGNLICPILFDKVTTDMRLAWEEPFGPVLPIIRVTSVEEAIEISNKSEYGLQASIFTNDFPRAFGIAEQLEVGTVHINNKTQRGTDNFPFLGAKKSGAGIQGVKYSIEAMTTVKSVVFDIK

SEQ ID NO: 41 - protein, 481 aa, Alkalihalobacillus halodurans
MALQTEQFNANILRNGEWVESRTGERISISAPASGVALGSIPALSQEEVNDAIQGAKDAQKIWKIRPIHERVDLLYAWADLLEERKEIIGELIMHEVAKPKKSAIGEVSRTADIIRHTADEALRLNGETLKGDQFKGGSSKKIALVEREPLGVVLAISPFNYPVNLAAAKIAPALVTGNTVVFKPATQGSLSGIKMVEALADAGAPEGIIQVVTGRGSVIGDHLVEHPGIDMITFTGGTTTGERISEKAKMIPVVLELGGKDPAIVLDDADLKLTASQIVSGAFSYSGQRCTAIKRVFVQDSVADQLVANIKELVEQLTVGSPEDDADITPVIDEKSAAFIQGLIDDALENGATLLSGNKRQGNLLSPTLLDDVTPAMRVAWEEPFGPVLPIIRVKDANEAISLSNQSDYGLQASIFTKDTDRAINIGKHLEVGTVHINAKTERGPDHFPFLGVKKSGLGVQGIKPSLLSMTRERVTVLNL

SEQ ID NO: 42 - protein, 470 aa, Saccharolobus solfataricus
MMSQEVIEVRSPSNLNVIGTVKRMHKDEVRGEIEEAYKGFEIISKMPLYKRTAILRKVSEILEREQERLAKLLAMEAGKPIRDSRVEVLRASRLFRHAAEEVGMVLEGKNYRVDAYEYPPGNENRIVVSTREPIGVVTAILPFNFPINSFAHKVAPALAVGNSVVVKPSINTPLAAIEMKKILVEAGLPDSAVRVVTGYSSEIGDEIITHPLVGLITLTGSTQTGLKIASKAVSLGKRIIMELGGSDPIIILEDANIERASSIAVRARFEYAGQNCNAGKRIIVREEIYDKFVKAFNDKARALKVGDPLDETTDVGPVINKESVENLNSVLADAKAKGGKVEILNKGPESGSFFPLTMVTNPSLDMLVLKSEVFGPIVPIVSVKSDEEAIRIANSTEYGLQSAIFTNDVNRALKLSRELKFGAVIINDSTRLRWDSLAFGGFKKSSIGREGVRETMLEMTENKLIAITLL

SEQ ID NO: 43 - protein, 234 aa, Clostridium acetobutylicum
LAISPFNYPVNLSGSKVAPALIAGNSVVLKPSTTGAISALHLAEIFNAAGLPAGVLNTVTGKGSEIGDYLITHEEVNFINFTGSSAVGKHISKIAGMIPMVLELGGKDAAIVLEDANLETTAKSIVSGAYGYSGQRCTAVKRVLVMDKVADELVELVTKKVKELKVGNPFDDVTITPLIDNKAADYVQTLIDDAIEKGATLIVGNKRKENLMYPTLFDNVTADMRIAWEEPFGP

SEQ ID NO: 44 - protein, 234 aa, Bacillus cereus
LAIAPFNYPVNLFAAKLAPALIMGNAVIFKPATQGAISGIKMVEALHKAGLPKGLVNVATGRGSVIGDYLVEHEGINMVSFTGGTNTGKHLAKKASMIPLVLELGGKDPGIVREDADLQDAANHIVSGAFSYSGQRCTAIKRVLVHENVADELVGLLKEQVAKLSVGSPEQDSTIVPLIDDKSADFVQGLVDDAVEKGATIVIGNKRERNLIYPTLIDHVTTDMRLAWEEPFAP

SEQ ID NO: 45 - protein, 509 aa, Saccharolobus solfataricus
MEKTSVLIKSKELMEIYELKDGVPYFKTYLSGQWISGDEWQDVISPIDLNIIGKIPKLNWNQIDDTLEHIYRKGRWSIRDTPGEKRLDIYKKMASLLDKFKEDFVNVLMINNGKTKSAAEGEVKAAIERLLRADLDVKETRGDYVPGDWSSETLETEAVVRKEPVGVVLSIVPFNYPLFDTVNKIVYTTVIGNAIIIKPPSSTPLPILMLAKVMELASFPKDSFAIITIPGRDMNKVVGDKRIQAISLTGSTETGEEVVRNAGIKQFIMELGGGDPAIVLSDADLAWAAQRIAAGIISYTGQRCDSVKLVLVEEEVYDTLKDLLIKELTKSVKVGDPRDPLTTVGPVIDVKTVDEWEKAIKDAVEKGGKILFGGKRLGPTYIEPVLIEAPKETLKDMYFYNKEVFASAALLIKVKNIDEALEISNSRKYGLDAAIFGKDINKIRKLQRFLEVGAIYINDYPRHGIGYFPFGGRKDSGIGREGIGYTIQYVTAYKSIVYNYKGKGIWEYL

SEQ ID NO: 46 - protein, 506 aa, Thermococcus sibiricus
MDKLVHNKIFDGIYRPGEDGIPEFKAYVAGQWVFGESFSEVKSPIDGRIIARVSRLSRDQVEEALARVYEKGREEIRNYPGEKRVQSFLKAAGLMREAFEDFVNVLILDAGKPKSNAVGETKATIERLEKTTFESRRMLGDYVPGDWSEETLESEGIVKREPYGLILAISPFNYPLFISAAKVVPALLSGNTVLLKPASADPLAAIMFVRVLELAGFPKESFALLTIPGSFMDDVVKDRRIRAITFTGSTEVGEHIIRTGGIKAYHLELGGKDPAIVLDDANLELASEKIVKGMVSFSGQRCDAIRLILAQEKIYEDLKARILEKLKGMEPRNPLEDENAVMGPLIDESSAEYIEEVYKDAVEKGARPLTEFRRKGNYVWPILLEVSKEKIKDLRAFNEDVFGSMALLIKVKDEDEAIEIAKSSRFGLDASVFSECERRARKVARKLEVGSVFINEYPRHGIGYYPFGGMKDSGVGREGIGYSVEQFTTTKTIVHNFKGYGVWEYL

SEQ ID NO: 47 - protein, 491 aa, Escherichia coli
MAVTQTAQACDLVIFGAKGDLARRKLLPSLYQLEKAGQLNPDTRIIGVGRADWDKAAYTKVVREALETFMKETIDEGLWDTLSARLDFCNLDVNDTAAFSRLGAMLDQKNRITINYFAMPPSTFGAICKGLGEAKLNAKPARVVMEKPLGTSLATSQEINDQVGEYFEECQVYRIDHYLGKETVLNLLALRFANSLFVNNWDNRTIDHVEITVAEEVGIEGRWGYFDKAGQMRDMIQNHLLQILCMIAMSPPSDLSADSIRDEKVKVLKSLRRIDRSNVREKTVRGQYTAGFAQGKKVPGYLEEEGANKSSNTETFVAIRVDIDNWRWAGVPFYLRTGKRLPTKCSEVVVYFKTPELNLFKESWQDLPQNKLTIRLQPDEGVDIQVLNKVPGLDHKHNLQITKLDLSYSETFNQTHLADAYERLLLETMRGIQALFVRRDEVEEAWKWVDSITEAWAMDNDAPKPYQAGTWGPVASVAMITRDGRSWNEFE

SEQ ID NO: 48 - protein, 468 aa, Escherichia coli
MSKQQIGVVGMAVMGRNLALNIESRGYTVSIFNRSREKTEEVIAENPGKKLVPYYTVKEFVESLETPRRILLMVKAGAGTDAAIDSLKPYLDKGDIIIDGGNTFFQDTIRRNRELSAEGFNFIGTGVSGGEEGALKGPSIMPGGQKEAYELVAPILTKIAAVAEDGEPCVTYIGADGAGHYVKMVHNGIEYGDMQLIAEAYSLLKGGLNLTNEELAQTFTEWNNGELSSYLIDITKDIFTKKDEDGNYLVDVILDEAANKGTGKWTSQSALDLGEPLSLITESVFARYISSLKDQRVAASKVLSGPQAQPAGDKAEFIEKVRRALYLGKIVSYAQGFSQLRAASEEYNWDLNYGEIAKIFRAGCIIRAQFLQKITDAYAENPQIANLLLAPYFKQIADDYQQALRDVVAYAVQNGIPVPTFSAAVAYYDSYRAAVLPANLIQAQRDYFGAHTYKRIDKEGVFHTEWLD

SEQ ID NO: 49 - protein, 464 aa, Lactobacillus brevis
MKIISIKFVLGGNIMKVTVVGCTHAGTFAIKQILAEHPDAEVTVYERNDVISFLSCGIALYLGGKVADPQGLFYSSPEELQKLGANVQMNHNVLAIDPDQKTVTVEDLTSHAQTTESYDKLVMTSGSWPIVPKIPGIDSDRVKLCKNWAHAQALIEDAKEAKRITVIGAGYIGAELAEAYSTTGHDVTLIDAMDRVMPKYFDADFTDVIEQDYRDHGVQLALSETVESFTDSATGLTIKTDKNSYETDLAILCIGFRPNTDLLKGKVDMAPNGAIITDDYMRSSNPDIFAAGDSAAVHYNPTHQNAYIPLATNAVRQGILVGKNLVKPTVKYMGTQSSSGLALYDRTIVSTGLTLAAAKQQGVNAEQVIVEDNYRPEFMPSTEPVLMSLVFDPDTHRILGGALMSKYDVSQSANTLSVCIQNENTIDDLAMVDMLFQPNFDRPFNYLNILAQAAQAKVAQSVNA

SEQ ID NO: 50 - protein, 450 aa, Levilactobacillus brevis
MKVTVVGCTHAGTFAIKQILAEHPDAEVTVYERNDVISFLSCGIALYLGGKVADPQGLFYSSPEELQKLGANVQMNHNVLAIDPDQKTVTVEDLTSHAQTTESYDKLVMTSGSWPIVPKIPGIDSDRVKLCKNWAHAQALIEDAKEAKRITVIGAGYIGAELAEAYSTTGHDVTLIDAMDRVMPKYFDADFTDVIEQDYRDHGVQLALSETVESFTDSATGLTIKTDKNSYETDLAILCIGFRPNTDLLKGKVDMAPNGAIITDDYMRSSNPDIFAAGDSAAVHYNPTHQNAYIPLATNAVRQGILVGKNLVKPTVKYMGTQSSSGLALYDRTIVSTGLTLAAAKQQGVNAEQVIVEDNYRPEFMPSTEPVLMSLVFDPDTHRILGGALMSKYDVSQSANTLSVCIQNENTIDDLAMVDMLFQPNFDRPFNYLNILAQAAQAKVAQSVNA

SEQ ID NO: 51 - protein, 449 aa, Secundilactobacillus silagincola
MKVAVIGCTHAGTFATKQILAENPDAEVTVYEKNDVISFLSCGIALYLGGQVKDPQGLFYSNPDELSKLGATVLMRHNVTAIDNQAHTITVEDLATHEVTTSSYDKLVMTSGSWPIIPPIPGIDSDKVLLCKNWAHAQTLFQKAENAKRIAVIGAGYIGAELAEAYSLKGHDVTLIDAQDRVMPKYFDSAFTDKIEDEYRAHNVQLALSQKVQGFETTADGVTITTDKEAIDVDLAVLCIGFRPNTDLLAGKVDMAKNGAILTNDYMQTSDPDIFAAGDSAAVHYNPTGKNAYIPLATNAVRQGILVGKNIVKPTVKYMGTQSSSGLALYGTSLCSTGLTYEAAKAQGINAEQVIITDNYRPEFMPTTLPVTMSLVFNPDTREILGGALMSKYDISQSANLLSVCIQNHNTIDDLAMVDMLFQPNFDRPFNYLNILGQAAQEKLTTVKA

SEQ ID NO: 52 - protein, 450 aa, Secundilactobacillus folii
MKVAVIGCTHAGTFATKQILAENPDAEVTVYERNDVISFLSCGIALYLGGEVKDPQGLFYSSPDELSGLGAKVLMRHDVKSIDNQAHTITVEDLDSHETTTTSYDKLVMTTGSWPVIPSIPGIKSDRVLLCKNWAHAQALFNRAKDSKRIAVIGAGYIGAELAEAYSLKNHDVTLIDAQDRVMPKYFDAAFTDKVEDEYTKHGVALALSQKVQSFETTDDAVTIHTDKESVTVDLAILCIGFKPNTILLEGKVDMAANGAILTNDYMQSSDPDIFAAGDSAAVHYNPTGKNAYIPLATNAVRQGILVGKNLVKPTVKYMGTQSTSGLSLYGISLCSTGLTYEAAQEQGINAQQVIITDNYRPEFMPTTLPVTMSLVFNPETREILGGALMSKYDISQSANLLSVCIQNHNTIDDLAMVDMLFQPNFDRPFNYLNILGQAAQQKLAEAVKA

SEQ ID NO: 53 - protein, 451 aa, Lactobacillus kimchicus
MKVVIIGCTHAGTFATKQILTEHPDAEVTVYERNDVISFLSCGIALYLGGQVKDPQGLFYSNPQELAELGATIKMRHDVKSVDNVAHTVVVEDLNTHETTTVLYDKLVMTTGSWPVIPPIPGIDSDRVKLCKNWVHAQTLFKEAPTAKRIAVIGAGYIGAELAEAYSLKGHEVTLIDGEDRVMPKYFDQTFTTKIEANYQNHGVRLALNQRVQSFETTTDAVTIHTDKDAITADLAILCIGFRPNTELVKGKVDMAQNGAILTNDYMQSSDPDIFAAGDAAAVHYNPTGKNAYIPLATNAVRQGILVGKNIAQPTVKYMGTQSSSGLALYGVSICSTGLTYEAAKAQGLAAEQVIVEDNYRPEFMPTTTPVLMSLVFDPNTRQILGGALMSDYDISQSANLLSVCIQNQNTIDDLAMVDMLFQPNFDRPFNYLNILAQAAQEKVAKATAEA

SEQ ID NO: 54 - protein, 445 aa, Lentilactobacillus curieae
MKIAVIGCTHAGTFAIKTVLAEHPDAEVTVYEKNDNISFLSCGIALYLGGEVKDPQGLFYSSPEELSKLGATVKMQHSVESVDPDKKELTVKNLEDNTEFTDTYDKLVVTSGSWPVIPPIPGVDNKNVYLCKNYGHAKALFEKAKDAKRIIVIGAGYIGAELAEAYSKSGHDVTLLDGADRVMPKYFDSEFTDVIEQDFRDHGVTLGLGEKVQEISGDDVATVKTDKNSYEADLVVLCVGFRPNNDLVKGKLDMTDNGALITNDYMQTSNPDIYAAGDSVAVHYNPTHGLAYIPLATNAVRQGILVGKNIETPTVKYIGTQSSSGLKLYDQIMVSTGLTLDSAKRAGINADRVVVKDNYRPEFMPTTAEVLMSLVYDKDSRKILGGSLMSTYDISQSANTLSVCIQDGDTIDELAMVDMLFQPQFDRPFNYLNILAQAAQAKADK

SEQ ID NO: 55 - protein, 452 aa, Lentilactobacillus parakefiri
MKVAVIGCTHAGTFAIKTILAEHPDAEVTVYEKNDNISFLSCGIALYLGGHVKDPQGLFYSSPDELKKLGANVKMNHEVTNVDPASKTIEVSDLKNDQQFEDTYDKLVVTSGSWPVIPPIKGVDNKNVYLCKNWNHAKELFETAPEKKRITIVGAGYIGAELAEAYNTKGHDVTLLDGADRIMSKYFDKEFTDVVEKDFTDHGVTLGLDQQVTEISGEDTVTIKTTKGSYETDLVVLCVGFRPNTGLFKGKLAMTDNGALITNEYMQTSDPDIYGAGDSVAVHYNPTHGLAYIPLATNAVRQGILVGKNIEKPTVKYMGTQSSSGLKLYDKTIVSTGLTMAAAKAQNINASEVIITDNYRPEFMPTTEPVLMSLVYDPDSRRILGGSLLSTYDVSQSANTLSVCIQNENTIDDLAMVDMLFQPQFDRPFNYLNILGQAAQEQADKLTKAASK

SEQ ID NO: 56 - protein, 452 aa, Lactobacillus farraginis
MKIAVIGCTHAGTFAVKTILSEHPDAQVTVYEKNDNISFLSCGIALYLGQQVKDPQGLFYSNPEELKTLGANVKMNHEVTDVDPEAKTITVTNLTEGKQFTDTYDKLVATTGSWPIMPPIQGIDNKNVYLCKNWNHAQELFKTAPAKKRITVVGAGYIGAELAEAYSTTGHDVTLIDGADRVMSKYFDRQFTDVVEQDYKDHGVTLALGQQVTGISGDDTVTITTTKGSYETDLVIMCVGFKPNTSLFKGKLEMLKNGALITNAYMQTSDPDIYAAGDSVAVHYNPTNSPAYIPLATNAVRQGILVGKNIEQPTVKYMGTQSSSGLKLYDRTIVSTGLTMAAANQQGINAAVVTITDNYRPEFMPTTEPVLMSLVYNPENRQILGGTLMSTYDVSQSANTLSVCIQNQNTIDDLAMVDMLFQPQFDRPFNYLNILGQAAQQQLAQQADSVKA

SEQ ID NO: 57 - protein, 414 aa, Staphylococcus aureus
MTENNNLVTSTQGIIKEALHKLGFDEGMYDLIKEPLRMLQVRIPVRMDDGTVKTFTGYRAQHNDAVGPTKGGVRFHPDVDEEEVKALSMWMTLKCGIVNLPYGGGKGGIVCDPRQMSIHEVERLSRGYVRAISQFVGPNKDIPAPDVFTNSQIMAWMMDEYSALDKFNSPGFITGKPIVLGGSHGRDRSTALGVVIAIEQAAKRRNMQIEGAKVVIQGFGNAGSFLAKFLYDLGAKIVGISDAYGALHDPNGLDIDYLLDRRDSFGTVTNLFEETISNKELFELDCDILVPAAISNQITEDNAHDIKASIVVEAANGPTTPEATRILTERGILLVPDVLASAGGVTVSYFEWVQNNQGYYWSEEEVNEKLREKLEAAFDTIYELSQNRKIDMRLAAYIIGIKRTAEAARYRGWA

SEQ ID NO: 58 - protein, 424 aa, Bacillus subtilis
MSAKQVSKDEEKEALNLFLSTQTIIKEALRKLGYPGDMYELMKEPQRMLTVRIPVKMDNGSVKVFTGYRSQHNDAVGPTKGGVRFHPEVNEEEVKALSIWMTLKCGIANLPYGGGKGGIICDPRTMSFGELERLSRGYVRAISQIVGPTKDIPAPDVYTNSQIMAWMMDEYSRLREFDSPGFITGKPLVLGGSQGRETATAQGVTICIEEAVKKKGIKLQNARIIIQGFGNAGSFLAKFMHDAGAKVIGISDANGGLYNPDGLDIPYLLDKRDSFGMVTNLFTDVITNEELLEKDCDILVPAAISNQITAKNAHNIQASIVVEAANGPTTIDATKILNERGVLLVPDILASAGGVTVSYFEWVQNNQGYYWSEEEVAEKLRSVMVSSFETIYQTAATHKVDMRLAAYMTGIRKSAEASRFRGWV

SEQ ID NO: 59 - protein, 1620 aa, Pseudomonas aeruginosa
MAFFTAASKADFQHQLQTALAQHLGDKALPQVTLFAEQFFSLISLDELTQRRLSDLVGCTLSAWRLLERFDRDQPEVRVYNPDYEKHGWQSTHTAVEVLHPDLPFLVDSVRMELNRRGYSIHTLQTNVLSVRRSAKGELKEILPKGSQGKDVSQESLMYLEIDRCAHAGELRALEKAILEVLGEVRVTVADFEPMKAKARELLTWLGKAKLKVPAEELKEVRSYLEWLLDNHFTFLGYEEFSVADEADGGRMVYDEKSFLGLTRLLRAGLSKDDLHIEDYAVAYLREPVLLSFAKAAHPSRVHRPAYPDYVSIRELDGKGRVIRECRFMGLFTSSVYNESVNDIPFIRGKVAEVMRRSGFDTKAHLGKELAQVLEVLPRDDLFQTPVDELFSTALAIVRIQERNKIRVFLRKDPYGRFCYCLAYVPRDVYSTETRLKIQQVLMERLQASDCEFWTFFSESVLARVQFILRVDPKSRIDIDPARLEEEVIQACRSWQDDYSSLVVENLGEAKGTNVLADFPKGFPAGYRERFAPHFAVVDLQHLLSLSEQRPLVMSFYQPLAQGEQQLHCKLYHADTPLALSDVLPILENLGLRVLGEFPYRLRHQNGREYWIHDFAFTYAEGLDVDIQQLNEILQDAFVHIVSGDAENDAFNRLVLTANLPWRDVALLRAYARYLKQIRLGFDLGYIASALNAHTDIARELVRLFKTRFYLARKLTAEDLEDKQQKLEQAILGALDEVQVLNEDRILRRYLDLIKATLRTNFYQPDGNGQNKSYFSFKFNPKAIPELPRPVPKYEIFVYSPRVEGVHLRGGKVARGGLRWSDREEDFRTEVLGLVKAQQVKNAVIVPVGAKGGFVPRRLPLGGSRDEIQAEAIACYRIFISGLLDITDNLKEGEVVPPANVVRHDEDDPYLVVAADKGTATFSDIANGIAAEYGFWLGDAFASGGSAGYDHKGMGITAKGAWVSVQRHFRERGIDVQKDNISVIGIGDMAGDVFGNGLLMSDKLQLVAAFNHMHIFIDPNPDAASSFVERQRLFNLPRSSWADYDAKLISAGGGIFLRSAKSIAITPEMKARFDIQADRLAPTELIHALLKAPVDLLWNGGIGTYVKSSKETHADVGDKANDGLRVDGRELRAKVVGEGGNLGMTQLARVEFGLHGGANNTDFIDNAGGVDCSDHEVNIKILLNEVVQAGDMTEKQRNALLVKMTDAVGALVLGNNYKQTQALSLAQRRARERIAEYKRLMGDLEARGKLDRALEFLPSDEELAERISAGQGLTRAELSVLISYSKIDLKESLLKSLVPDDDYLTRDMETAFPALLAEKFGDAMRRHRLKREIVSTQIANDLVNHMGITFVQRLKESTGMSAANVAGAYVIVRDVFHLPHWFRQIENLDYQVPADIQLTLMDELMRLGRRATRWFLRSRRNELDAARDVAHFGPRIAALGLKLNELLEGPTRELWQARYQTYVDAGVPELLARMVAGTSHLYTLLPIIEASDVTGQDTAEVAKAYFAVGSALDLTWYLQQITNLPVENNWQALAREAFRDDLDWQQRAITVSVLQMQDGPKEVEARVGLWLEQHLPLVERWRAMLVELRAASGTDYAMYAVANRELMDLAQSSQHGVCIP

SEQ ID NO: 60 - protein, 450 aa, Clostridium symbiosum
MSKYVDRVIAEVEKKYADEPEFVQTVEEVLSSLGPVVDAHPEYEEVALLERMVIPERVIEFRVPWEDDNGKVHVNTGYRVQFNGAIGPYKGGLRFAPSVNLSIMKFLGFEQAFKDSLTTLPMGGAKGGSDFDPNGKSDREVMRFCQAFMTELYRHIGPDIDVPAGDLGVGAREIGYMYGQYRKIVGGFYNGVLTGKARSFGGSLVRPEATGYGSVYYVEAVMKHENDTLVGKTVALAGFGNVAWGAAKKLAELGAKAVTLSGPDGYIYDPEGITTEEKINYMLEMRASGRNKVQDYADKFGVQFFPGEKPWGQKVDIIMPCATQNDVDLEQAKKIVANNVKYYIEVANMPTTNEALRFLMQQPNMVVAPSKAVNAGGVLVSGFEMSQNSERLSWTAEEVDSKLHQVMTDIHDGSAAAAERYGLGYNLVAGANIVGFQKIADAMMAQGIAW

SEQ ID NO: 61 - protein, 1092 aa, Saccharomyces cerevisiae
MLFDNKNRGALNSLNTPDIASLSISSMSDYHVFDFPGKDLQREEVIDLLDQQGFIPDDLIEQEVDWFYNSLGIDDLFFSRESPQLISNIIHSLYASKLDFFAKSKFNGIQPRLFSIKNKIITNDNHAIFMESNTGVSISDSQQKNFKFASDAVGNDTLEHGKDTIKKNRIEMDDSCPPYELDSEIDDLFLDNKSQKNCRLVSFWAPESELKLTFVYESVYPNDDPAGVDISSQDLLKGDIESISDKTMYKVSSNENKKLYGLLLKLVKEREGPVIKTTRSVENKDEIRLLVAYKRFTTKRYYSALNSLFHYYKLKPSKFYLESFNVKDDDIIIFSVYLNENQQLEDVLLHDVEAALKQVEREASLLYAIPNNSFHEVYQRRQFSPKEAIYAHIGAIFINHFVNRLGSDYQNLLSQITIKRNDTTLLEIVENLKRKLRNETLTQQTIINIMSKHYTIISKLYKNFAQIHYYHNSTKDMEKTLSFQRLEKVEPFKNDQEFEAYLNKFIPNDSPDLLILKTLNIFNKSILKTNFFITRKVAISFRLDPSLVMTKFEYPETPYGIFFVVGNTFKGFHIRFRDIARGGIRIVCSRNQDIYDLNSKNVIDENYQLASTQQRKNKDIPEGGSKGVILLNPGLVEHDQTFVAFSQYVDAMIDILINDPLKENYVNLLPKEEILFFGPDEGTAGFVDWATNHARVRNCPWWKSFLTGKSPSLGGIPHDEYGMTSLGVRAYVNKIYETLNLTNSTVYKFQTGGPDGDLGSNEILLSSPNECYLAILDGSGVLCDPKGLDKDELCRLAHERKMISDFDTSKLSNNGFFVSVDAMDIMLPNGTIVANGTTFRNTFHTQIFKFVDHVDIFVPCGGRPNSITLNNLHYFVDEKTGKCKIPYIVEGANLFITQPAKNALEEHGCILFKDASANKGGVTSSSMEVLASLALNDNDFVHKFIGDVSGERSALYKSYVVEVQSRIQKNAELEFGQLWNLNQLNGTHISEISNQLSFTINKLNDDLVASQELWLNDLKLRNYLLLDKIIPKILIDVAGPQSVLENIPESYLKVLLSSYLSSTFVYQNGIDVNIGKFLEFIGGLKREAEASA

SEQ ID NO: 62 - protein, 420 aa, Pyrococcus furiosus
MVEQDPYEIVIKQLERAAQYMEISEEALEFLKRPQRIVEVTIPVEMDDGSVKVFTGFRVQHNWARGPTKGGIRWHPEETLSTVKALAAWMTWKTAVMDLPYGGGKGGIIVDPKKLSDREKERLARGYIRAIYDVISPYEDIPAPDVYTNPQIMAWMMDEYETISRRKTPAFGIITGKPLSIGGSLGRIEATARGASYTIREAAKVLGWDTLKGKTIAIQGYGNAGYYLAKIMSEDFGMKVVAVSDSKGGIYNPDGLNADEVLKWKNEHGSVKDFPGATNITNEELLELEVDVLAPAAIEEVITKKNADNIKAKIVAEVANGPVTPEADEILFEKGILQIPDFLCNAGGVTVSYFEWVQNITGYYWTIEEVRERLDKKMTKAFYDVYNIAKEKNIHMRDAAYVVAVQRVYQAMLDRGWVKH

SEQ ID NO: 63 - protein, 421 aa, Thermococcus kodakarensis
MVEIDPFEMAVQQLERAAQFMDISEEALEWLKRPMRIVEVSVPVEMDDGSVKVFTGFRVQHNWARGPTKGGIRWHPAETLSTVKALATWMTWKVAVVDLPYGGGKGGIIVDPKKLSEREQERLARSYIRAVYDVIGPWTDIPAPDVYTNPKIMAWMMDEYETIMRRKGPAFGVITGKPPGVGGIVARMDATARGAAFTIREAAKALGWDDLKGKTIAIQGYGNAGYYLHKIMSEEFGMKVVAVSDSKGGIYNPDGLPPADEVLKWKKEHGSVKDMPGTQNITNEELLELEVDILAPSAIEGVITKENADNVKAKIVAEVANGPVTPEADEILHEKGILQIPDFLCNAGGVTVSYFEWVQNINGFYWTVEETRKRLDDKMTKAFWDVFNTHKEKNIHMRDAAYVVAVSRVYEAMKHRGWVKK

SEQ ID NO: 64 - protein, 448 aa, Lactobacillus sp.
MSNAKSYIESVKDTIQRRDPNQTEFQEAAFTVLDTLEPVLDKHPEYIKANLLERLVEPERAIQFRVPWQSDDGQFHVNRGFRVQFNSAIGPYKGGLRLHPSVSLDVVKFLGFEQVLKNSLTGLPIGGAKGGSDFNPKGKSDSEVMRFCQSFMTELQRHIGPDLDVPAGDIGVGGREIGFLFGAYKRLNGYQNGVLTGKGLTFGGSLARPEATGFGLLYYTHAMLKALGDTLTNKKIKVSGAGNVATYAIQKATEYGATVITCSDSDGYVYDPNGIDFKLVKQIKEVERGRIKEYAQRVETAEYHDGSVWDFDINYDIALPCATQNEISKDQAKLIAKDRVKYVCEGANMPSVPDAIATYRDNNIYYGPAKAANAGGVAVSALEMSQNSERLSWSFETVDNRLHDIMDNIFEISEAADKEYQLNQDYQAGANIAGFEKVAQTMMAQGLV

SEQ ID NO: 65 - protein, 1646 aa, Streptomyces sp.
MQTKLDEAKAELLERAARVAENSPVGGHLPTGTADEGTPDRESVLAFLQRYYLHTAPEDLTDRDPVDVFGAALSHYRLAENRPQGTANVRVLTPTVEENGWACSHSIVEVVTDDMPFLVDSVTNELTRQGRGIHVVIHPQVVVRRDLTGQLIEVLPQRPSGDRSALPHDAHTESWIHVEIDRETDRADLKQITADLLRVLSDVREAVEDWEKMREAALRIAEDLPAEPTASDLPEQEVEEARELLRWLAADHFTFLGYREYQLREDDSLAAVPGTGLGILRSDPHHASEDSHPVSPSFERLPADARAKAREHRLLVLTKANSRATVHRPSYLDYIGVKKFDADGNVVGERRFLGLFSSAAYTESVRRVPVIRRKVAEVLQRAGFSPNSHDGRDLLQILETYPRDELFQTPADELQSIVTSVLYLQERRRLRLYLRQDEYGRYYSALVYLPRDRYTTGVRLRIIDILKEELGGISVDFTAWNTESILSRLHFVVRVPQGTELPELSDSDKERIEARLVEAARSWADGFAEALTGELGEERAAELLRRYGNAFPEGYKADHTPRAAVADLCHLEQLTEDSNFALSLYEPVGAAPEERRFKIYRKGEAVSLSAVLPVLSRLGVEVTDERPYELRCADRTASWIYDFGLRMPKAQNGAGDYLGDDGRERFQEAFAATWTGKAENDGFNALVLSAGLTWRQAMVLRAYAKYLRQAGSTFSQDYMEDTLRNNVHTTRLLVSLFEARMSPDRQRAGHEIVDALLEEVDAALDQVASLDEDRILRSFLTVIKATLRTNFFQEALGGQPHDYVSMKFDPQAIPDLPAPRPAFEIWVYSPRVEGVHLRFGKVARGGLRWSDRREDFRTEILGLVKAQMVKNTVIVPVGAKGGFVAKQLPDPGVDREAWLAEGIASYKVFISALLDITDNMVAGEVVPPADVVRHDEDDTYLVVAADKGTATFSDIANGVAESYNFWLGDAFASGGSAGYDHKGMGITARGAWESVKRHFRELDVDTQTEDFTVVGIGDMSGDVFGNGMLLSEHIRLVAAFDHRHIFIDPTPDAATSYAERRRVFELPRSSWADYNTELISAGGGVFPRSAKAIQLNAHIREALGIEGKVSKMTPADLMKAILKAPVDLLWNGGIGTYVKSSAESHGDVGDKANDPIRVDGADLRVKVVGEGGNLGLTQLGRIEFALHGGKINTDAIDNSAGVDTSDHEVNIKILLNGLVTEGDMTVKQRNKLLAEMTDEVGHLVLRNNYAQNTAIANALAQSKDMLHAQQRFMRHLVREGHLDRALEFLPTDRQIRERLTNGQGLTGPETAVLLAYTKITVSDELLHTSLPDDPYLRTLLDAYFPSALRERFPEQMHGHPLAREIITTVLVNDTVNTGGTTYLHRLREETGASLEEIVRAQTAARAIFRSAAVWDGVESLDNRVEAAVQTRIRLHSRRLVERGTRWLLNNRPQPLQLAETVEFFGERVEQVWSQLPKLLKGADLEWYQQIYDELSGAGVPDELATRVAGFSSAFPTLDIVSVADRNGKEPLDVAEVYYDLADRLHITQLMDRIIELPRADRWQSMARASIREDLYAAHAAVTADVLAVGNGTSTPEQRFKAWEEKNAAILGRARSTLEEIQGSETFDLANLSVAMRTMRTLLRTHS

SEQ ID NO: 66 - protein, 1626 aa, Brevibacterium sp.
MTQVSISDVAEEWRKLRGQGAPENFLDAYYPRFETGASDPEAYAAAAARHFAVGTEYDGQTPAIAIYNPAVDSPEFRDNHTVVALVLADMPHLVSSIVSDLAAAGRAIRRVHHPIITVSGSGSDLSIVSPAEAPSVTADTASLPQVSAAPDAQSSDDPQQQSWIRLEIDRIPEDDFAELEQRLREVLAYVAAAARDAQAMASKAKDIAAELSAHAPRPELASEAEAAAALLNWLDGHFTFLGYREYDYTHDAEHSSLEPIEDTSMGISALRPLVKSPLSRAVAEKALEPHVLVLTKANSRSRVIRSSFMDYIGVKTFDSSGEIVGERRFVGVFKPEFYNDSVLNIPVIDRKVSRILSASGFPAGSHSANELLGVLETYPREDLLHEDTEEIFDVVMEIVDMQERRESRVFVRADPYQRFVSVILYLPRDLYNTDARVRVQEVLREFYNADSVDFDVLLSESALARIHFVARMARDEQLPQISAEDVERRIVGAVRSWSEDVHAFLIPAEHGDSGTTNARADLWSKAFPPSYGEHHTPAEAVADVGRFEALDAGHGSAVRLYRPERSADASVRLVLYRYERVGLSEVLPFLTAFGATVIDERPHELDLADGSHRYIYDFGLSFSEELGDDDCERISDAFMAGWEGKKEAGVFDRLIVTGLSWQHVTIIRALGKYLRQAGFTYSDAYVGEVYSDHPEISKLLVDYFAAKFDPAADDESREGAMAEIDDAIEAALSEVASLDADRVLRSSLELLRATLRTNYYIDESGELPTALVLKIRPTELSFVPKPKPALEMWVYSPQVEGAHLRFGAVARGGLRWSDRRDDFRTEVLGLVKAQMVKNALIVPTGAKGGFFPKQLPPMSDRDAWMAAGQAAYEVFIESMLEVSDDLTYGADDSSVVVHPDRVVRHDGDDYYLVVAADKGTARFSDVANAIAERRGFWLGDAFASGGSVGYDHKKMAITSRGAWKSVERHFRELGLNTAADDFTVVGIGDMSGDVFGNGMLRSEHIRLVAAFDHRDIFLDPNPDAARSFVERQRLFDLPRSSWQDYDRELISAGGGVFSRSAKSIDLSAEAASVLGVDPGRRSPADLMSEILKAPVDLVYNGGIGTYIKASTESHAEVGDKSNDSIRIDGADVRARVIGEGGNLGVTQLGRVEAALNGVEINTDAVDNSAGVDSSDHEVNIKLLLRTLVHKGAFAAEEREQVLHSFTDQVADRVLSNNYSQNVVLGEARAQTEAMSGTYGRLLNYLEKHADLDRAVEFLPNADELAKRRFGYYVSPELAVLLAYVKMHAADEILDSVVPDEMWMRRELVSYFPESLQEKYGELIPEHPLHREIATAKLVNRLVDRGGLTYIYRMLEETPASVPQIARVFVVVSEIFGLDDYFEAVCALDNEVPTEVQVKLQHDYVRLLDRSSRWLVQQAPDTLDVDSGIEMYGKVVEALRSRVPELVDGYDAESMQAKAQEYIDDGVPSDLAWRAAALLDEFVLLDITQLAARANESAEDVAEVYYAVNEHFAGSQVLTLIGGLDRSDRWSALARGSMRDDFYSAILSVAETVLAATDSPISGTPDERAKQRVAEWLERNETVVSRVIETTETILGLDSVTQAPLSVLLRSLRSMVRSSTWESEHNS

SEQ ID NO: 67 - DNA, 26 bp, synthetic construct
  source 1..26: mol_type = other DNA
ggcccatggcaaaggtatcgctggag

SEQ ID NO: 68 - DNA, 33 bp, synthetic construct
  source 1..33: mol_type = other DNA
attgcggccgcttagtacagcagacgggcgcga

SEQ ID NO: 69 - DNA, 27 bp, synthetic construct
  source 1..27: mol_type = other DNA
ggcccatggctaacattacctggtgcg

SEQ ID NO: 70 - DNA, 33 bp, synthetic construct
  source 1..33: mol_type = other DNA
gccaagcttttattacttctgattcaggctgcc

SEQ ID NO: 71 - DNA, 35 bp, synthetic construct
  source 1..35: mol_type = other DNA
gccttaattaattattacttctgattcaggctgcc

SEQ ID NO: 72 - DNA, 38 bp, synthetic construct
  source 1..38: mol_type = other DNA
ggccatatgatggctcaaatcttcaattttagttctgg

SEQ ID NO: 73 - DNA, 36 bp, synthetic construct
  source 1..36: mol_type = other DNA
gccttaattaatcattaaccgtgacggcgttcgaac

SEQ ID NO: 74 - DNA, 27 bp, synthetic construct
  source 1..27: mol_type = other DNA
ccgcatatgtcgcgaaaagatggggtg

SEQ ID NO: 75 - DNA, 54 bp, synthetic construct
  source 1..54: mol_type = other DNA
gccttaattaatgatgatgatgatgatgacttcccacctttaccgctttacgcc

SEQ ID NO: 76 - DNA, 26 bp, synthetic construct
  source 1..26: mol_type = other DNA
ccgccatggcgcgaaaagatggggtg

SEQ ID NO: 77 - DNA, 30 bp, synthetic construct
  source 1..30: mol_type = other DNA
cgagcagggcgtcgctatcgccgcgcaata

SEQ ID NO: 78 - DNA, 30 bp, synthetic construct
  source 1..30: mol_type = other DNA
tattgcgcggcgatagcgacgccctgctcg

SEQ ID NO: 79 - DNA, 33 bp, synthetic construct
  source 1..33: mol_type = other DNA
ctgatcacatcgctgaagctcgtccgggcgtgc

SEQ ID NO: 80 - DNA, 34 bp, synthetic construct
  source 1..34: mol_type = other DNA
gcacgcccggacgagcttcagcgatgtgcatcag

SEQ ID NO: 81 - DNA, 32 bp, synthetic construct
  source 1..32: mol_type = other DNA
caaggtattattctgtcatggcggtggtcgcg

SEQ ID NO: 82 - DNA, 32 bp, synthetic construct
  source 1..32: mol_type = other DNA
cgcgaccaccgccatgacagaataataccttg

SEQ ID NO: 83 - DNA, 73 bp, synthetic construct
  source 1..73: mol_type = other DNA
gtaattttacaggcaaccttttattcactaacaaatagctggtggaatataaaatgagacgttgatcggcacg

SEQ ID NO: 84 - DNA, 76 bp, synthetic construct
  source 1..76: mol_type = other DNA
aaaaagagcgaccgaagtcgctctttttagatcacagtgtcatctcaacaccaaagggaaaactgtccatatgcac

SEQ ID NO: 85 - DNA, 73 bp, synthetic construct
  source 1..73: mol_type = other DNA
gtaattttacaggcaaccttttattcactaacaaatagctggtggaatatatggcaaagatagctattaatgg

SEQ ID NO: 86 - DNA, 76 bp, synthetic construct
  source 1..76: mol_type = other DNA
aaaaagagcgaccgaagtcgctctttttagatcacagtgtcatctcaacctattttgctatttttgcaaagtaagc

SEQ ID NO: 87 - DNA, 22 bp, synthetic construct
  source 1..22: mol_type = other DNA
  modified_base 7: mod_base = OTHER; note = Uracil
ataagctgaggtcgcctcagcg

SEQ ID NO: 88 - DNA, 43 bp, synthetic construct
  source 1..43: mol_type = other DNA
  modified_base 10: mod_base = OTHER; note = Uracil
aaactggtctccttcttaaagttaaacaaaattatttctagag

SEQ ID NO: 89 - DNA, 26 bp, synthetic construct
  source 1..26: mol_type = other DNA
  modified_base 10: mod_base = OTHER; note = Uracil
agaccagtttatggcaaaggtatcgc

SEQ ID NO: 90 - DNA, 82 bp, synthetic construct
  source 1..82: mol_type = other DNA
  modified_base 9: mod_base = OTHER; note = Uracil
attccgggtaccattcgcgcccgtctgctgtactgataaaagaaggagaccagtttatggctcaaatcttcaattttagttc

SEQ ID NO: 91 - DNA, 45 bp, synthetic construct
  source 1..45: mol_type = other DNA
  modified_base 9: mod_base = OTHER; note = Uracil
atcaaccgtgacggcgttcgaactcaaccatgaagtctgtcagcg

SEQ ID NO: 92 - DNA, 45 bp, synthetic construct
  source 1..45: mol_type = other DNA
  modified_base 9: mod_base = OTHER; note = Uracil
acggttgataaaagaaggagaccagtttatgcctaacattacctg

SEQ ID NO: 93 - DNA, 25 bp, synthetic construct
  source 1..25: mol_type = other DNA
  modified_base 7: mod_base = OTHER; note = Uracil
agcttattacttctgattcaggctg

SEQ ID NO: 94 - DNA, 24 bp, synthetic construct
  source 1..24: mol_type = other DNA
  modified_base 7: mod_base = OTHER; note = Uracil
atcacttctgattcaggctgcctg

SEQ ID NO: 95 - DNA, 18 bp, synthetic construct
  source 1..18: mol_type = other DNA
  modified_base 8: mod_base = OTHER; note = Uracil
actgaggtcgcctcagcg

SEQ ID NO: 96 - DNA, 49 bp, synthetic construct
  source 1..49: mol_type = other DNA
  modified_base 7: mod_base = OTHER; note = Uracil
aagtgataaaagaaggagaccagtttatggaagtcacagttgttggttg

SEQ ID NO: 97 - DNA, 32 bp, synthetic construct
  source 1..32: mol_type = other DNA
  modified_base 8: mod_base = OTHER; note = Uracil
acctcagtttattaagcgttaactgattgggc

SEQ ID NO: 98 - DNA, 27 bp, synthetic construct
  source 1..27: mol_type = other DNA
  modified_base 7: mod_base = OTHER; note = Uracil
aggttatttagacccatccgcggaaac

SEQ ID NO: 99 - DNA, 34 bp, synthetic construct
  source 1..34: mol_type = other DNA
  modified_base 13: mod_base = OTHER; note = Uracil
aggagatatacatatgtcagcaaagcaagtctcg

SEQ ID NO: 100 - DNA, 23 bp, synthetic construct
  source 1..23: mol_type = other DNA
  modified_base 7: mod_base = OTHER; note = Uracil
ataacctaggctgctgccaccgc

SEQ ID NO: 101 - DNA, 38 bp, synthetic construct
  source 1..38: mol_type = other DNA
  modified_base 13: mod_base = OTHER; note = Uracil
atgtatatctccttcttatacttaactaatatactaag

SEQ ID NO: 102 - DNA, 24 bp, synthetic construct
  source 1..24: mol_type = other DNA
  modified_base 8: mod_base = OTHER; note = Uracil
agccgcataatgcttaagtcgaac

SEQ ID NO: 103 - DNA, 46 bp, synthetic construct
  source 1..46: mol_type = other DNA
  modified_base 9: mod_base = OTHER; note = Uracil
atggtatatctccttattaaagttaaacaaaattatttctacaggg

SEQ ID NO: 104 - DNA, 26 bp, synthetic construct
  source 1..26: mol_type = other DNA
  modified_base 9: mod_base = OTHER; note = Uracil
atataccatatgagccagaatggccg

SEQ ID NO: 105 - DNA, 30 bp, synthetic construct
  source 1..30: mol_type = other DNA
  modified_base 8: mod_base = OTHER; note = Uracil
atgcggctttatcaaccaagcttgcgagcc

SEQ ID NO: 106 - DNA, 30 bp, synthetic construct
  source 1..30: mol_type = other DNA
  modified_base 10: mod_base = OTHER; note = Uracil
aaactggtctccttcttaaagttaaacaaa

SEQ ID NO: 107 - DNA, 46 bp, synthetic construct
  source 1..46: mol_type = other DNA
  modified_base 9: mod_base = OTHER; note = Uracil
agcttggttgataaaagaaggagaccagtttatggctcaaatcttc

SEQ ID NO: 108 - DNA, 30 bp, synthetic construct
  source 1..30: mol_type = other DNA
  modified_base 10: mod_base = OTHER; note = Uracil
agaccagtttatgagccagaatggccgtcc

SEQ ID NO: 109 - DNA, 35 bp, synthetic construct
  source 1..35: mol_type = other DNA
  modified_base 9: mod_base = OTHER; note = Uracil
aaccaagctttattagtcaagatcaacctggaagg

SEQ ID NO: 110 - DNA, 24 bp, synthetic construct
  source 1..24: mol_type = other DNA
ctgctggcatcgccatcgaggctg

SEQ ID NO: 111 - DNA, 24 bp, synthetic construct
  source 1..24: mol_type = other DNA
cagcctcgatggcgatgccagcag

SEQ ID NO: 112 - DNA, 25 bp, synthetic construct
  source 1..25: mol_type = other DNA
ctcttcctgcaggccactgacgctc

SEQ ID NO: 113 - DNA, 25 bp, synthetic construct
  source 1..25: mol_type = other DNA
gagcgtcagtggcctgcaggaagag

SEQ ID NO: 114 - DNA, 1498 bp, Lactobacillus brevis
  source 1..1498: mol_type = other DNA
ccgtcacgctgacctgatgggggtattctgcatcctctcaggcagcctgaatcagaagtgataaaagaaggagaccagtttatgaaagtcaccgtcgttggctgcactcatgctggcacctttgcaattaaacagatactggcggagcatccggatgcagaggtaacagtatatgaacggaacgacgttatttctttcttaagttgcggcattgctttgtatctggggggtaaagtagcggacccccagggtttattttacagtagccctgaagagctgcagaagctgggtgcgaatgtgcagatgaaccacaacgtactggcgatagatccggatcagaagacagtgaccgtagaagatctgacgagccacgcccagacaacagaatcttatgataaactcgtgatgacttcaggctcttggcccatcgtccctaaaattccaggtatcgatagcgatcgcgtcaagttatgtaaaaactgggctcacgcgcaggcccttatagaggacgcgaaggaagcaaaacgcataaccgtgataggtgccggatacataggtgcggagcttgccgaagcatattccaccaccggacacgatgtaacgttaattgatgcaatggaccgcgttatgccaaaatattttgatgcggatttcacggacgttattgaacaggattaccgtgatcacggcgtgcagcttgccctgtctgagacagtggagtcctttactgatagtgctactggtctgactattaaaaccgataagaattcgtatgaaaccgatttggccatactctgtattggcttccggcccaacactgatttgctgaaaggtaaggtagatatggcgccgaacggagcgattatcaccgacgattatatgcgttcttccaatccagatatattcgctgcgggcgattcggctgcagtccactacaatccaacacatcaaaacgcatatatacccttggcaaccaatgcagtacgccaaggaattttggtgggtaaaaatctggtgaaacccacagtaaagtatatgggtacccagtcgtcgagcgggctggccctttatgatcgtaccattgtgagcaccggcttaactcttgcggctgctaaacagcagggtgtgaacgctgaacaggtgattgtagaggacaactatcggccggaatttatgccgtctacagaacctgtgctgatgagcctggtcttcgatccagacacccatcgtattcttggtggagctctgatgtccaaatatgatgtgagtcaatctgcaaatactctttctgtttgtattcagaatgaaaatacgatcgacgacctggctatggtagatatgttatttcagccgaactttgatcggccattcaattaccttaatattctggcccaggcggcgcaggcaaaagtagcacagtcggtaaatgcgtaagctgaggtcgcctcagcggccggcccctgcaggcgccggcgactagtcttggactcctgttgat

SEQ ID NO: 115 - DNA, 1498 bp, Secundilactobacillus folii
  source 1..1498: mol_type = other DNA
ccgtcacgctgacctgatgggggtattctgcatcctctcaggcagcctgaatcagaagtgataaaagaaggagaccagtttatgaaagtggccgtgattggctgtactcatgccggtacatttgcgacgaaacagattttggcagaaaatccagacgcggaagtaacagtttatgaacgcaatgacgtcatttcgtttttgagttgcggcattgcactttatctgggtggagaagtgaaggatccccagggcctgttttattcttcgccggatgaactgagcggacttggcgctaaagttcttatgcggcacgatgtcaaatccatcgacaatcaagcccacacaattacggtggaagatctggactctcatgagacaacgactacgagctatgataaactggttatgaccactgggagttggccggtgattccgtcaattccgggtatcaaatctgatcgtgtattactgtgcaaaaactgggcccacgcgcaagctctctttaatcgcgctaaagattctaaacgtattgctgtgatcggagcggggtatattggcgctgagttagccgaagcatactcgcttaagaatcacgatgtgacccttattgacgcccaggaccgggtgatgccgaaatattttgatgcagctttcacagataaagttgaggacgagtatacaaaacatggggttgccttggctttgagtcagaaagtccagagctttgaaacaacagacgatgcagtaacgatacacactgataaagagagtgttactgtggatttagccattctttgcattggtttcaaacctaacacaattcttcttgaaggcaaagtggatatggcagccaatggtgccattctcaccaatgactatatgcaaagctctgatcctgatatatttgcggcaggagattctgcggcggttcactataacccaacaggcaagaacgcatatattccgttagcaaccaatgcagtccgacagggcatcttagttggtaaaaatttggtaaaacccaccgtcaaatatatgggaacgcagtcgacctctggccttagcctgtacggcatttcattatgcagcactgggttaacgtatgaagccgcacaggagcagggtattaatgcgcagcaagtcattattacagataattatcgcccagaattcatgcctacgacattaccggtcaccatgagcctggttttcaaccctgagacgcgtgagatactcggtggtgccctgatgagtaaatacgatatatcccagagtgcgaacctgctttctgtgtgtatacaaaatcacaatactatcgatgatctggcgatggtggatatgttattccagccgaattttgatcggccttttaattacctcaatatactgggccaggctgctcaacagaagctggccgaagcagtcaaagcataagctgaggtcgcctcagcggccggcccctgcaggcgccggcgactagtcttggactcctgttgat

SEQ ID NO: 116 - DNA, 1504 bp, Lentilactobacillus parakefiri
  source 1..1504: mol_type = other DNA
ccgtcacgctgacctgatgggggtattctgcatcctctcaggcagcctgaatcagaagtgataaaagaaggagaccagtttatgaaagttgcggtaattggatgcacccacgcgggtacttttgccattaaaactattcttgctgagcatccagatgcggaggtaacggtgtatgaaaaaaatgacaatatttcgtttctgtcgtgtgggatcgcgttgtaccttggcggtcacgtcaaggacccgcagggactgttttattcatcgccggatgagctgaaaaaattgggagcgaacgttaaaatgaatcacgaagtcaccaatgttgacccggcctcgaagacgattgaagtaagtgacttgaaaaacgaccagcagtttgaagatacatacgataaactggttgttacctcaggcagttggcccgtgatccctccgatcaaaggcgtcgataacaaaaacgtctacctgtgtaaaaattggaatcatgccaaagagctttttgaaacggctcccgaaaaaaagcgtataacaatcgttggtgcgggatatataggtgcggaactggccgaggcgtacaatactaagggtcacgatgtcacacttctggacggcgcagatcgtattatgtcaaaatactttgataaagaatttactgatgtggtggagaaagattttaccgatcacggagtgaccttaggcttagatcagcaagtgactgagatttcaggcgaagacacggtgactattaaaacgacgaaaggctcctatgagacagatcttgtcgtcctgtgcgtaggtttccgacctaacaccggccttttcaagggtaagctggctatgacggataacggggcattaattaccaacgaatacatgcagaccagcgatccagacatatatggagccggtgactcagtggctgttcactataatccgacccatggactggcttacattccgttagcgacaaacgccgtacgtcagggtattcttgtgggcaaaaacatagaaaagcctactgttaaatacatgggcacacagtcatcaagtggtctgaaactgtatgacaaaacaatcgtcagtactggtcttacaatggccgctgctaaagctcagaacattaatgctagtgaagtaattattacagacaactatcgcccggaatttatgccgacgacagaacctgttcttatgtccctcgtgtatgacccggatagccggcgcatcttgggaggaagcctcctctctacgtatgatgttagtcagagcgccaacactctgtccgtatgtatacagaatgagaatactatcgacgatttggccatggttgatatgctgtttcagccgcagttcgaccgtccgtttaattacctgaatattctgggtcaggccgcgcaggaacaggcggataaattaaccaaagccgctagtaaataagctgaggtcgcctcagcggccggcccctgcaggcgccggcgactagtcttggactcctgttgat

SEQ ID NO: 117 - protein, 362 aa, Escherichia coli
MAQIFNFSSGPAMLPAEVLKQAQQELRDWNGLGTSVMEVSHRGKEFIQVAEEAEKDFRDLLNVPSNYKVLFCHGGGRGQFAAVPLNILGDKTTADYVDAGYWAASAIKEAKKYCTPNVFDAKVTVDGLRAVKPMREWQLSDNAAYMHYCPNETIDGIAIDETPDFGADVVVAADFSSTILSRPIDVSRYGVIYAGAQKNIGPAGLTIVIVREDLLGKANIACPSILDYSILNDNGSMFNTPPTFAWYLSGLVFKWLKANGGVAEMDKINQQKAELLYGVIDNSDFYRNDVAKANRSRMNVPFQLADSALDKLFLEESFAAGLHALKGHRVVGGMRASIYNAMPLEGVKALTDFMVEFERRHG

SEQ ID NO: 118 - protein, 322 aa, Escherichia coli [sequence omitted]

SEQ ID NO: 119 - protein, 387 aa, Escherichia coli
MSVIKMTDLDLAGKRVFIRADLNVPVKDGKVTSDARIRASLPTIELALKQGAKVMVTSHLGRPTEGEYNEEFSLLPVVNYLKDKLSNPVRLVKDYLDGVDVAEGELVVLENVRFNKGEKKDDETLSKKYAALCDVFVMDAFGTAHRAQASTHGIGKFADVACAGPLLAAELDALGKALKEPARPMVAIVGGSKVSTKLTVLDSLSKIADQLIVGGGIANTFIAAQGHDVGKSLYEADLVDEAKRLLTTCNIPVPSDVRVATEFSETAPATLKSVNDVKADEQILDIGDASAQELAEILKNAKTILWNGPVGVFEFPNFRKGTEIVANAIADSEAFSIAGGGDTLAAIDLFGIADKISYISTGGGAFLEFVEGKVLPAVAMLEERAKK

SEQ ID NO: 120 - DNA, 41 bp, synthetic construct
  source 1..41: mol_type = other DNA
  modified_base 10: mod_base = OTHER; note = Uracil
agtcctaggtataatgctagctcttaatcatgctaaggagg

SEQ ID NO: 121 - DNA, 29 bp, synthetic construct
  source 1..29: mol_type = other DNA
  modified_base 10: mod_base = OTHER; note = Uracil
attgaagatttgggccatgtggaagtctc

SEQ ID NO: 122 - DNA, 33 bp, synthetic construct
  source 1..33: mol_type = other DNA
  modified_base 10: mod_base = OTHER; note = Uracil
acctaggactgagctagctgtcaaaggcctatg

SEQ ID NO: 123 - DNA, 32 bp, synthetic construct
  source 1..32: mol_type = other DNA
  modified_base 10: mod_base = OTHER; note = Uracil
aatcttcaattttagttctggtccggcaatgc

SEQ ID NO: 124 - DNA, 41 bp, synthetic construct; identical to SEQ ID NO: 120
  source 1..41: mol_type = other DNA
  modified_base 10: mod_base = OTHER; note = Uracil

SEQ ID NO: 125 - DNA, 38 bp, synthetic construct
  source 1..38: mol_type = other DNA
  modified_base 9: mod_base = OTHER; note = Uracil
aaccaagctttattcagctaatccaatccaaggcttgg

SEQ ID NO: 126 - DNA, 33 bp, synthetic construct; identical to SEQ ID NO: 122
  source 1..33: mol_type = other DNA
  modified_base 10: mod_base = OTHER; note = Uracil

SEQ ID NO: 127 - DNA, 29 bp, synthetic construct
  source 1..29: mol_type = other DNA
  modified_base 9: mod_base = OTHER; note = Uracil
agcttggttgataaaagaaggagacttcc

SEQ ID NO: 128 - DNA, 25 bp, synthetic construct
  source 1..25: mol_type = other DNA
acagccggaatcatacttggtttgg

SEQ ID NO: 129 - DNA, 25 bp, synthetic construct
  source 1..25: mol_type = other DNA
aaattcgccatgtgattcaagatcg

SEQ ID NO: 130 - DNA, 25 bp, synthetic construct
  source 1..25: mol_type = other DNA
acagccggaatcatacttggtttgg

SEQ ID NO: 131 - DNA, 25 bp, synthetic construct
  source 1..25: mol_type = other DNA
aaattcgccatgtgattcaagatcg

SEQ ID NO: 132 - DNA, 25 bp, synthetic construct
  source 1..25: mol_type = other DNA
  modified_base 9: mod_base = OTHER; note = Uracil
agtcaccatccgtcacgctgacctg

SEQ ID NO: 133 - DNA, 32 bp, synthetic construct
  source 1..32: mol_type = other DNA
  modified_base 9: mod_base = OTHER; note = Uracil
atggtgacttccgccttttcattcacttttgg

SEQ ID NO: 134 - DNA, 32 bp, synthetic construct; identical to SEQ ID NO: 133
  source 1..32: mol_type = other DNA
  modified_base 9: mod_base = OTHER; note = Uracil

SEQ ID NO: 135 - DNA, 32 bp, synthetic construct
  source 1..32: mol_type = other DNA
  modified_base 9: mod_base = OTHER; note = Uracil
agatccagtaatgacctcagaactccatctgg

SEQ ID NO: 136 - DNA, 30 bp, synthetic construct
  source 1..30: mol_type = other DNA
  modified_base 10: mod_base = OTHER; note = Uracil
agaccagtttatggcggtaacgcaaacagc

SEQ ID NO: 137 - DNA, 35 bp, synthetic construct
  source 1..35: mol_type = other DNA
  modified_base 9: mod_base = OTHER; note = Uracil
acctcagcttactcaaactcattccaggaacgacc

SEQ ID NO: 138 - DNA, 18 bp, synthetic construct
  source 1..18: mol_type = other DNA
  modified_base 9: mod_base = OTHER; note = Uracil
agctgaggtcgcctcagc

SEQ ID NO: 139 - DNA, 33 bp, synthetic construct
  source 1..33: mol_type = other DNA
  modified_base 10: mod_base = OTHER; note = Uracil
aaactggtctccttcttttatcacttctgattc

SEQ ID NO: 140 - DNA, 37 bp, synthetic construct
  source 1..37: mol_type = other DNA
  modified_base 10: mod_base = OTHER; note = Uracil
agaccagtttatgaagcaaacagtttatatcgccagc

SEQ ID NO: 141 - DNA, 31 bp, synthetic construct
  source 1..31: mol_type = other DNA
  modified_base 9: mod_base = OTHER; note = Uracil
acctcagcttagtgtgcgttaaccaccaccc

SEQ ID NO: 142 - DNA, 18 bp, synthetic construct
  source 1..18: mol_type = other DNA
  modified_base 9: mod_base = OTHER; note = Uracil
agctgaggtcgcctcagc

SEQ ID NO: 143 - DNA, 33 bp, synthetic construct; identical to SEQ ID NO: 139
  source 1..33: mol_type = other DNA
  modified_base 10: mod_base = OTHER; note = Uracil

SEQ ID NO: 144 - DNA, 1011 bp, synthetic construct
  source 1..1011: mol_type = other DNA
atggcgttcgcaaaccttcgaaagattttaataagcgattcactggatccatgctgtcgaaagatactgcaagatggcgggttacaggtagtagagaagcaaaacctgtcgaaagaggagttaattgccgagttgcaagactgcgagggtctcattgtgcgttccgcaacaaaggtgaccgcagacgtcatcaatgcggcggagaagttacaagttgtcggacgcgccggaaccggcgtggataatgtggacctcgaggcggcaactagaaagggcgttctggtaatgaacacacccaatggaaactccctgtcagcagcggagctgacctgcggcatgttgatgtgccttgcgcgccaaataccacaagccacggcatcaatgaaggacggcaagtgggaccgcaagaagtttatggggacagaactgaatggtaagacacttggtattctgggtttaggtcggataggccgcgaagtcgcggcacggatgcaggctttcgggatgaaaacagtgggttatgacccgataatcagccccgaggtcgcagcctcatttggggtccaacaattgccgttggaagaaatttggcccctgtgtgacttcataaccgtgcatacgccgctgctgccttctaccacaggtctcctcaatgactctacttttgcccaatgtaagaagggtgtccgggtggtcaactgtgcacgcggcggaatagttgacgagggtgcgctgctgcgggcgcttcaaagtggccagtgtgctggtgcggccttggatgtattcacagaagaacccccgcgcgaccgcgcacttgtggatcacgagaacgtaattagctgccctcacttaggagcatctacaaaggaagcccaatcgcgttgtggggaagaaattgccgtccaattcgttgacatggtgaagggtaaatcattaacgggagtagttaatgcacaggcccttacgagtgccttctctccacataccaagccttggattggattagctgaa